(19) United States
(12) Patent Application Publication
(10) Pub. No.: US 2024/0228989 A1
(43) Pub. Date: Jul. 11, 2024

(54) REVERSE TRANSCRIPTASE VARIANTS FOR IMPROVED PERFORMANCE

(71) Applicant: 10X GENOMICS, INC., Pleasanton, CA (US)

(72) Inventors: Derek Hunter Vallejo, Pleasanton, CA (US); Yufeng Qian, Union City, CA (US); Javelin C. Chi, San Francisco, CA (US)

(73) Assignee: 10X GENOMICS, INC., Pleasanton, CA (US)

(21) Appl. No.: 18/538,539

(22) Filed: Dec. 13, 2023

Related U.S. Application Data
(63) Continuation of application No. PCT/US22/33199, filed on Jun. 13, 2022.
(60) Provisional application No. 63/210,143, filed on Jun. 14, 2021.
(60) Provisional application No. 63/290,329, filed on Dec. 16, 2021.

(51) Int. Cl. C12N 9/12; C12N 15/10

(52) U.S. Cl. CPC C12N 9/1276; C12N 15/1096

(57) ABSTRACT

The disclosure provides engineered reverse transcriptase enzymes that have been modified to enhance their enzymatic to increase their processivity, template switching efficiency, binding affinity, and/or transcription efficiency and to decrease their RNAse H activity. The disclosure further provides compositions and kits comprising the engineered reverse transcriptase enzymes and methods of producing, amplifying or sequencing nucleic acid molecules using these reverse transcriptase enzymes.

### CROSS-REFERENCE TO RELATED PATENT APPLICATIONS

This application is a continuation of International Patent Application No. PCT/US2022/033199, filed Jun. 13, 2022, which claims priority to U.S. Provisional Patent Application No. 63/210,143, filed on Jun. 14, 2021, and U.S. Provisional Patent Application No. 63/290,329, filed on Dec. 16, 2021, the contents of which are incorporated herein by reference in their entireties.

### SEQUENCE LISTING

The instant application contains a Sequence Listing which has been submitted electronically in XML format and is hereby incorporated by reference in its entirety. Said XML file was created on Mar. 20, 2024, is named 131488_0204_Sequence_Listing.xml, and is 78 bytes in size.

### FIELD OF INVENTION

The present invention relates to the field of protein engineering and enzymatics, particularly the development of reverse transcriptase variants.

### BACKGROUND

The discovery of reverse transcriptase (RT) in the 1970's revolutionized the understanding of eukaryotic biology by demonstrating that genetic information did not flow unidirectionally from DNA to RNA to proteins. Rather, the genetic information could also flow in the reverse direction from RNA back to DNA. The ability to convert mature mRNA back into cDNA, without the introns present in genomic DNA is critical for obtaining information in a wide variety of biomedical contexts, including diagnostics, prognostics, biotechnology, and forensic biology. Since then, RT enzymes (RTs) have become ubiquitous tools in molecular biology driving enabling technologies such as next-generation RNA-Sequencing, Maxam-Gilbert sequencing and chain-termination methods, or de novo sequencing methods including shotgun sequencing and bridge PCR, or next-generation methods including polony sequencing, 454 pyrosequencing, Illumina sequencing, SOLID sequencing, Ion Torrent semiconductor sequencing, HeliScope single molecule sequencing, SMRT® sequencing.

RT enzymes were initially found in retroviruses such as Moloney murine leukemia virus (MMLV)). It is now clear that RTs are present in other microorganisms, including transposable elements, where RTs are responsible for converting an RNA genome of these organisms into DNA to facilitate the integration of the microorganisms into a host's chromosome. All known natural RTs are derived from a shared common ancestor. Generally, RTs are mesophilic enzymes that function best at moderate temperatures ranging from 20° ° C. to 45° C. The mesophilic nature of RTs is problematic for in vitro amplification reactions because RNAs tend to adopt stable secondary structures at lower temperatures resulting in inefficient reverse transcription reactions at these low to moderate temperatures. In addition to the RNA secondary structures, RT reactions and amplification reactions also fail because biological samples from which nucleic acids are extracted often contain additional compounds that are inhibitory to reverse transcription and/or amplification reactions. This inhibition is particularly problematic when the volume of an amplification reaction is very small (e.g., nanoliter), such as in single cell profiling reactions and additional methods where small reaction volumes are preferential.

Accordingly, there is a need for improved reverse transcriptases with improved properties, such as improved efficiency, processivity, thermoreactivity, and/or thermostability. The present disclosure addresses this need.

### SUMMARY OF THE INVENTION

One aspect of the present disclosure provides an engineered reverse transcriptase comprising the amino acid sequence of SEQ ID NO: 15, and further comprising a combination of mutations selected from: (a) E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or (b) E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P.

In some embodiments, the engineered reverse transcriptase comprises an amino acid sequence that is at least 90% identical to an amino acid sequence selected from the group consisting of: SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO: 13, SEQ ID NO: 14, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:30, SEQ ID NO:31, SEQ ID NO:32, SEQ ID NO:33, SEQ ID NO:34, SEQ ID NO:35, SEQ ID NO:36 and SEQ ID NO:37.

In some embodiments, the engineered reverse transcriptase exhibits an enhanced reverse transcriptase activity as compared to a reverse transcriptase having the amino acid sequence set forth in SEQ ID NO:1. In some embodiments, the enhanced reverse transcriptase activity is selected from the group consisting of processivity, template switching efficiency, binding affinity, and transcription efficiency.

In some embodiments, the enhanced reverse transcriptase activity is an enhanced template switching (TS) efficiency as compared to the template switching efficiency of a reverse transcriptase having the amino acid sequence set forth in SEQ ID NO:1.

In some embodiments, the enhanced reverse transcriptase activity is an enhanced transcription efficiency as compared to the transcription efficiency of a reverse transcriptase having the amino acid sequence set forth in SEQ ID NO:1.

In some embodiments, the enhanced reverse transcriptase activity is the enhanced transcription efficiency and template switching efficiency as compared to a reverse transcriptase having the amino acid sequence set forth in SEQ ID NO:1.

In some embodiments, the enhanced reverse transcriptase activity is the increased binding affinity as compared to the binding affinity of a reverse transcriptase having the amino acid sequence set forth in SEQ ID NO:1.

In some embodiments, the enhanced reverse transcriptase activity is an increased binding affinity and template switching efficiency as compared to a reverse transcriptase having the amino acid sequence set forth in SEQ ID NO:1.

In some embodiments, the enhanced reverse transcriptase activity is an enhanced processivity as compared to the processivity of a reverse transcriptase having the amino acid sequence set forth in SEQ ID NO:1.

In some embodiments, the enhanced reverse transcriptase activity is an enhanced ability to yield mitochondrial UMI counts as compared to a reverse transcriptase having the amino acid sequence set forth in SEQ ID NO:1.

In some embodiments, the enhanced reverse transcriptase activity is an enhanced ability to yield ribosomal UMI counts as compared to a reverse transcriptase having the amino acid sequence set forth in SEQ ID NO:1.

In some embodiments of the engineered reverse transcriptase described herein, the amino acid sequence of the engineered reverse transcriptase comprises E69K, L139P, D200N, E302R, T306K, W313F, T330P, N454K, H503V, D524N, L603W, E607K, and H634Y. In that embodiments, the amino acid sequence of the engineered reverse transcriptase further comprises a combination of mutations selected from the group consisting of: (a) M66L and L435G; (b) M39V, M66L, and L435K; (c) M39V and L435K; (d) M66L, L435G, P448A and D449G; (e) M39V, M66L, L435G, P448A and D449G; and (f) M66L.

In some embodiments of the engineered reverse transcriptase described herein, the amino acid sequence of the engineered reverse transcriptase comprises E69K, L139P, D200N, E302R, T306K, W313F, T330P, L435G, P448A, D449G, N454K, D524N, L603W, and E607K; and further comprises a combination of mutations selected from the group consisting of (a) M66L; (b) M66L and H503V; (c) M66L and H634Y; and (d) M66L, H503V, and H634Y.

In some embodiments of the engineered reverse transcriptase described herein, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P and further comprising a second combination of mutations selected from the group consisting of: (a) D524N, T542D, P627S, A644V, D653H, K658R mutation, and wherein said D200 mutation is a D200N mutation, said D449 mutation is a D449G, said L603 mutation is an L603W, and said E607 mutation is an E607G mutation; (b) D524N, T542D, A644V, D653H, an R650H and K658R, and wherein said D200 mutation is a D200N mutation, said D449 mutation is a D449E mutation, said L603 mutation is an L603W mutation, and said E607 mutation is an E607G mutation; (c) E545G, D583N, and H594Q, and wherein said D200 mutation is a D200N mutation, said D449 mutation is a D449G mutation, said L603 mutation is an L603F mutation, and said E607 mutation is an E607K mutation; (d) D524N, T542D, A644V, D653H, and K658R, wherein said D200 mutation is a D200N mutation, said D449 mutation is a D449E mutation, said L603 mutation is an L603W mutation, said E607 mutation is an E607G mutation; (e) H204R, D524N, T542D, P627S, D583N, A644V, D653H and K658R, wherein said D200 mutation is a D200E mutation, said D449 mutation is a D449G mutation, said L603 mutation is an L603W mutation, said E607 mutation is an E607G mutation; (f) H204R, E545G, D583N, and H594Q, wherein said D200 mutation is a D200E mutation, said D449 mutation is a D449G mutation, said L603 mutation is an L603F mutation, said E607 mutation is an E607K mutation; and (g) P47L, D524N, T542D, D583N, P627S, A644V, D653H, and K658R, wherein said D200 mutation is a D200N mutation, said D449 mutation is a D449G mutation, said L603 mutation is an L603W mutation, said E607 mutation is an E607G mutation.

Another aspect of the present disclosure provides an engineered reverse transcriptase comprising an amino acid sequence selected from the group consisting of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, SEQ ID NO: 13, SEQ ID NO:14, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:30, SEQ ID NO:31, SEQ ID NO:32, SEQ ID NO:33, SEQ ID NO:34, SEQ ID NO:35, SEQ ID NO:36 and SEQ ID NO:37.

In some embodiments, the engineered reverse transcriptase exhibits an enhanced reverse transcriptase activity as compared to a reverse transcriptase having the amino acid sequence set forth in SEQ ID NO:1.

In some embodiments, the enhanced reverse transcriptase activity is selected from the group of reverse transcriptase related activities comprising processivity, template switching efficiency, binding affinity and transcription efficiency.

Another aspect of the present disclosure provides an engineered reverse transcriptase comprising the amino acid sequence of SEQ ID NO:15, and further comprising a combination of mutations selected from the group consisting of T542D, D583N, E607G, A644V, D653H, K658R, E545G, D583N, H594Q, and a L603F.

In some embodiments, the engineered transcriptase comprises: (a) an amino acid sequence that is at least about 90%, at least about 92%, at least about 95%, at least about 97%, at least about 98%, or at least about 99% identical to an amino acid sequence selected from the group consisting of SEQ ID NO: 8, SEQ ID NO: 9, SEQ ID NO: 10, SEQ ID NO: 11, SEQ ID NO: 12, SEQ ID NO: 13, and SEQ ID NO: 14; or (b) an amino acid sequence selected from the group consisting of SEQ ID NO: 8, SEQ ID NO: 9, SEQ ID NO: 10, SEQ ID NO: 11, SEQ ID NO: 12, SEQ ID NO: 13, and SEQ ID NO: 14.

In some embodiments, the engineered reverse transcriptase exhibits an enhanced reverse transcriptase activity as compared to a reverse transcriptase having the amino acid sequence set forth in SEQ ID NO:1.

In some embodiments, the enhanced reverse transcriptase activity is selected from the group of reverse transcriptase related activities comprising an RNAase H activity, processivity, template switching efficiency, binding affinity and transcription efficiency.

Another aspect of the present disclosure provides an engineered reverse transcriptase comprising an amino acid sequence that is at least 90% identical to the amino acid sequence of SEQ ID NO: 2.

Another aspect of the present disclosure provides an engineered reverse transcriptase comprising an amino acid sequence that is at least 90% identical to the amino acid sequence of SEQ ID NO: 24.

In some embodiments, the engineered reverse transcriptase possesses one or more of the following characteristics when compared to a wild-type reverse transcriptase or a reverse transcriptase comprising the amino acid of SEQ ID NO: 1: (a) increased thermostability; (b) increased thermoreactivity; (c) increased resistance to reverse transcriptase inhibitors; (d) increased ability to reverse transcribe difficult templates; (e) increased speed; (f) increased processivity; (g) increased specificity; (h) enhanced polymerization activity; or (i) increased sensitivity.

In that embodiment: (a) the increase in thermoreactivity, resistance to reverse transcriptase inhibitors, ability to reverse transcribe difficult templates, speed, processivity, specificity, or sensitivity is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type reverse transcriptase or a reverse transcriptase comprising the amino acid of SEQ ID NO: 1; or (b) the polymerization activity is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type reverse transcriptase or a reverse transcriptase comprising the amino acid of SEQ ID NO: 1.

One aspect of the present disclosure provides an isolated nucleic acid molecule encoding the engineered reverse transcriptase described herein.

Another aspect of the present disclosure provides an expression vector comprising the isolated nucleic acid described herein.

Another aspect of the present disclosure provides a host cell transfected with the expression vector described herein.

One aspect of the present disclosure provides a method of using the engineered reverse transcriptase described herein, the method comprising contacting the engineered reverse transcriptase with a nucleic acid template under suitable conditions to produce a polymerized nucleic acid product, wherein the nucleic acid template is an RNA, a DNA, or a nucleic acid comprising an unnatural nucleotide.

One aspect of the present disclosure provides a nucleic acid extension method comprising: (a) contacting a target nucleic acid molecule with an engineered reverse transcriptase and a plurality of nucleic acid barcoded molecules comprising a barcode sequence, and (b) incubating the target nucleic acid, the engineered reverse transcriptase and barcoded molecules under conditions in which the barcoded molecules are extended by the engineered reverse transcriptase, wherein the engineered reverse transcriptase comprises the amino acid sequence of an engineered transcriptase described herein.

### BRIEF DESCRIPTION OF THE DRAWINGS

FIGS. 1A-G show a CLUSTAL O (1.2.4) multiple protein alignment reports of the wild-type (WT) and engineered Moloney Murine Leukemia Virus reverse-transcriptase (MMLV RT) variants disclosed herein. FIG. 1A shows an alignment illustrating the difference between an engineered MMLV RT variant (SEQ ID NO:1) and the wt MMLV(SEQ ID NO: 15; GenBank Seq ID NP_955591.1 p80RT(ebi.ac.uk/Tools/msa/clustalo/)). The MMLV RT variant of SEQ ID NO: 1 is the RT enzyme found in enzyme mix C (EMC) and was used as a control in the Examples disclosed herein. FIGS. 1B-H show the similarities and differences between WT MMLV RT, MMLV RT variant of SEQ ID NO: 1, and the novel MMLV RT variants disclosed herein (Table 2).

FIG. 2 shows an exemplary schematic of a capillary electrophoresis (CE) validation assay process disclosed herein. Specifically, 5′-end labeled DNA primers are initially hybridized to RNA templates at room temperature (approx. 25° C.); then poly rG-labeled template switching oligonucleotides (rG-TSO) are added to the reaction mixture. The temperature is raised to 53° C. to initiate the first strand cDNA synthesis and the addition of a poly-C tail. The hybridization of the rG-TSO oligonucleotide and TSO extension occur. Finally, extended samples are transferred to a SeqStudio™ Genetic Analyzer for analysis.

FIG. 3 shows an exemplary trace of a CE assay output. The product size was calibrated with synthetically sized controls for the primer alone size, a full-length extension of the primer length, and a full-length extension of the primer plus template switching oligo. Product length is indicated on the x-axis and signal intensity is indicated on the y-axis.

FIGS. 4A-B show exemplary traces of a CE assay output for enzyme controls for enzyme mix C (FIG. 4B) which contains an engineered reverse transcriptase and a transcription positive, template switching null engineered reverse transcriptase (listed as AR; FIG. 4A). Product length is indicated on the x-axis; signal intensity is indicated on the y-axis. Peaks associated with the full-length product, the full-length product plus tail, and the full length product plus tail and TSO are indicated.

FIG. 5 show an exemplary trace of a CE assay output for an enzyme mix C as described in FIG. 1, including length parameters that are associated with various reaction products. The length parameters are used for transcription efficiency and template switching efficiency calculations.

FIGS. 6A-B show bar graph summarizing results obtained from CE analysis of various reverse transcriptase variants compared to the variant MMLV RT of SEQ ID NO:1. The variant is indicated on the x-axis of each chart. The y-axis indicates the fraction of full-length product (FIG. 6A) and the fraction of template switched product (FIG. 6B) when the listed RT variant is utilized in a reverse transcription and template switching oligonucleotide assay, respectively.

FIG. 7 shows an exemplary bar graph comparing the transcription efficiency and template switching efficiency (TSO efficiency) of multiple engineered reverse transcriptases disclosed herein in CE assays using a GAPDH RNA (SEQ ID NO: 18) sequence as a template for reverse transcription. Bars indicating the transcription efficiency are indicated on the left (dark grey) for each enzyme tested; bars indicating the template switching efficiency are indicated on the right (light grey) for each enzyme tested. The percent product is indicated on the y axis; the variant enzymes tested are indicated on the x axis.

FIG. 8 shows an exemplary table comparing the transcription efficiency, template switching efficiency and fraction of product (plus TSO) of multiple engineered reverse transcriptase variants (SEQ ID NOs: 22, 23, 21, 4, 3, 5, 24, 2, and 7) compared to control SEQ ID NO:1 in CE assays performed using a GAPDH RNA template (SEQ ID NO: 18). Variants include different mutational site combinations (wt MMLV position of SEQ ID NO: 15), as listed under ‘MMLV Position’.

FIG. 9 shows an exemplary bar graph summarizing the cDNA yields obtained from a control engineered reverse transcriptase (MMLV RT; SEQ ID NO: 1) compared to variants MMLV RT disclosed herein (SEQ ID NOs: 22, 24, 2, 3 and 7) in single cell experiments. The single cell experiments were performed in either a 3′ (sc-3′ left) or 5′ (sc-5′ right) experimental design.

FIGS. 10A-C show exemplary tables summarizing metrics of single cell gene expression experiments generated with a control RT (SEQ ID NO: 1; a known MMLV RT variant) and engineered MMLV RT variants disclosed herein (SEQ ID NOs: 22, 24, 2, 3 and 7); FIG. 10A shows results from 20 k read metrics for median genes and median UMIs per cell, FIG. 10B shows results from 50K read metrics for median genes and median UMIs per cell, and FIG. 10C shows read results that were mapped to the transcriptome in single cells. The percent indicates the percent change from the control SEQ ID NO:1.

FIGS. 11A-B show exemplary tables summarizing metrics related to results obtained from engineered MMLV RT variants disclosed herein in 3′ single cell experiments from FIGS. 10A-C.

FIG. 12 shows an exemplary table summarizing metrics of 5′ single cell experiments, including 20 k read metrics, 50K read metrics and reads mapped to the transcriptome, using the same control and engineered MMLV RT variants of FIGS. 10A-C. The percent indicates the percent change from control SEQ ID NO:1.

FIGS. 13A-B show exemplary tables summarizing metrics related to single cell 5′ experiments of FIG. 12.

FIGS. 14A-B show exemplary tables reporting gene expression (GEX) metrics in different single cell types using control engineered MMLV RT (SEQ ID NO: 1) compared to engineered MMLV RT variants disclosed herein (SEQ ID NOs: 2, 25, 24, or 7).

FIGS. 15A-C show exemplary scatter plots (FIGS. 15A-B) and a t-distributed stochastic neighbor embedding (t-SNE) plot (FIG. 15C) of single cell gene expression results using 5′ single cell chemistry in human PBMCs and in mouse PBMCs (C57BL/6 cells) comparing two engineered MMLV RT variants disclosed herein (SEQ ID NOs: 2 and 7).

FIG. 16 shows an exemplary table summarizing immune profiling results from experiments comparing a control Enzyme mix C (control engineered MMLV RT; SEQ ID NO: 1) with three engineered MMLV RT variants disclosed herein (SEQ ID NOs: 2, 25 and 24). Percent change is relative to the control.

FIG. 17 shows a schematic diagram of a generalized capture probe used in spatial transcriptomics and single cell transcriptomic analyses, exemplary applications in addition to general reverse transcription reactions where the engineered thermostable reverse transcriptase of the invention could be used to extend a capture probe using a captured target nucleic acid as a template, thereby generating a cDNA product.

### DETAILED DESCRIPTION

It is to be appreciated that certain aspects, modes, embodiments, variations and features of the present methods are described below in various levels of detail in order to provide a substantial understanding of the present technology.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Overview

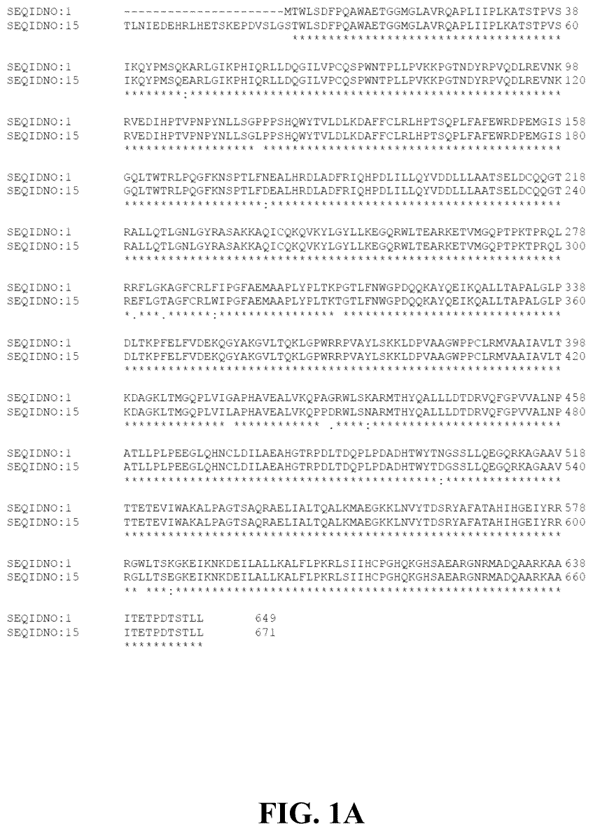

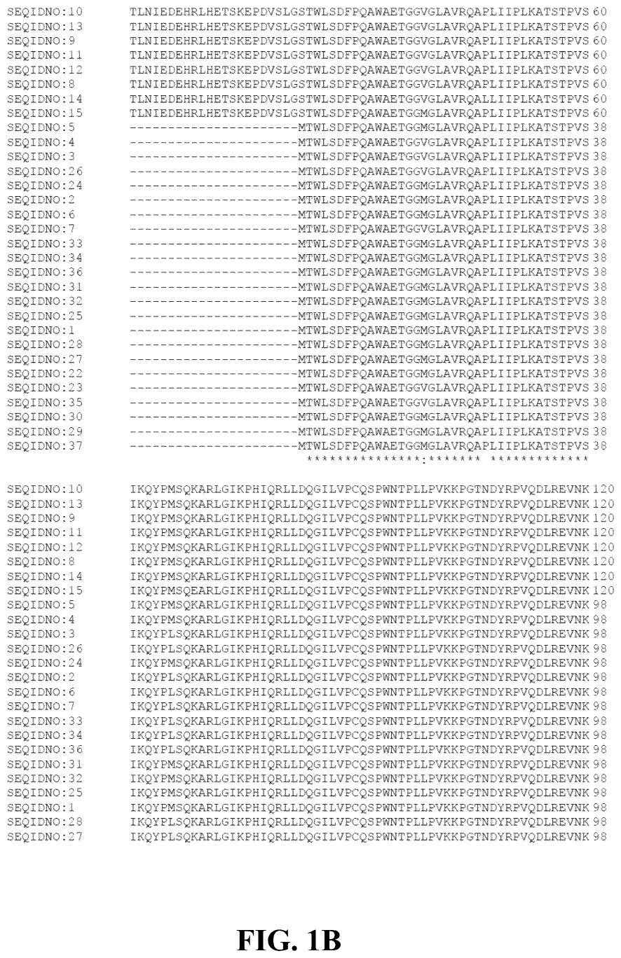

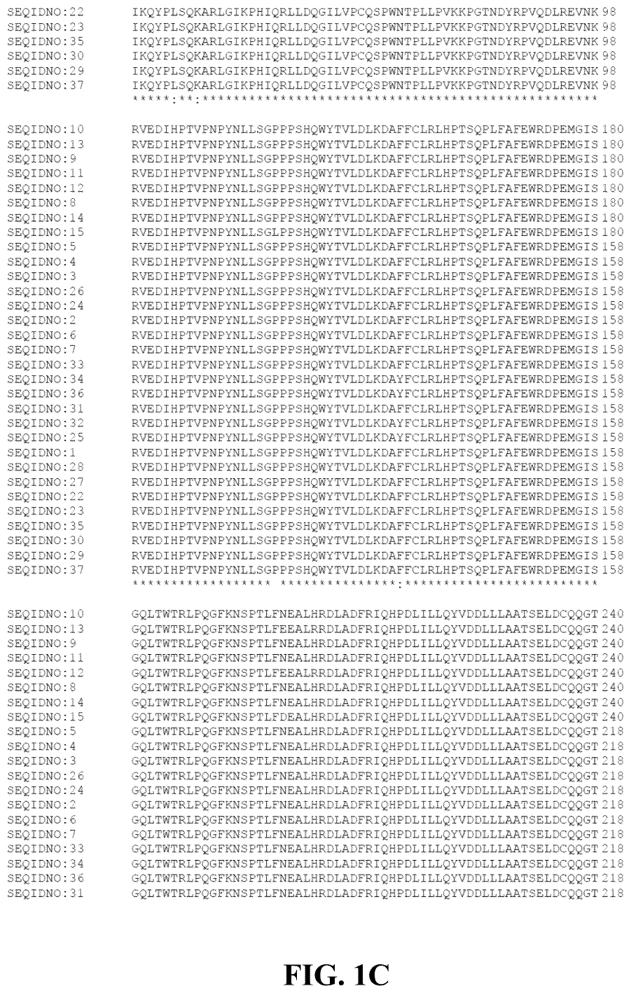

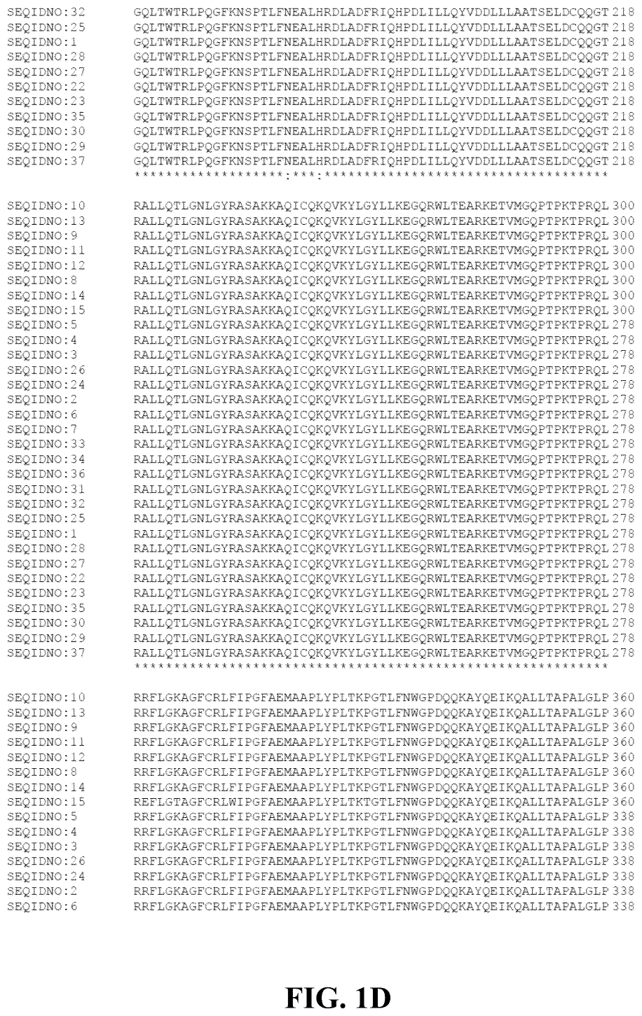

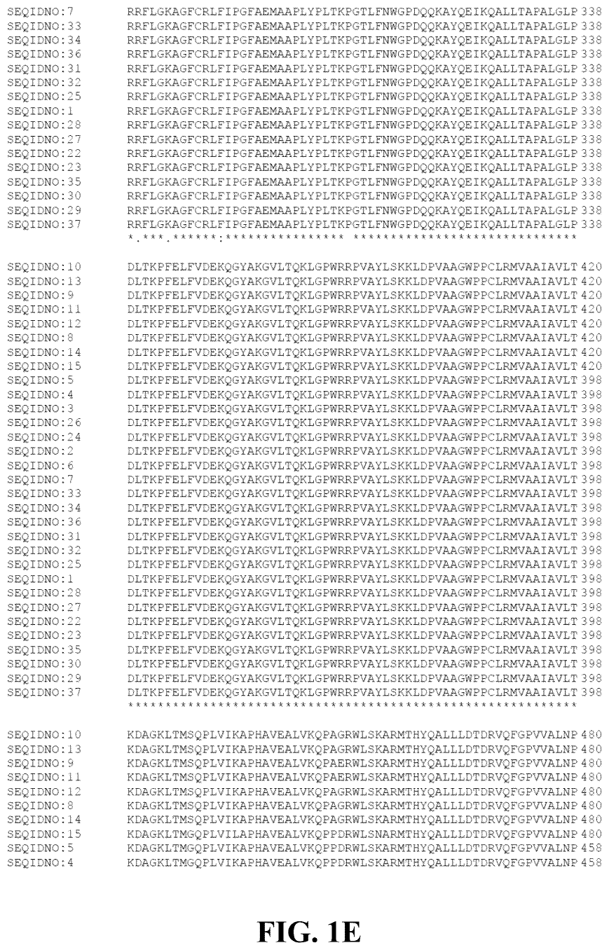

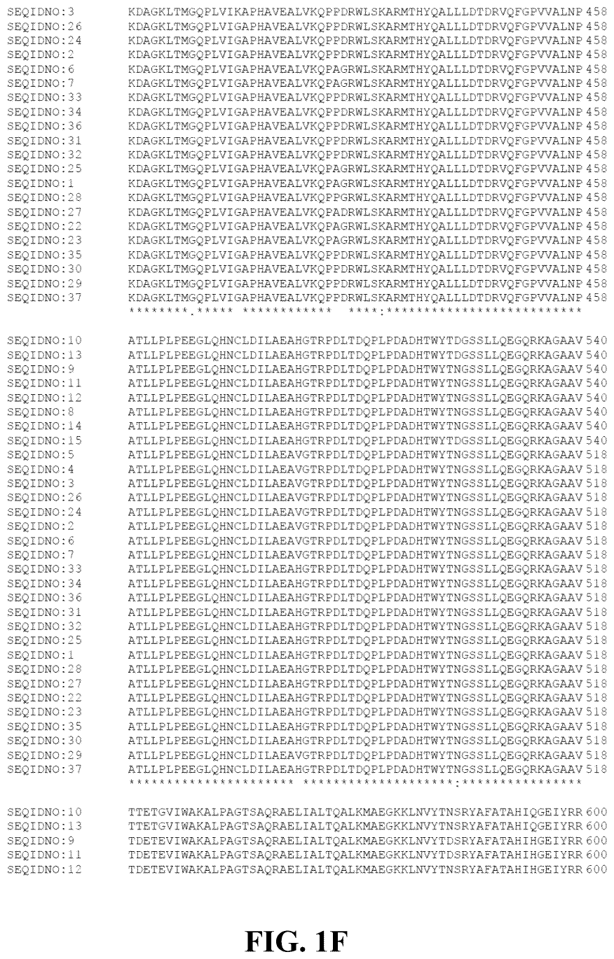

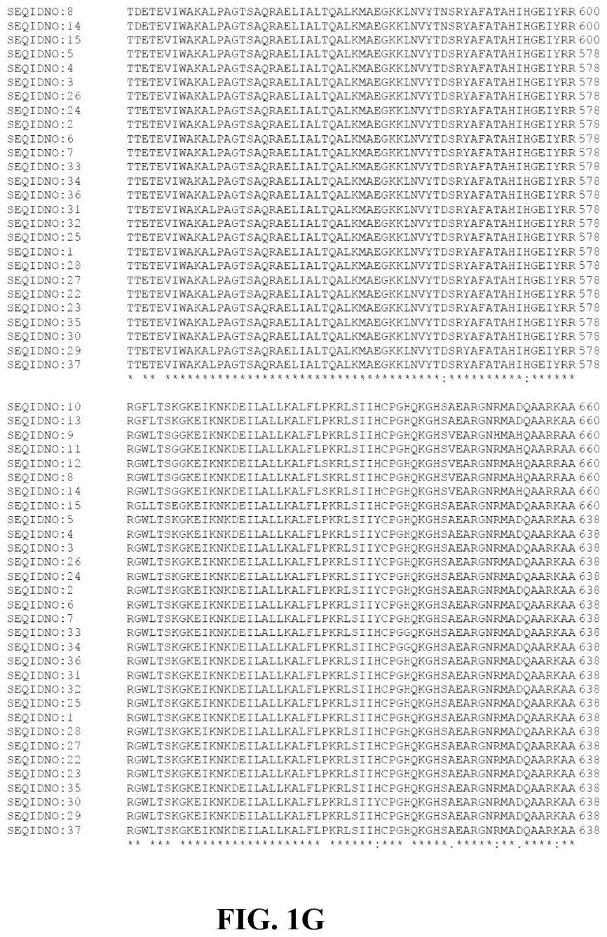

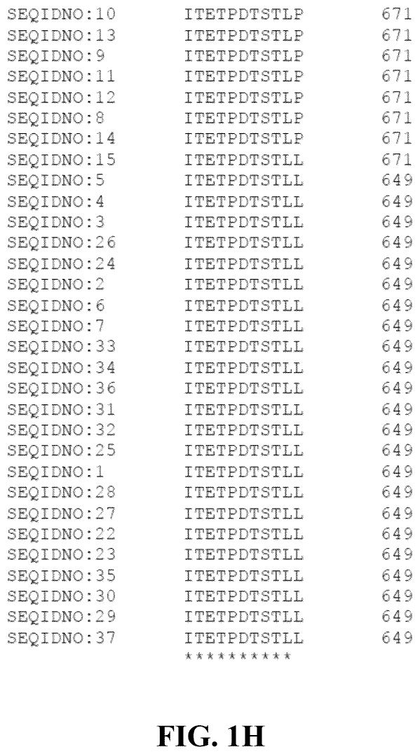

A challenge in cDNA synthesis reactions is interference from RNA secondary structures. While a higher reaction temperature can remove secondary structure from the template RNA, elevated temperatures typically lead to lower reverse-transcriptase (RT) enzyme activity if the enzyme is not nascently thermostable. Additionally, RT enzyme activity can be reduced by inhibitors, such as those which might be found in cell lysates and associated reagents. Wild-type (WT) Moloney Murine Leukemia Virus (MMLV) reverse-transcriptase is an RT enzyme that is typically inactivated at higher temperatures. Several commercially available mutant MMLV RT enzymes have been generated that exhibit improved thermostability, fidelity, substrate affinity, and/or reduced terminal deoxynucleotidyltransferase activity. For example, specific residues of MMLV, such as M39V, M66L, E69K, E302R, T306K, W313F, L/K435G, and N454K of the wild-type MMLV (SEQ ID NO: 15) have been shown to improve thermostability of the wild-type RT MMLV. See e.g., Arezi et al Nucleic Acids Res. 37(2):473-481 (2009), U.S. Pat. No. 7,078,208, and Baranauskas et al., Prot. Eng. 25(10): 657-668 (2012); and FIG. 1A.

While these variants MMLV RT may function well in routine amplification reactions, these variants are not optimal for reverse transcription of mRNA when using high throughput amplification reaction assays (e.g. spatial arrays and single cell transcriptomics assays) and the like. This is because high throughput amplification reaction assays require reaction volumes that are usually less than about 1 nanoliter. In addition, sample processing chemicals can negatively impact the function and activity of wild-type and available MMLV variant.

Accordingly, the present disclosure provides an engineered reverse transcriptase comprising the amino acid sequence of SEQ ID NO: 15, and further comprising a combination of mutations selected from: E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or further comprising a combination of mutations selected from: E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P. In some embodiments, the engineered reverse transcriptase exhibits an enhanced reverse transcriptase activity as compared to a reverse transcriptase having the amino acid sequence set forth in SEQ ID NO:1 or 15.

In another aspect, the present disclosure provides an engineered reverse transcriptase, comprising an amino acid sequence selected from the group consisting of SEQ ID NO:2, SEQ ID NO:3, SEQ ID NO: 4, SEQ ID NO:5, SEQ ID NO:6, SEQ ID NO: 7, SEQ ID NO:8, SEQ ID NO:9, SEQ ID NO:10, SEQ ID NO:11, SEQ ID NO: 12, SEQ ID NO:13, SEQ ID NO:14, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:30, SEQ ID NO:31, SEQ ID NO:32, SEQ ID NO:33, SEQ ID NO:34, SEQ ID NO:35, SEQ ID NO:36, and SEQ ID NO:37.

In another aspect, the present disclosure provides an engineered reverse transcriptase comprising the amino acid sequence of SEQ ID NO: 15, and further comprising a combination of mutations selected from the group consisting of T542D, D583N, E607G, A644V, D653H, K658R, E545G, D583N, H594Q, and a L603F. In another aspect, the present disclosure provides an engineered reverse transcriptase comprising an amino acid sequence that is at least 90% identical to the amino acid sequence of SEQ ID NO: 2 or 24.

The present disclosure also provides isolated nucleic acid molecules encoding the novel engineered reverse transcriptase disclosed herein, expression vectors comprising the isolated nucleic acid molecules, and host cells transfected with the isolated nucleic acid molecules or expression vectors comprising the novel engineered reverse transcriptase disclosed herein. In another aspect, the present disclosure provides a method of using the engineered reverse transcriptase disclosed herein or a nucleic acid extension method comprising the engineered reverse transcriptase.

### A. Summary of Experimental Results

As noted above, reverse transcription of mRNA from a single cell can be inhibited when the reaction volume is less than about 1 nL. In addition, sample processing chemicals can negatively impact the function and activity of wild-type and available MMLV variants. Overcoming the inhibitory effects of the reaction volume and processing chemical impact has been a challenge for efficiently performing single cell profiling using mRNA. As shown in the examples below, the novel MMLV variants disclosed herein overcome these challenges.

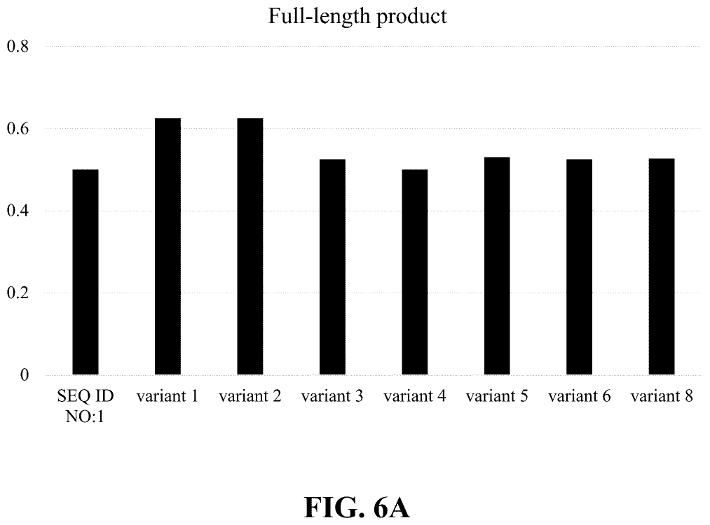

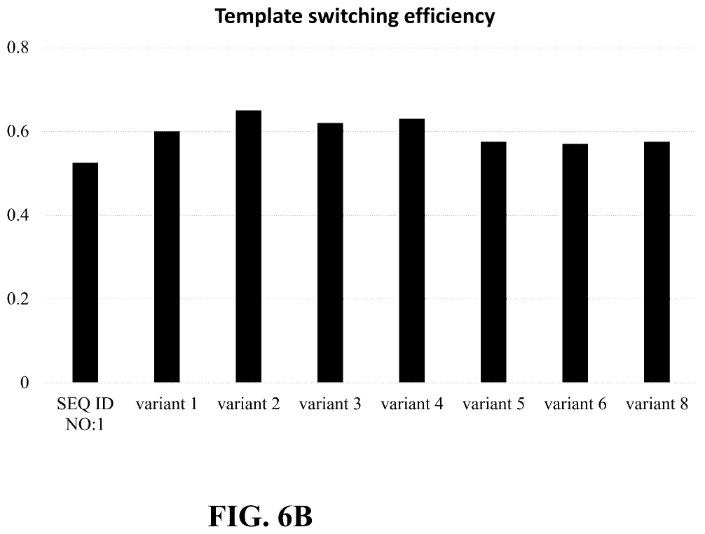

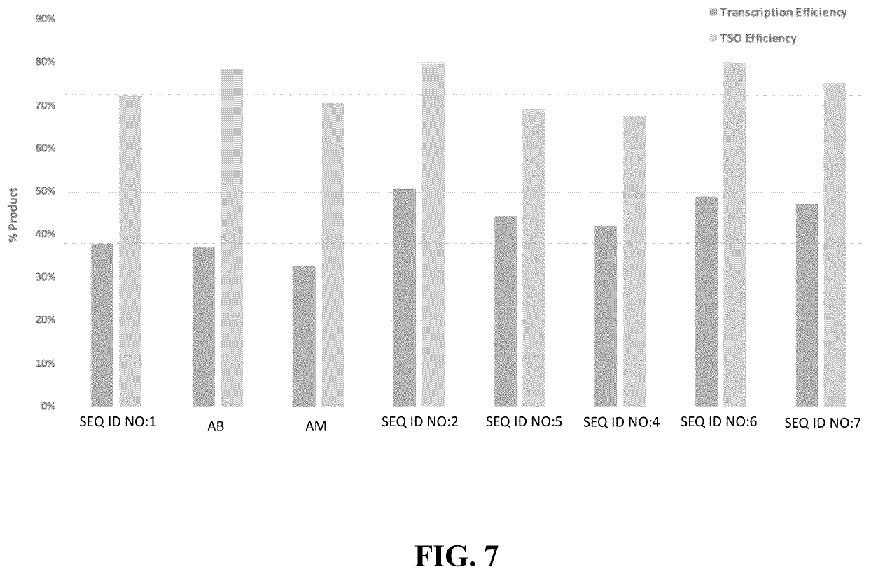

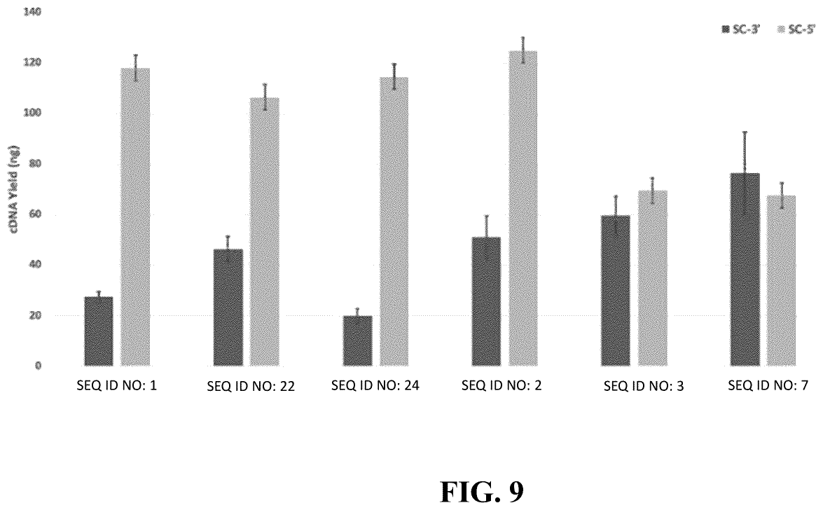

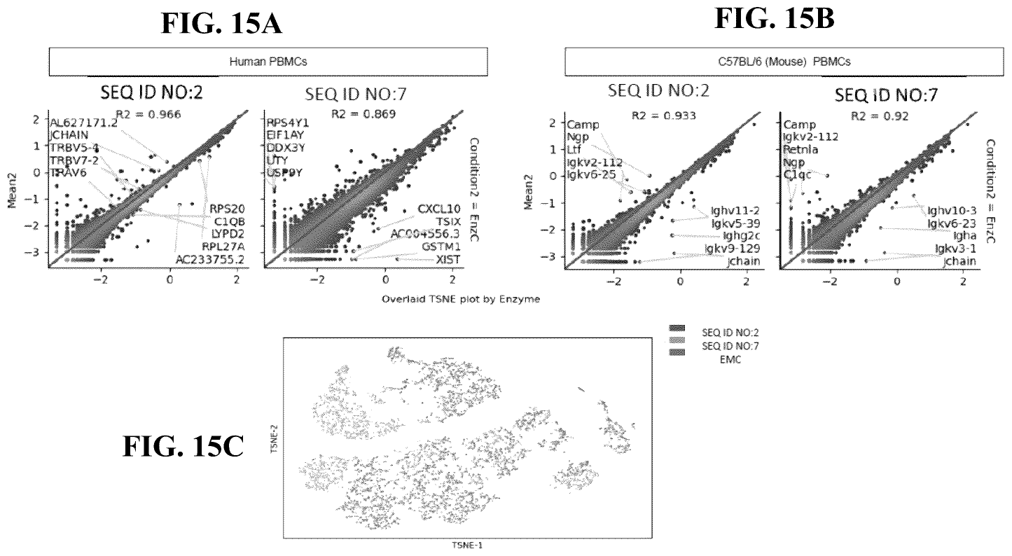

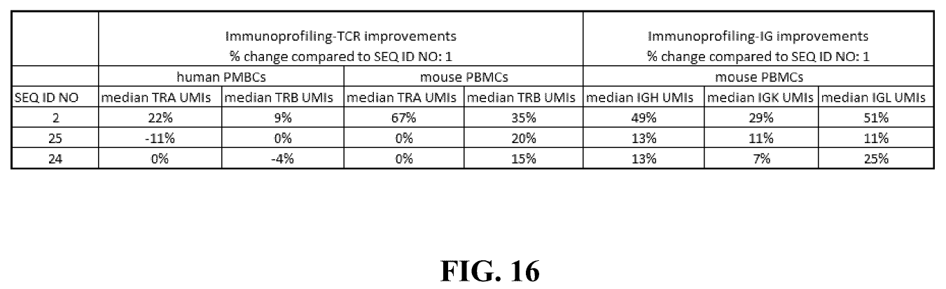

The novel class of MMLV variants described herein (FIGS. 1B-1H) exhibit a combination of reverse transcriptase activity and high thermostability in routine RT-PCR amplification and in high throughput amplification reaction assays, such as single cell profiling using mRNA. As shown in FIGS. 6 and 7, all the MMLV variants disclosed herein showed significant enhancement in the transcription efficiency and template switching in a single cell profiling assay when compared to a wild-type MMLV or a variant MMLV comprising the amino acid sequence of SEQ ID NO: 1. In particular, variants comprising M66L, H503 or H634 mutations either alone or in combination, in a wild-type (SEQ ID NO: 15) or a variant (SEQ ID NO: 1) background, showed superior transcription efficiency and template switching. See FIGS. 6-9. These novel variants also showed enhanced efficiency in all tested parameters of the single cell profiling assays as shown in FIGS. 9-14. Furthermore, the novel variant MMLV RT enzymes disclosed herein showed a dramatic enhancement in gene expression (GEX) sensitivity and mapping (FIGS. 15 and 16) using human and mouse peripheral blood monocytes.

### B. Exemplary Benefits of the Novel MMLV RT Variants

Accordingly, the combination of mutations in each of the variants disclosed herein was unexpectedly sufficient to overcome the inhibitory effects of: (1) low volume high throughput amplification reaction volumes (i.e., less than about 1 nanoliter) which could lead to (2) chemically crowded reaction conditions, and (3) sample processing chemicals, on the function and activity of the wild-type and/or available MMLV variants. Many of these substitutions were surprising and unexpected. For example, the P448A and D449G substitutions in SEQ ID NO: 1 were reverted to wild-type in the majority of the novel MMLV variant disclosed herein as further experimentation demonstrated these two mutations were not as advantageous as originally expected. In addition, residues that were already mutated in SEQ ID NO: 1 were further mutated to generate a novel variant with improved transcriptional activity in the high throughput amplification reaction assays. For example, as shown in FIGS. 1B-1H, D200N was mutated to D200E in some variants; L435G was mutated to L435K in some variants; L603W was mutated to L603F in some variants; and E607K was mutated to E607G in some variants.

Furthermore, the engineered reverse transcriptase variants described herein unexpectedly exhibited higher resistance to cell lysate inhibitory effects than that exhibited by an enzyme having the amino acid sequence set forth in SEQ ID NO: 1 or 15. Lastly, the engineered reverse transcriptase variants of the present disclosure unexpectedly showed greater ability to capture full-length transcripts in T-cell receptor paired transcriptional profiling, as compared to that exhibited by an enzyme having the amino acid sequence set forth in SEQ ID NO: 1 or 15.

The engineered reverse transcriptase variants described herein can be used with any applications that require RNA amplification. A wide variety of different applications of cell processing and analysis methods and systems are known in the art, including analysis of specific individual cells, analysis of different cell types within populations of differing cell types, analysis and characterization of large populations of cells for environmental, human health, epidemiological forensic, or any of a wide variety of different applications.

### II. An Engineered Reverse Transcriptase

Reverse transcriptases or reverse transcription (RT) enzymes are RNA-dependent DNA polymerases, typically used to create a copy of an RNA sequence thereby generating a cDNA molecule. Reverse transcription is initiated by hybridization of a priming sequence to an RNA molecule which is extended by a reverse transcription enzyme in a template directed fashion. A reverse transcription enzyme adds a plurality of non-template nucleotides to a nucleotide strand, thereby producing complementary deoxyribonucleic acid (cDNA) molecules. The resultant cDNA can then be dehybridized from the template RNA molecule in any number of ways as known in the art.

### A. Novel Variants

In one aspect, the present disclosure provides an engineered reverse transcriptase comprising the amino acid sequence of SEQ ID NO:15, and further comprising a combination of mutations selected from the group consisting of: E69K, L139P, E302R, T306K, W313F, T330P, N454K; and one or more of M39V, P47L, M66L, F155Y, D200N, D200E, H204R, G429S, L435G, L435K, P448A, D449G, H503V, D524N, T542D, E545G, D583N, H594Q, L603W, L603F, E607K, E607G, P627S, H634Y, H638G, A644V, D653H, K658R and L671P; or further comprising a combination of mutations selected from the group consisting of E69K, L139P, D200N, E302R, T306K, W313F, T330P, L435G, P448A, D449G, N454K, D524N, L603W, and E607K and one or more of M39V, P47L, M66L, F155Y, H204R, G429S, H503V, T542D, E545G, D583N, H594Q, P627S, H634Y, H638G, A644V, D653H, K658R and L671P.

The engineered reverse transcriptase of the present disclosure is a variant Moloney Murine Leukemia Virus (MMLV) reverse-transcriptase having one or more mutations. Specifically, the novel engineered reverse transcriptase described herein comprises a combination of mutations in the amino acid sequence of either the wild-type MMLV (SEQ ID NO 15) or in a known MMLV variant (SEQ ID NO: 1). As used herein, a “mutation” refers to a change introduced into a parental or wild type DNA sequence that changes the amino acid sequence encoded by the DNA, including, but not limited to, substitutions, insertions, deletions, point mutations, mutation of multiple nucleotides or amino acids, transposition, inversion, frame shift, nonsense mutations, truncations or other forms of aberration that differentiate the polynucleotide or protein sequence from that of a wild-type sequence of a gene or gene product. The consequences of a mutation include, but are not limited to, the creation of a new character, property, function, or trait not found in the protein encoded by the parental DNA, including, but not limited to, N terminal truncation, C terminal truncation or chemical modification. A “mutation” also includes an N- or C-terminal extension. In some embodiments, the mutations disclosed herein are substitutions.

In particular, the present disclosure relates to mutant or modified reverse transcriptases that comprise one or more (e.g., one, two, three, four, five, ten, twelve, fifteen, twenty, etc.) amino acid changes. These amino acid changes render the reverse transcriptase more efficiency for nucleic acid synthesis (e.g., single cell profiling assay) requiring very small volume, as compared to an unmutated or an unmodified reverse transcriptase. As will be appreciated by those skilled in the art, one or more of the amino acids identified may be deleted and/or replaced with one or a number of amino acid residues. In a preferred aspect, any one or more of the amino acids may be substituted with any one or more amino acid residues such as Ala, Arg, Asn, Asp, Cys, Gln, Glu, Gly, His, He, Leu, Lys, Met, Phe, Pro, Ser, Thr, Trp, Tyr, and/or Val.

In some embodiments, the amino acid sequence of the engineered reverse transcriptase comprises E69K, L139P, D200N, E302R, T306K, W313F, T330P, N454K, H503V, D524N, L603W, E607K, and H634Y in SEQ ID NO: 15. In some embodiments, the amino acid sequence of the engineered reverse transcriptase comprises M66L, E69K, L139P, D200N, E302R, T306K, W313F, T330P, N454K, H503V, D524N, L603W, E607K, L435G and H634Y in SEQ ID NO: 15; and further comprises a combination of mutations M66L and. In some embodiments, the amino acid sequence of the engineered reverse transcriptase comprises E69K, L139P, D200N, E302R, T306K, W313F, T330P, N454K, H503V, D524N, L603W, E607K, H634Y M39V, M66L, and L435K in SEQ ID NO: 15. In some embodiments, the amino acid sequence of the engineered reverse transcriptase comprises M39V, E69K, L435K, L139P, D200N, E302R, T306K, W313F, T330P, N454K, H503V, D524N, L603W, E607K, and H634Y in SEQ ID NO: 15.

In some embodiments, the amino acid sequence of the engineered reverse transcriptase comprises E69K, L139P, D200N, E302R, T306K, W313F, T330P, N454K, H503V, D524N, L603W, E607K, H634Y, M66L, L435G, P448A and D449G in SEQ ID NO: 15. In some embodiments, the amino acid sequence of the engineered reverse transcriptase comprises E69K, L139P, D200N, E302R, T306K, W313F, T330P, N454K, H503V, D524N, L603W, E607K, M39V, M66L, L435G, P448A, D449G and H634Y in SEQ ID NO: 15. In some embodiments, the amino acid sequence of the engineered reverse transcriptase comprises M66L, E69K, L139P, D200N, E302R, T306K, W313F, T330P, N454K, H503V, D524N, L603W, E607K, and H634Y in SEQ ID NO: 15.

In some embodiments, the amino acid sequence of the engineered reverse transcriptase comprises E69K, L139P, D200N, E302R, T306K, W313F, T330P, L435G, P448A, D449G, N454K, D524N, L603W, and E607K in SEQ ID NO: 15. In some embodiments, the amino acid sequence of the engineered reverse transcriptase comprises M66L, E69K, L139P, D200N, E302R, T306K, W313F, T330P, L435G, P448A, D449G, N454K, D524N, L603W, and E607K in SEQ ID NO: 15.

In some embodiments, the amino acid sequence of the engineered reverse transcriptase comprises E69K, L139P, D200N, E302R, T306K, W313F, T330P, L435G, P448A, D449G, N454K, D524N, L603W, E607K, M66L, and H503V in SEQ ID NO: 15. In some embodiments, the amino acid sequence of the engineered reverse transcriptase comprises E69K, L139P, D200N, E302R, T306K, W313F, T330P, L435G, P448A, D449G, N454K, D524N, L603W, E607K, M66L and H634Y in SEQ ID NO: 15. In some embodiments, the amino acid sequence of the engineered reverse transcriptase comprises E69K, L139P, D200N, E302R, T306K, W313F, T330P, L435G, P448A, D449G, N454K, D524N, L603W, E607K, M66L, H503V, and H634Y in SEQ ID NO: 15.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671P in SEQ ID NO: 15.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, D524N, T542D, P627S, A644V, D653H, and K658R mutation in SEQ ID NO: 15. In that embodiment the D200 mutation is a D200N mutation, the D449 mutation is a D449G, the L603 mutation is an L603W, and the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, D524N, T542D, A644V, D653H, an R650H and K658R in SEQ ID NO: 15. In that embodiment, the D200 mutation is a D200N mutation, the D449 mutation is a D449E mutation, the L603 mutation is an L603W mutation, and the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, E545G, D583N, and H594Q in SEQ ID NO: 15. In that embodiment, the D200 mutation is a D200N mutation, the D449 mutation is a D449G mutation, the L603 mutation is an L603F mutation, and the E607 mutation is an E607K mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, D524N, T542D, A644V, D653H, and K658R in SEQ ID NO: 15. In that embodiment, the D200 mutation is a D200N mutation, the D449 mutation is a D449E mutation, the L603 mutation is an L603W mutation, the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, H204R, D524N, T542D, P627S, D583N, A644V, D653H and K658R in SEQ ID NO: 15. In that embodiment, the D200 mutation is a D200E mutation, the D449 mutation is a D449G mutation, the L603 mutation is an L603W mutation, the E607 mutation is an E607G mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, L671P, H204R, E545G, D583N, and H594Q in SEQ ID NO: 15. In that embodiment, the D200 mutation is a D200E mutation, the D449 mutation is a D449G mutation, the L603 mutation is an L603F mutation, the E607 mutation is an E607K mutation.

In some embodiments, the amino acid sequence of the engineered reverse transcriptase comprises M39V, E69K, L139P, a D200 mutation, E302R, T306K, W313F, T330P, G429S, P448A, a D449 mutation, L435K, N454K, a L603 mutation, a E607 mutation, and L671PP47L, D524N, T542D, D583N, P627S, A644V, D653H, and K658R in SEQ ID NO: 15. In that embodiment, the D200 mutation is a D200N mutation, the D449 mutation is a D449G mutation, the L603 mutation is an L603W mutation, the E607 mutation is an E607G mutation.

Some variants share the following alterations the combination of variants including T542D, D583N, E607G, A644V, D653H, and K658R (all relative to SEQ ID NO:15). Some variants share the following alterations the combination of variants including E545G, D583N, H594Q, and L603F (all relative to SEQ ID NO:15). These variants may further comprise additional alterations that may affect one or more reverse transcriptase related activities.

One aspect of the present disclosure provides an engineered reverse transcriptase comprising an amino acid sequence selected from the group consisting of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:30, SEQ ID NO:31, SEQ ID NO:32, SEQ ID NO:33, SEQ ID NO:34, SEQ ID NO:35, SEQ ID NO:36 and SEQ ID NO:37.

The percent sequence identity, in the context of two or more nucleic acid or polypeptide sequences, refers to the number of residues or bases that are the same for a given alignment of two polypeptide or nucleic acid sequences. Sequences sharing a specified percentage of nucleotides or amino acid residues, respectively, that are the same, when compared and aligned for a given parameter such as maximum correspondence, as measured using one of the sequence comparison algorithms described below (or other algorithms available to persons of skill) or by visual inspection.

By convention, amino acid additions, substitutions, and deletions within an aligned reference sequence are all differences that may reduce the percent identity depending upon the parameters used to assess percent identity. Often, additions, substitutions, and deletions within an aligned reference sequence are evaluated in an equivalent manner. In some cases, length variation between two sequences resulting in one sequence having bases or residues beyond the N- or C-terminus or 5′ or 3′ end of the other sequence are discarded in sequence alignment, such that the aligned region is defined by the ends of the shorter or earlier ending sequence and amino acids extending beyond the N- or C-terminus of a polynucleotide or 5′ or 3′ end of the earlier terminating sequence have no effect on percent identity scoring for aligned regions. For example, by one calculation approach, alignment of a 105 amino acid long polypeptide to a reference sequence 100 amino acids long would have a 100% identity score if the reference sequence fully was contained as a consecutive ungapped segment within the longer polynucleotide with no amino acid differences. Under such an assessment, a single amino acid difference (addition, deletion or substitution) between the two sequences within the 100-amino acid span of the aligned reference sequence would mean the two sequences were 99% identical.

In contrast, “substantially identical,” in the context of two nucleic acids or polypeptides (e.g., DNAs encoding a polymerase, or the amino acid sequence of a polymerase) refers to two or more sequences or subsequences that have at least about 60%, at least about 80%, at least about 90-95%, at least about 98%, at least about 99% or more nucleotide or amino acid residue identity, when compared and aligned for maximum correspondence, as measured using a sequence comparison algorithm, or by visual inspection. Such “substantially identical” sequences are typically considered to be “homologous,” without reference to actual ancestry. The “substantial identity” exists over a region of the sequences that is at least about 50 residues in length, at least about 100 residues, at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity over about 50, about 100, about 150 or more residues is routinely used to establish homology. Higher levels of sequence similarity, e.g., at least about 30%, about 40%, about 50%, about 60%, about 70%, about 80%, about 90%, about 95%, or about 99% or more, can also be used to establish homology.

Methods for determining sequence similarity percentages (e.g., BLAST protein (BLASTP) and nucleotide (BLASTN) using default parameters) are described herein and are generally available. For sequence comparison and homology determination, typically one sequence acts as a reference sequence to which test sequences are compared. When using a sequence comparison algorithm, test and reference sequences can be input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison are known to those skilled in the art.

In some embodiments, the engineered reverse transcriptase comprises an amino acid sequence that is at least about or about 90%, at least about or about 91%, at least about or about 92%, at least about or about 93%, at least about or about 94%, at least about or about 95%, at least about or about 96%, at least about or about 97%, at least about or about 98%, or at least about or about 99%, to an amino acid sequence selected from the group consisting of: SEQ ID NO: 1, SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, SEQ ID NO:13, SEQ ID NO: 14, SEQ ID NO: 15, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:30, SEQ ID NO:31, SEQ ID NO:32, SEQ ID NO:33, SEQ ID NO:34, SEQ ID NO:35, SEQ ID NO:36 and SEQ ID NO:37.

Another aspect of the present disclosure provides an engineered reverse transcriptase comprising the amino acid sequence of SEQ ID NO: 15, and further comprising a combination of mutations selected from the group consisting of T542D, D583N, E607G, A644V, D653H, K658R, E545G, D583N, H594Q, and a L603F. In some embodiments, the engineered transcriptase comprises an amino acid sequence that is at least about or about 90%, at least about or about 91%, at least about or about 92%, at least about or about 93%, at least about or about 94%, at least about or about 95%, at least about or about 96%, at least about or about 97%, at least about or about 98%, or at least about or about 99%, identical to an amino acid sequence selected from the group consisting of SEQ ID NO: 8, SEQ ID NO: 9, SEQ ID NO: 10, SEQ ID NO: 11, SEQ ID NO: 12, SEQ ID NO: 13, and SEQ ID NO: 14. In some embodiments, the engineered transcriptase comprises an amino acid sequence selected from the group consisting of SEQ ID NO: 8, SEQ ID NO: 9, SEQ ID NO: 10, SEQ ID NO: 11, SEQ ID NO: 12, SEQ ID NO: 13, and SEQ ID NO: 14.

One aspect of the present disclosure provides an engineered reverse transcriptase comprising an amino acid sequence that is at least 90% identical to the amino acid sequence of SEQ ID NO: 2. Another aspect of the present disclosure provides an engineered reverse transcriptase comprising an amino acid sequence that is at least 90% identical to the amino acid sequence of SEQ ID NO: 24.

In some embodiments, the engineered reverse transcription enzyme comprises an amino acid sequence that is at least 95% identical to a reverse transcriptase having the amino acid sequence set forth in SEQ ID NO:1. In some embodiments, the engineered reverse transcription enzyme comprises an amino acid sequence that is at least 95% identical to SEQ ID NO: 1 and has at least one mutation selected from the group of the following mutations; M39V mutation, a P47L mutation, M66L mutation, an E69K mutation, an L139P mutation, a D200N mutation, an H204R mutation, an E302R mutation, a T306K mutation, a W313F mutation, a T330P mutation, an L435G mutation, a G429S mutation, an L435K mutation, a P448A mutation, a D449G mutation, a N454K mutation, an H503V mutation, a D524N mutation, a T542 mutation, an E545G mutation, a D583N mutation, an H594Q mutation, an L603W mutation, an E607K mutation, a P627S mutation, an H634Y mutation, an A644V mutation, an R650H mutation, a D653H mutation, a K658R mutation, and an L671P mutation; and the engineered reverse transcription enzyme exhibits an altered reverse transcriptase related activity.

In some embodiments, the application provides an engineered reverse transcriptase comprising an amino acid sequence that is at least 95% identical to SEQ ID NO: 1 and wherein the amino acid sequence of said engineered reverse transcriptase comprises a combination of mutations indexed to SEQ ID NO: 15 selected from the group comprising (a) an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; (b) an M66L mutation, an E69K mutation, an L139P mutation, a D200N mutation, an E302R mutation, a T306K mutation, a W313F mutation, a T330P mutation, a N454K mutation, a D524N mutation, an H503V mutation, an L603W mutation, an E607K mutation, and an H634Y mutation, and at least one mutation selected from the group comprising an L435G mutation, an L435K mutation, an M39V mutation, a P448A mutation and a D449G mutation; (c) an M39V mutation, an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation; and (d) an M39V mutation, an E69K mutation, an L139P mutation, a D200 mutation, an E302R mutation, a T306K mutation, a W313F mutation, a T330P mutation, an L435K mutation, a G429S mutation, a P448A mutation, a D449 mutation, an N454K mutation, an L603 mutation, an E607 mutation and an L671P mutation, wherein said D200 mutation is selected from the group consisting of D200N and D200E, wherein said D449 mutation is selected from the group consisting of D449G an D449E, wherein said L603 mutation is selected from the group consisting of L603W and L603F, wherein said E607 mutation is selected from the group consisting of E607G and E607K, and further comprising at least one mutation selected from the group consisting of P47L, H204R, D524N, T542D, E545G, D583N, H594Q, P627S, A644V, R650H, D653H, K658R, L671P, and S679P.

In some embodiments an engineered reverse transcriptase of the present application has an amino acid sequence that is at least 95% identical to SEQ ID NO:1 and wherein the amino acid sequence of said engineered reverse transcriptase comprises a combination of mutations indexed to SEQ ID NO:15 wherein the amino acid sequence of said engineered reverse transcriptase comprises a combination of mutations selected from the group consisting of: an E69K mutation, an L139P mutation, a D200N mutation, an E302R mutation, a T306K mutation, a W313F mutation, a T330P mutation, a N454K mutation, an H503V mutation, a D524N mutation, an L603W mutation, an E607K mutation, and an H634Y mutation and further comprising a second combination of mutations selected from the group consisting of: (a) an M66L mutation and an L534G mutation, (b) an M39V mutation, an M66L mutation and an L435K mutation, (c) an M39V mutation and an L435K mutation, (d) an M66L mutation, an L435G mutation, a P448 mutation, and D449G mutation, and (e) an M39V mutation, an M66L mutation, an L435G mutation, a P448 mutation and a D449G mutation.

In some embodiments an engineered reverse transcriptase of the present application has an amino acid sequence that is at least 95% identical to SEQ ID NO:1 and wherein the amino acid sequence of the engineered reverse transcriptase comprises a combination of mutations selected from the group consisting of: an M39V mutation, an E69K mutation, an L139P mutation, a D200 mutation, an E302R mutation, a T306K mutation, a W313F mutation, a T330P mutation, a G429S mutation a P448A mutation, a D449 mutation, an L435K mutation, a N454K mutation, an L603 mutation, an E607 mutation, and an L671P mutation and further comprising a second combination of mutations selected from the group consisting of: (a) a D524N mutation, a T542D mutation, an A644V mutation, a D653H mutation, a K658R mutation, a S679P mutation, and wherein said D200 mutation is a D200N mutation, said D449 mutation is a D449G, said L603 mutation is an L603W, said E607 mutation is an E607G mutation, and a P627S mutation; (b) a D524N mutation, a T542D mutation, an A644V mutation, a D653H mutation, an R650 mutation and a K658R mutation, and wherein said D200 mutation is a D200N mutation, said D449 mutation is a D449E mutation, said L603 mutation is an L603W mutation, and said E607 mutation is an E607G mutation; (c) an E545G mutation, a D583N mutation, an H594Q mutation, and an S679P mutation, and wherein said D200 mutation is a D200N mutation, said D449 mutation is a D449G mutation, said L603 mutation is an L603F mutation, said E607 mutation is an E607K mutation; (d) a D524N mutation, a T542D mutation, an A644V mutation, a D653H mutation, and a K658R mutation, and wherein said D200 mutation is a D200N mutation, said D449 mutation is a D449E mutation, said L603 mutation is an L603W mutation, said E607 mutation is an E607G mutation; (e) an H204R mutation, a D524N mutation, a T542D mutation, a D583N mutation, an A644V mutation, a D653H mutation, and a K658R mutation, wherein said D200 mutation is a D200E mutation, said D449 mutation is a D449G mutation, said L603 mutation is an L603W mutation, said E607 mutation is an E607G mutation, and a P627S mutation, (f) an H204R mutation, an E454G mutation, a D583N mutation, an H594Q mutation, and an S679P mutation, wherein said D200 mutation is a D200E mutation, said D449 mutation is a D449G mutation, said L603 mutation is an L603F mutation, said E607 mutation is an E607K mutation; and (g) a P47 mutation, a D524N mutation, a T542D mutation, a D583N mutation, an A644V mutation, a D653H mutation, a K658R mutation and an S679P mutation, wherein said P47 mutation is a P47L mutation, D200 mutation is a D200N mutation, said D449 mutation is a D449G mutation, said L603 mutation is an L603W mutation, said E607 mutation is an E607G mutation, and a P627S mutation. A variant may comprise a combination of mutations or alterations and may further comprise a second combination of mutations.

In some embodiments, an engineered reverse transcriptase of the present application has an amino acid sequence set forth in the group comprising SEQ ID NO:2, SEQ ID NO:3, SEQ ID NO:4, SEQ ID NO:5, SEQ ID NO:6, SEQ ID NO: 7, SEQ ID NO: 8, SEQ ID NO:9, SEQ ID NO:10, SEQ ID NO:11, SEQ ID NO:12, SEQ ID NO: 13, and SEQ ID NO:14.

In some embodiments, an engineered reverse transcriptase of the present application comprise an amino acid sequence set forth in Table 2.

TABLE 2
Sequences

| SEQ ID | | |
|---|---|---|
| NO | Description | Sequence |
| 1 | Engineered reverse | MTWLSDFPQAWAETGGMGLAVRQAPLIIPLK |
| | transcriptase | ATSTPVSIKQYPMSQKARLGIKPHIQRLLDQGI |
| | (MMLV RT | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 2 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 3 | MMLV RT Variant | MTWLSDFPQAWAETGGVGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIKAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 4 | MMLV RT Variant | MTWLSDFPQAWAETGGVGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIKAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 5 | MMLV RT Variant | MTWLSDFPQAWAETGGMGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIKAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 6 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 7 | MMLV RT Variant | MTWLSDFPQAWAETGGVGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 8 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFNEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAGRWLSKARMTHYQALLLDTDRVQ |
| | | FGPVVALNPATLLPLPEEGLQHNCLDILAEAH |
| | | GTRPDLTDQPLPDADHTWYTNGSSLLQEGQR |
| | | KAGAAVTDETEVIWAKALPAGTSAQRAELIA |
| | | LTQALKMAEGKKLNVYTNSRYAFATAHIHGE |
| | | IYRRRGWLTSGGKEIKNKDEILALLKALFLSK |
| | | RLSIIHCPGHQKGHSVEARGNRMAHQAARRA |
| | | AITETPDTSTLP |
| 9 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFNEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAERWLSKARMTHYQALLLDTDRVQ |
| | | FGPVVALNPATLLPLPEEGLQHNCLDILAEAH |
| | | GTRPDLTDQPLPDADHTWYTNGSSLLQEGQR |
| | | KAGAAVTDETEVIWAKALPAGTSAQRAELIA |
| | | LTQALKMAEGKKLNVYTDSRYAFATAHIHGE |
| | | IYRRRGWLTSGGKEIKNKDEILALLKALFLPK |
| | | RLSIIHCPGHQKGHSVEARGNHMAHQAARRA |
| | | AITETPDTSTLP |
| 10 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFNEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAGRWLSKARMTHYQALLLDTDRVQ |
| | | FGPVVALNPATLLPLPEEGLQHNCLDILAEAH |
| | | GTRPDLTDQPLPDADHTWYTDGSSLLQEGQR |
| | | KAGAAVTTETGVIWAKALPAGTSAQRAELIA |
| | | LTQALKMAEGKKLNVYTNSRYAFATAHIQGE |
| | | IYRRRGFLTSKGKEIKNKDEILALLKALFLPKR |
| | | LSIIHCPGHQKGHSAEARGNRMADQAARKAA |
| | | ITETPDTSTLP |
| 11 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFNEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAERWLSKARMTHYQALLLDTDRVQ |
| | | FGPVVALNPATLLPLPEEGLQHNCLDILAEAH |
| | | GTRPDLTDQPLPDADHTWYTNGSSLLQEGQR |
| | | KAGAAVTDETEVIWAKALPAGTSAQRAELIA |
| | | LTQALKMAEGKKLNVYTDSRYAFATAHIHGE |
| | | IYRRRGWLTSGGKEIKNKDEILALLKALFLPK |
| | | RLSIIHCPGHQKGHSVEARGNRMAHQAARRA |
| | | AITETPDTSTLP |
| 12 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFEEALRRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAGRWLSKARMTHYQALLLDTDRVQ |
| | | FGPVVALNPATLLPLPEEGLQHNCLDILAEAH |
| | | GTRPDLTDQPLPDADHTWYTNGSSLLQEGQR |
| | | KAGAAVTDETEVIWAKALPAGTSAQRAELIA |
| | | LTQALKMAEGKKLNVYTNSRYAFATAHIHGE |
| | | IYRRRGWLTSGGKEIKNKDEILALLKALFLSK |
| | | RLSIIHCPGHQKGHSVEARGNRMAHQAARRA |
| | | AITETPDTSTLP |
| 13 | MMLV RT Variant | TLNIEDEHRLHETSKEPDVSLGSTWLSDFPQA |
| | | WAETGGVGLAVRQAPLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFEEALRRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAGRWLSKARMTHYQALLLDTDRVQ |
| | | FGPVVALNPATLLPLPEEGLQHNCLDILAEAH |
| | | GTRPDLTDQPLPDADHTWYTDGSSLLQEGQR |
| | | KAGAAVTTETGVIWAKALPAGTSAQRAELIA |
| | | LTQALKMAEGKKLNVYTNSRYAFATAHIQGE |
| | | IYRRRGFLTSKGKEIKNKDEILALLKALFLPKR |
| | | LSIIHCPGHQKGHSAEARGNRMADQAARKAA |
| | | ITETPDTSTLP |
| 14 | MMLV RT Variant | TLNIEDEHRLHETSKEPDVSLGSTWLSDFPQA |
| | | WAETGGVGLAVRQALLIIPLKATSTPVSIKQY |
| | | PMSQKARLGIKPHIQRLLDQGILVPCQSPWNT |
| | | PLLPVKKPGTNDYRPVQDLREVNKRVEDIHPT |
| | | VPNPYNLLSGPPPSHQWYTVLDLKDAFFCLRL |
| | | HPTSQPLFAFEWRDPEMGISGQLTWTRLPQGF |
| | | KNSPTLFNEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLRRFLGKAGFCRLFIPGF |
| | | AEMAAPLYPLTKPGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMSQPLVIKAPHAVE |
| | | ALVKQPAGRWLSKARMTHYQALLLDTDRVQ |
| | | FGPVVALNPATLLPLPEEGLQHNCLDILAEAH |
| | | GTRPDLTDQPLPDADHTWYTNGSSLLQEGQR |
| | | KAGAAVTDETEVIWAKALPAGTSAQRAELIA |
| | | LTQALKMAEGKKLNVYTNSRYAFATAHIHGE |
| | | IYRRRGWLTSGGKEIKNKDEILALLKALFLSK |
| | | RLSIIHCPGHQKGHSVEARGNRMAHQAARRA |
| | | AITETPDTSTLP |
| 15 | Wild-type MMLV | TLNIEDEHRLHETSKEPDVSLGSTWLSDFPQA |
| | GenBank Seq ID | WAETGGMGLAVRQAPLIIPLKATSTPVSIKQY |
| | NP_955591.1 p80 | PMSQEARLGIKPHIQRLLDQGILVPCQSPWNT |
| | RT | PLLPVKKPGTNDYRPVQDLREVNKRVEDIHPT |
| | | VPNPYNLLSGLPPSHQWYTVLDLKDAFFCLRL |
| | | HPTSQPLFAFEWRDPEMGISGQLTWTRLPQGF |
| | | KNSPTLFDEALHRDLADFRIQHPDLILLQYVD |
| | | DLLLAATSELDCQQGTRALLQTLGNLGYRAS |
| | | AKKAQICQKQVKYLGYLLKEGQRWLTEARK |
| | | ETVMGQPTPKTPRQLREFLGTAGFCRLWIPGF |
| | | AEMAAPLYPLTKTGTLFNWGPDQQKAYQEIK |
| | | QALLTAPALGLPDLTKPFELFVDEKQGYAKG |
| | | VLTQKLGPWRRPVAYLSKKLDPVAAGWPPCL |
| | | RMVAAIAVLTKDAGKLTMGQPLVILAPHAVE |
| | | ALVKQPPDRWLSNARMTHYQALLLDTDRVQ |
| | | FGPVVALNPATLLPLPEEGLQHNCLDILAEAH |
| | | GTRPDLTDQPLPDADHTWYTDGSSLLQEGQR |
| | | KAGAAVTTETEVIWAKALPAGTSAQRAELIAL |
| | | TQALKMAEGKKLNVYTDSRYAFATAHIHGEI |
| | | YRRRGLLTSEGKEIKNKDEILALLKALFLPKRL |
| | | SIIHCPGHQKGHSAEARGNRMADQAARKAAI |
| | | TETPDTSTLL |
| 16 | Affinity Tag | His His His His His |
| 17 | a poly-dT sequence | TTTTTTTTTT |
| 18 | mRNA GAPDH | AGA GGG AAA GCA GCC CGC AGC CUC CCG |
| | | CUU CGC UCU CUG CUC CUC CUG UUC GAC |
| | | AGU CAG CCG CAU CUU C?U UUG CGU CGC |
| | | CAG CCG AGC CAC AUC GCU CAG ACA CCA |
| | | UGG GGA AGG UGA AGG UCG GAG UCA |
| | | ACG GAU UUG GUC GUA UUG GGC GCC |
| | | UGG UCA CCA GGG CUG CUU UUA ACU |
| | | CUG GUA AAG UGG AUA UUG UUG CCA |
| | | UCA AUG ACC CCU UCA UUG ACC UCA ACU |
| | | ACA UGG UUU ACA UGU UCC AAU AUG |
| | | AUU CCA CCC AUG GCA AAU UCC AUG GCA |
| | | CCG UCA AGG CUG AGA ACG GGA AGC |
| | | UUG UCA UCA AUG GAA AUC CCA UCA |
| | | CCA UCU UCC AGG AGC GAG AUC CCU CCA |
| | | AAA UCA AGU GGG GCG AUG CUG GCG |
| | | CUG AGU ACG UCG UGG AGU CCA CUG |
| | | GCG UCU UCA CCA CCA UGG AGA AGG |
| | | CUG GGG CUC AUU UGC AGG GGG GAG |
| | | CCA AAA GGG UCA UCA UCU CUG CCC CCU |
| | | CUG CUG AUG CCC CCA UGU UCG UCA UGG |
| | | GUG UGA ACC AUG AGA AGU AUG ACA |
| | | ACA GCC UCA AGA UCA UCA GCA AUG CCU |
| | | CCU GCA CCA CCA ACU GCU UAG CAC CCC |
| | | UGG CCA AGG UCA UCC AUG ACA ACU |
| | | UUG GUA UCG UGG AAG GAC UCA UGA |
| | | CCA CAG UCC AUG CCA UCA CUG CCA CCC |
| | | AGA AGA CUG UGG AUG GCC CCU CCG |
| | | GGA AAC UGU GGC GUG AUG GCC GCG |
| | | GGG CUC UCC AGA ACA UCA UCC CUG CCU |
| | | CUA CUG GCG CUG CCA AGG CUG UGG |
| | | GCA AGG UCA UCC CUG AGC UGA ACG |
| | | GGA AGC UCA CUG GCA UGG CCU UCC |
| | | GUG UCC CCA CUG CCA ACG UGU CAG UGG |
| | | UGG ACC UGA CCU GCC GUC UAG AAA |
| | | AAC CUG CCA AAU AUG AUG ACA UCA |
| | | AGA AGG UGG UGA AGC AGG CGU CGG |
| | | AGG GCC CCC UCA AGG GCA UCC UGG GCU |
| | | ACA CUG AGC ACC AGG UGG UCU CCU CUG |
| | | ACU UCA ACA GCG ACA CCC ACU CCU CCA |
| | | CCU UUG ACG CUG GGG CUG GCA UUG CCC |
| | | UCA ACG ACC ACU UUG UCA AGC UCA |
| | | UUU CCU GGU AUG ACA ACG AAU UUG |
| | | GCU ACA GCA ACA GGG UGG UGG ACC |
| | | UCA UGG CCC ACA UGG CCU CCA AGG AGU |
| | | AAG ACC CCU GGA CCA CCA GCC CCA GCA |
| | | AGA GCA CAA GAG GAA GAG AGA GAC |
| | | CCU CAC UGC UGG GGA GUC CCU GCC ACA |
| | | CUC AGU CCC CCA CCA CAC UGA AUC UCC |
| | | CCU CCU CAC AGU UGC CAU GUA G?C CCC |
| | | UUG AAG AGG GGA GGG GCC UAG GGA |
| | | GCC GCA CCU UGU CAU GUA CCA UCA AUA |
| | | AAG UAC CCU GUG CUC AAC C |
| 19 | Primer for GAPDH | /56-FAM/TGG TTG AGC ACA GGG TAC TTT |
| | | ATT GAT GG |
| 20 | TSO sequence | AAG CAG TGG TAT CAA CGC AGA GTA CAT |
| | | rGrGrG |
| 21 | missing | missing |
| 22 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 23 | MMLV RT Variant | MTWLSDFPQAWAETGGVGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 24 | MMLV RT Variant | MTWLSDFPQAWAETGGMGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 25 | MMLV RT Variant | MTWLSDFPQAWAETGGMGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAYFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPAGRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 26 | MMLV RT Variant | MTWLSDFPQAWAETGGVGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 27 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPADRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 28 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPGRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 29 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAVGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 30 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIYCPGHQKGHSAEARGNR |
| | | MADQAARKAAITETPDTSTLL |
| 31 | MMLV RT Variant | MTWLSDFPQAWAETGGMGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 32 | MMLV RT Variant | MTWLSDFPQAWAETGGMGLAVRQAPLIIPLK |
| | | ATSTPVSIKQYPMSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAYFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 33 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGGQKGHSAEARGNR |
| | | MADQAARKAAITETPDTSTLL |
| 34 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAYFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGGQKGHSAEARGNR |
| | | MADQAARKAAITETPDTSTLL |
| 35 | MMLV RT Variant | MTWLSDFPQAWAETGGVGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 36 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAYFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 37 | MMLV RT Variant | MTWLSDFPQAWAETGGMGLAVRQAPLIIPLK |
| | | ATSTPVSIKQYPLSQKARLGIKPHIQRLLDQGI |
| | | LVPCQSPWNTPLLPVKKPGTNDYRPVQDLRE |
| | | VNKRVEDIHPTVPNPYNLLSGPPPSHQWYTVL |
| | | DLKDAFFCLRLHPTSQPLFAFEWRDPEMGISG |
| | | QLTWTRLPQGFKNSPTLFNEALHRDLADFRIQ |
| | | HPDLILLQYVDDLLLAATSELDCQQGTRALLQ |
| | | TLGNLGYRASAKKAQICQKQVKYLGYLLKEG |
| | | QRWLTEARKETVMGQPTPKTPRQLRRFLGKA |
| | | GFCRLFIPGFAEMAAPLYPLTKPGTLFNWGPD |
| | | QQKAYQEIKQALLTAPALGLPDLTKPFELFVD |
| | | EKQGYAKGVLTQKLGPWRRPVAYLSKKLDP |
| | | VAAGWPPCLRMVAAIAVLTKDAGKLTMGQP |
| | | LVIGAPHAVEALVKQPPDRWLSKARMTHYQ |
| | | ALLLDTDRVQFGPVVALNPATLLPLPEEGLQH |
| | | NCLDILAEAHGTRPDLTDQPLPDADHTWYTN |
| | | GSSLLQEGQRKAGAAVTTETEVIWAKALPAG |
| | | TSAQRAELIALTQALKMAEGKKLNVYTDSRY |
| | | AFATAHIHGEIYRRRGWLTSKGKEIKNKDEIL |
| | | ALLKALFLPKRLSIIHCPGHQKGHSAEARGNR |
| | | MADQAARKAAITETPDTSTLL |
| 38 | a histidine | HHHHHH |
| | purification tag | |
| 39 | short peptide C- | SEEDEEKEEDG |
| | terminal tag | |
| 40 | Tobacco etch virus | ENLYFQ/G |
| | protease (TEV) | |
| | cleavage site | |
| 41 | Enterokinase (EntK) | DDDDK/ |
| | cleavage site | |
| 42 | Factor Xa (Xa) | IEGR/ |
| | cleavage site | |
| 43 | Thrombin (Thr) | LVPR/GS |
| | cleavage site | |
| 44 | Genetically | LEVLFQ/GP |
| | engineered | |
| | derivative of human | |
| | rhinovirus 3C | |
| | protease cleavage | |
| | site | |

### 1. Tag

One aspect of the present disclosure provides an engineered reverse transcriptase enzyme comprising a tag protein. Tags used in the practice of the invention may serve any number of purposes and a number of tags may be added to impart one or more different functions to the engineered reverse transcriptase, and/or derivatives thereof, of the disclosure. For example, tags may (1) contribute to protein-protein interactions both internally within a protein and with other protein molecules, (2) make the protein amenable to particular purification methods, (3) enable one to identify whether the protein is present in a composition; or (4) give the protein other functional characteristics.

In some embodiments, the engineered reverse transcriptase described herein further comprises a tag protein selected from the group consisting of an affinity tag, a fluorescent tag, or an expression and/or solubility enhancement tag. In some embodiments, the tag protein is selected from hexahistidine tag (his-tag), Fasciola hepatica 8-kDa antigen tag (Fh8), Glutathione-S-transferase (GST) tag, maltose-binding protein tag (MBP), FLAg tag peptide (FLAG tag), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin), small ubiquitin-like modifier tag (SUMO), a strep tag, Thioredoxin (Trx) tag, aVariFlex C-Terminal solubility enhancement tag, a short peptide C-terminal tag, Solubility-enhancer peptide sequences (SET) tag, IgG domain B1 of Protein G (GB1) tag, IgG repeat domain ZZ of Protein A (ZZ) tag, Mutated dehalogenase tag (HaloTag), Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Stress-responsive proteins tag (e.g., RpoA, tag, SlyD Tsf tag, RpoS tag, PotD tag, or Crr tag), and E. coli acidic proteins tag (e.g., msyB tag, yigD tag, and rpoD tag). Additional affinity tags and solubility enhancer tags are known to those skill in the art. See Costa et al., Front. Microbiol., 63(5): (2014); Esposito and Chatterjee Curr. Opin. Biotechnol., 17: 353-358 (2006); Malhotra, A. “Tagging for protein expression,” in Guide to Protein Purification, 2nd Edn, eds. R. R. Burgess and M. P. Deutscher (San Diego, CA: Elsevier), 463:239-258 (2009).

In some embodiments, the tag is selected from hexahistidine tag (his-tag), small ubiquitin-like modifier tag (SUMO), aVariFlex C-Terminal solubility enhancement tag, a short peptide C-terminal tag, Thioredoxin (Trx) tag, aVariFlex C-Terminal solubility enhancement tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), or fungal avidin-like protein (Tamavidin).

In one embodiment, the tag is an affinity tag selected from a histidine tag such as a hexahistidine tag (his-tag or 6 His-tag), Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin). In one embodiment, the tag is a hexahistidine tag.

In some embodiments, the tag is selected from a small ubiquitin-like modifier tag (SUMO), a VariFlex C-Terminal solubility enhancement tag, a short peptide C-terminal tag, Thioredoxin (Trx) tag, Solubility-enhancer peptide sequences (SET) tag, IgG domain B1 of Protein G (GB1) tag, IgG repeat domain ZZ of Protein A (ZZ) tag, Solubility enhancing Ubiquitous Tag (SNUT tag), Seventeen kilodalton protein (Skp tag), Phage T7 protein kinase (T7PK) tag, E. coli secreted protein A (EspA) tag, Monomeric bacteriophage T7 0.3 protein (Orc protein) (Mocr) tag, E. coli trypsin inhibitor (Ecotin) tag, Calcium-binding protein (CaBP) tag, Stress-responsive arsenate reductase (ArsC) tag, N-terminal fragment of translation initiation factor IF2 (IF2-domain I) tag, N-terminal fragment of translation initiation factor IF2 (Expressivity) tag, Fasciola hepatica 8-kDa antigen tag (Fh8), Glutathione-S-transferase (GST) tag, maltose-binding protein tag (MBP), FLAg tag peptide (FLAG), streptavidin binding peptide tag (Strep-II; strep), calmodulin-binding protein tag (CBP), mutated dehalogenase tag (HaloTag), staphylococcal Protein A (Protein A), intein mediated purification with the chitin-binding domain (IMPACT (CBD)), cellulose-binding module (CBM), dockerin domain of Clostridium josui tag (Dock), fungal avidin-like protein (Tamavidin).

In some embodiments, the solubility enhancer tag is selected from the group consisting of a SUMO tag, a GST tag, a Trx tag, a VariFlex C-Terminal solubility enhancement tag, a short peptide C-terminal tag, an Fh8 tag, MBP tag, SET tag, GB1 tag, ZZ tag, HaloTag, SNUT tag, Skp tag, T7PK tag, EspA tag, Mocr tag, Ecotin tag, CaBO tag, ArsC tag, IF2-domain I tag, Expressivity tag, RpoA, tag, SlyD, tag, Tsf tag, RpoS tag, PotD tag, Crr tag, msyB tag, yigD tag, and rpoD tag.

In some embodiments, the tag is an affinity tag. In one embodiment, the tag is an affinity tag and comprises a histidine purification tag. In one embodiment, the tag is a hexahistidine tag (his tag). In one embodiment, the tag comprises an amino acid sequence of the sequence HHHHHH (SEQ ID NO: 38). In one embodiment, the tag is a solubility enhancer tag. In one embodiment, the solubility enhancer tag is a short peptide C-terminal tag. In one embodiment, the solubility enhancer tag comprises an amino acid sequence of SEEDEEKEEDG (SEQ ID NO: 39) or an amino acid sequence having at least 90% sequence identity to the amino acid sequence of SEQ ID NO: 39.

In some embodiments, the engineered transcriptase enzyme or derivatives thereof comprises an affinity tag at the N-terminus or at the C-terminus of the amino acid sequence. In some embodiments, the affinity tag include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity eXact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, the affinity tag is at least 5 histidine amino acids.

In some embodiments, the engineered reverse transcription enzyme may include an affinity tag at the N-terminus or at the C-terminus of the amino acid sequence. In some embodiments, an affinity tag be cleaved from the reverse transcriptase enzyme prior to use, or it may remain on the reverse transcriptase wherein said inclusion does not alter appreciably the reverse transcriptase's activity. In some instances, the affinity tag may include, but is not limited to, albumin binding protein (ABP), AU1 epitope, AU5 epitope, T7-tag, V5-tag, B-tag, Chloramphenicol Acetyl Transferase (CAT), Dihydrofolate reductase (DHFR), AviTag, Calmodulin-tag, polyglutamate tag, E-tag, FLAG-tag, HA-tag, Myc-tag, NE-tag, S-tag, SBP-tag, Doftag 1, Softag 3, Spot-tag, tetracysteine (TC) tag, Ty tag, VSV-tag, Xpress tag, biotin carboxyl carrier protein (BCCP), green fluorescent protein tag, HaloTag, Nus-tag, thioredoxin-tag, Fc-tag, cellulose binding domain, chitin binding protein (CBP), choline-binding domain, galactose binding domain, maltose binding protein (MBP), Horseradish Peroxidase (HRP), Strep-tag, HSV epitope, Ketosteroid isomerase (KSI), KT3 epitope, LacZ, Luciferase, PDZ domain, PDZ ligand, Polyarginine (Arg-tag), Polyaspartate (Asp-tag), Polycysteine (Cys-tag), Polyphenylalanine (Phe-tag), Profinity exact, Protein C, S1-tag, S1-tag, Staphylococcal protein A (Protein A), Staphylococcal protein G (Protein G), Small Ubiquitin-like Modifier (SUMO), Tandem Affinity Purification (TAP), TrpE, Ubiquitin, Universal, glutathione-S-transferase (GST), and poly(His) tag. In some instances, said affinity tag is at least 5 histidine amino acids (SEQ ID NO: 16).

In some embodiments, the tag further comprises an endoprotein cleavage site selected from ENLYFQ/G (SEQ ID NO: 40), DDDDK/(SEQ ID NO: 41), IEGR/(SEQ ID NO: 42), LVPR/GS (SEQ ID NO: 43), or LEVLFQ/GP (SEQ ID NO: 44).

One of skill will recognize that modifications can additionally be made to the polymerases of the present invention without diminishing their biological activity. Some modifications may be made to facilitate the cloning, expression, or incorporation of a domain into a fusion protein. Such modifications are well known to those of skill in the art and include, for example, the addition of codons at either terminus of the polynucleotide that encodes the binding domain to provide, for example, a methionine added at the amino terminus to provide an initiation site, or additional amino acids placed on either terminus to create conveniently located restriction sites or termination codons or purification sequences.

One or more of the domains may also be modified to facilitate the linkage of a variant reverse transcriptase to another molecule to obtain polynucleotides. Thus, engineered reverse transcriptase that are modified by such methods are also part of the invention. For example, a codon for a cysteine residue can be placed at either end of a reverse transcriptase so that the reverse transcriptase can be linked by, for example, a sulfide linkage. The modification can be performed using either recombinant or chemical methods (see e.g., Pierce Chemical Co. catalog, Rockford IL).

### 2. Protease Cleavage Sequence

In some embodiments, the engineered reverse transcriptase enzyme or a derivative thereof further comprises a protease cleavage sequence. In some embodiments, the cleavage of the protease cleavage sequence by a protease results in cleavage of the affinity tag from the engineered reverse transcriptase enzyme or a derivative thereof. In some instances, the protease cleavage sequence/site is recognized by a protease including, but not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase (EnTK), gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin (Thr), tissue kallikrein, tobacco etch virus (TEV), togavirin, tryptophanyl aminopeptidase, U-plasminogen activator, V8, venombin A, venombin AB, factor Xa (Xa), and Xaa-pro aminopeptidase. In some embodiments, the protease cleavage sequence is a thrombin cleavage sequence.

In some embodiments, the engineered reverse transcriptase enzyme or a derivative thereof disclosed herein comprises an amino acid sequence of ENLYFQ/G (SEQ ID NO: 40), DDDDK/(SEQ ID NO: 41), IEGR/(SEQ ID NO: 42), LVPR/GS (SEQ ID NO: 43), or LEVLFQ/GP (SEQ ID NO: 44).

In some embodiments, the tag is cleaved or removed from the engineered reverse transcriptase enzyme or derivatives thereof via the cleavage site. In one embodiment, the tag is cleaved or removed using an endoprotein selected from the group consisting of tobacco etch virus protease (Tev), enterokinase (EntK), factor Xa (Xa), thrombin (Thr), genetically engineered derivative of human rhinovirus 3C protease (PreScission), Catalytic core of Ulp1 (SUMO protease). In one embodiment, the tag is cleaved at ENLYFQ/G (SEQ ID NO: 40) using tobacco etch virus protease (Tev). In another embodiment, the tag is cleaved at DDDDK/(SEQ ID NO: 41) using Enterokinase (EntK). In another embodiment, the tag is cleaved at IEGR/(SEQ ID NO: 42) using Factor Xa (Xa). In another embodiment, the tag is cleaved at LVPR/GS (SEQ ID NO: 43) using thrombin (Thr). In another embodiment, the tag is cleaved at LEVLFQ/GP (SEQ ID NO: 44) using a genetically engineered derivative of human rhinovirus 3C protease. In another embodiment, the tag is cleaved with Catalytic core of Ulp1 (SUMO protease). Catalytic core of Ulp1 recognizes SUMO tertiary structure and cleaves at the C-terminal end of the conserved Gly-Gly sequence in SUMO.

In some embodiments, an engineered reverse transcription enzyme of the present disclosure further comprises a protease cleavage sequence, wherein cleavage of the protease cleavage sequence by a protease results in cleavage of the affinity tag from the engineered reverse transcription enzyme. In some instances, a protease cleavage sequence includes, but is not limited to, alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase. In some instances, the protease cleavage sequence is a thrombin cleavage sequence.

### B. Enhanced Reverse Transcriptase Activity

The engineered reverse transcriptase of the present disclosure is a variant Moloney Murine Leukemia Virus (MMLV) reverse-transcriptase with increased or enhanced reverse transcriptase activity. The term “increased” reverse transcriptase activity refers to the level of reverse transcriptase activity of a variant (e.g., mutant reverse transcriptase enzyme (e.g, MMLV variants disclosed herein) as compared to its wild-type form (e.g., wt MMLV or MMLV having the amino acid of SEQ ID NO: 15) or a known variant (e.g., MMLV having the amino acid of SEQ ID NO: 1). A mutant enzyme is said to have an “increased” reverse transcriptase activity if the level of its reverse transcriptase activity (as measured by methods described herein or known in the art) is at least 10% or more than its wild-type or a known variant. For example, the variant can have at least 10%, 15%, 20%, 25%, 30%, 40%, 50%, 60%, 70%, 80%, 90%, 100% more or at least 2-fold, 3-fold, 4-fold, 5-fold, or 10-fold or more activity than the wild-type or known variant.

Reverse transcriptases of the invention include any reverse transcriptase having one or a combination of the properties described herein. Such properties include, but are not limited to, enhanced stability, enhanced thermostability, reduced or eliminated RNase H activity, reduced terminal deoxynucleotidyl transferase activity, increased accuracy, increased processivity, increased specificity and/or increased fidelity.

An engineered reverse transcriptase may exhibit one or more reverse transcriptase related activities including but not limited to, RNA-dependent DNA polymerase activity, RNAse H activity, DNA-dependent DNA polymerase activity, RNA binding activity, DNA binding activity, polymerase activity, primer extension activity, strand-displacement activity, helicase activity, strand transfer activity, template binding activity, and transcription template switching activity. It is recognized that a change in any activity may increase, decrease or have no effect on a different reverse-transcriptase related activity. It is also recognized that a change in one activity may alter multiple properties of a reverse transcriptase. It is understood that when multiple properties are affected, the properties may be altered similarly or differently. It is further recognized that methods of evaluating reverse transcriptase related activities are known in the art.

In some embodiments, the engineered reverse transcriptase possesses one or more of the following characteristics when compared to a wild-type reverse transcriptase or a reverse transcriptase comprising the amino acid of SEQ ID NO: 1: increased thermostability; increased thermoreactivity; increased resistance to reverse transcriptase inhibitors; increased ability to reverse transcribe difficult templates; increased speed; increased processivity; increased specificity; enhanced polymerization activity; or increased sensitivity.

In some embodiments, the increase in thermoreactivity, resistance to reverse transcriptase inhibitors, ability to reverse transcribe difficult templates, speed, processivity, specificity, or sensitivity of the engineered reverse transcriptase is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type reverse transcriptase or a reverse transcriptase comprising the amino acid of SEQ ID NO: 1. In Some embodiments, the polymerization activity of the engineered reverse transcriptase is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to a wild-type reverse transcriptase or a reverse transcriptase comprising the amino acid of SEQ ID NO: 1.

In some embodiments, the engineered reverse transcriptase exhibits an enhanced reverse transcriptase activity as compared to a reverse transcriptase having the amino acid sequence set forth in SEQ ID NO: 1 or 15. In some embodiments, the enhanced reverse transcriptase activity is selected from the group consisting of processivity, template switching efficiency, binding affinity, and transcription efficiency. In some embodiments, the enhanced reverse transcriptase activity is an enhanced template switching (TS) efficiency as compared to the template switching efficiency of a reverse transcriptase having the amino acid sequence set forth in SEQ ID NO: 1 or 15.

In some embodiments, the enhanced reverse transcriptase activity is an enhanced transcription efficiency as compared to the transcription efficiency of a reverse transcriptase having the amino acid sequence set forth in SEQ ID NO: 1 or 15. In some embodiments, the enhanced reverse transcriptase activity is the enhanced transcription efficiency and template switching efficiency as compared to a reverse transcriptase having the amino acid sequence set forth in SEQ ID NO: 1 or 15.

In some embodiments, the enhanced reverse transcriptase activity is the increased binding affinity as compared to the binding affinity of a reverse transcriptase having the amino acid sequence set forth in SEQ ID NO: 1 or 15.

### 1. Thermostability

As used herein, the term “thermostable” generally refers to an enzyme, such as a reverse transcriptase (“thermostable reverse transcriptase”), which retains a greater percentage or amount of its activity after a heat treatment than is retained by the same enzyme having wild type thermostability, after an identical treatment. Thus, a reverse transcriptase having increased/enhanced thermostability may be defined as a reverse transcriptase having any increase in thermostability, preferably from about 1.2 to about 10,000 fold, from about 1.5 to about 10,000 fold, from about 2 to about 5,000 fold, or from about 2 to about 2000 fold, or any value in between these amounts, and retention of activity after a heat treatment sufficient to cause a reduction in the activity of a reverse transcriptase that is wild type for thermostability. In other aspects of the disclosure, the increase in thermostability can be greater than about 5 fold, greater than about 10 fold, greater than about 50 fold, greater than about 100 fold, greater than about 500 fold, or greater than about 1000 fold.

To determine the thermostability of the engineered reverse transcriptase of the present disclosure, the engineered reverse transcriptase can be compared to the corresponding wild type MMLV or a variant thereof (e.g., SEQ ID NO: 1) to determine the relative enhancement or increase in thermostability. For example, after a heat treatment at 60º C for 5 minutes, the engineered reverse transcriptase may retain approximately 90% of the activity present before the heat treatment, whereas wild type MMLV or MMLV variant (e.g., SEQ ID NO: 1) may retain 10% of its original activity. Likewise, after a heat treatment at 60° C. for 15 minutes, the engineered reverse transcriptase may retain approximately 80% of its original activity, whereas wild type MMLV or MMLV variant may have no measurable activity. Similarly, after a heat treatment at 60° C. for 15 minutes, the engineered reverse transcriptase may retain approximately 50%, approximately 55%, approximately 60%, approximately 65%, approximately 70%, approximately 75%, approximately 80%, approximately 85%, approximately 90%, or approximately 95% of its original activity, whereas wild type MMLV or MMLV variant may have no measurable activity or may retain 20%, 15%, 10%, or none of its original activity. In the first instance (i.e., after heat treatment at 60º C for 5 minutes), the reverse transcriptase would be said to be 9-fold more thermostable than the wild type reverse transcriptase (90% compared to 10%). Examples of conditions which may be used to measure thermostability of an enzyme such as reverse transcriptases are set out in further detail below and in the Examples.

The thermostability of a reverse transcriptase (e.g., engineered reverse transcriptase as described herein) can be determined, for example, by comparing the residual activity of a reverse transcriptase that has been subjected to a heat treatment, e.g., incubated at 60° C. for a given period of time, for example, five minutes, to a control sample of the same reverse transcriptase that has been incubated at room temperature for the same length of time as the heat treatment. One way the residual activity may be measured is by following the incorporation of a radiolabeled deoxyribonucleotide into an oligodeoxyribonucleotide primer using a complementary oligoribonucleotide template. For example, the ability of the reverse transcriptase to incorporate [α-³²P]-dGTP into an oligo-dG primer using a poly(riboC) template may be assayed to determine the residual activity of the reverse transcriptase. Methods for measuring residual activity of reverse transcriptase and polymerases are known by those of skill in the art. See e.g., Nikiforov, T. T., Anal Biochem., 2011, 412(2): 229-36, which is hereby incorporated by reference.

In some embodiments, the engineered reverse transcriptase enzyme of the present disclosure is thermophilic. In one embodiment, the engineered reverse transcriptase is resistant to thermal inactivation when compared to a wild-type polymerase. In another embodiment, the engineered reverse transcriptase is resistant to thermal inactivation at a temperature from about 53ºC to about 75° C.; from about 55° C. to about 75° C.; from about 60° ° C. to about 75° C.; from about 53ºC to about 68° C.; from about 55° C. to about 68° C.; from about 45° C. to about 68° C.; or from about 50° C. to about 68° C. In yet another embodiment, the engineered reverse transcriptase is resistant to thermal inactivation at a temperature of about 68° C.

In another embodiment, the thermostability of the engineered reverse transcriptase enzyme is determined by measuring the half-life of the engineered reverse transcriptase enzyme. Such half-life may be compared to a control or wild type enzyme to determine the difference (or delta) in half-life.

### 2. Half-Life

In some embodiments, the engineered reverse transcriptase enzyme possesses an enhanced half-life when compared to a wild-type polymerase and/or a wild-type reverse transcriptase at a temperature from about 53ºC to about 75° C.; from about 55° C. to about 75° C.; from about 60° ° C. to about 75° C.; from about 53° ° C. to about 68° C.; from about 55° C. to about 68° C.; from about 45° C. to about 68° C.; or from about 50° C. to about 68° C.

The half-life of the engineered reverse transcriptase enzyme of the disclosure is preferably determined at elevated temperatures (e.g., greater than 37° C.) and preferably at temperatures ranging from 40° C. to 80° C., or temperatures ranging from 45° C. to 75° C., 50° C. to 70° C., 55° C. to 65° C., and 58° C. to 62º C. Preferred half-lives of the engineered reverse transcriptase enzyme of the present disclosure may range from about 4 minutes to about 10 hours, about 4 minutes to about 7.5 hours, about 4 minutes to about 5 hours, about 4 minutes to about 2.5 hours, or about 4 minutes to about 2 hours, depending upon the temperature used. For example, the reverse transcriptase activity of the engineered reverse transcriptase of the present disclosure may have a half-life of at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 20 minute, at least about 25 minutes, at least about 30 minutes, at least about 40 minutes, at least about 50 minutes, at least about 60 minutes, at least about 70 minutes, at least about 80 minutes, at least about 90 minutes, at least about 100 minutes, at least about 115 minutes, at least about 125 minutes, at least about 150 minutes, at least about 175 minutes, at least about 200 minutes, at least about 225 minutes, at least about 250 minutes, at least about 275 minutes, at least about 300 minutes, at least about 400 minutes, at least about 500 minutes, or any time period in between these values, at temperatures of about 48° C., about 50° C., about 52º C, about 54º C, about 56° C., about 58° C., about 60° C., about 62° C., about 64° C., about 66° C., about 68° C., and/or about 70° C. In some embodiments, the thermostability of the engineered reverse transcriptase enzyme enhances the half-life of the engineered reverse transcriptase enzyme.

### 3. Processivity

In some embodiments, the engineered reverse transcriptase enzyme possesses one or more of the following characteristics when compared to a wild-type polymerase and/or reverse transcriptase: increased thermostability; increased thermoreactivity; increased resistance to reverse transcriptase inhibitors; increased ability to reverse transcribe difficult templates; increased speed; increased processivity; increased specificity; enhanced polymerization activity; increased sensitivity, or any combination thereof.

Processivity is defined as the ability of a polymerase or reverse transcriptase to carry out continuous nucleic acid synthesis on a template nucleic acid without frequent dissociation. It can be measured by the average number of nucleotides incorporated by a polymerase on a single association/disassociation event. DNA polymerase or reverse transcriptase alone produces short DNA product strand per binding event. Most DNA polymerases or reverse transcriptases are intrinsically low-processivity enzymes. The low processivity of DNA polymerase or reverse transcriptase alone is insufficient for the timely replication of a large genome.

In some embodiments, the polymerization activity of the engineered reverse transcriptase enzyme as described herein is enhanced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type reverse transcriptase.

In some embodiments, the engineered e reverse transcriptase enzyme reverse transcribes a RNA molecule having at least about 100, at least about 200, at least about 300, at least about 400, at least about 500, at least about 600, at least about 700, at least about 800, at least about 900, or at least about 1000 nucleotides. In another embodiment, the engineered reverse transcriptase enzyme reverse transcribes a RNA molecule that is at least about 1 kb, at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb, at least about 13 kb, at least about 14 kb, or at least about 15 kb. In another embodiment, the engineered reverse transcriptase enzyme reverse transcribes a RNA molecule that is at least about 7 kb or at least about 8 kb.

In some embodiments, the increase in thermoreactivity, resistance to reverse transcriptase inhibitors, ability to reverse transcribe difficult templates, speed, processivity, specificity, or sensitivity of the engineered reverse transcriptase enzyme as described herein has is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 90%, or about 100% as compared to the wild-type polymerase.

In some embodiments, the enhanced reverse transcriptase activity is an increased binding affinity and template switching efficiency as compared to a reverse transcriptase having the amino acid sequence set forth in SEQ ID NO:1. In some embodiments, the enhanced reverse transcriptase activity is an enhanced processivity as compared to the processivity of a reverse transcriptase having the amino acid sequence set forth in SEQ ID NO: 1.

### 4. Transcription Efficiency

In some embodiments, the engineered reverse transcriptase disclosed herein exhibits enhanced transcription efficiency when compared to the transcription efficiency of a reverse transcriptase having the amino acid sequence set forth in SEQ ID NO: 1 or SEQ ID NO: 15. As noted herein, the conversion of mRNA into cDNA by reverse transcriptase-mediated reverse transcription is an essential step in single cell profiling and gene expression analyses. However, the use of unmodified reverse transcriptase to catalyze reverse transcription is inefficient for all the reasons disclosed herein. The engineered reverse transcriptases of the disclosure are preferably modified or mutated such that the transcription efficiency of the engineered enzyme is increased or enhanced.

Furthermore, an engineered reverse transcription enzyme variants described herein may also exhibit unexpectedly higher resistance to cell lysate (i.e., are less inhibited by cell lysate) than that exhibited by an enzyme having the amino acid sequence set forth in SEQ ID NO:1. Lastly, an engineered reverse transcription enzyme variants of the present disclosure may have an unexpectedly greater ability to capture full-length transcripts (e.g., in T-cell receptor paired transcriptional profiling), as compared to that exhibited by an enzyme having the amino acid sequence set forth in SEQ ID NO:1.

It is recognized that a mutation of one or more residues may alter a first reverse transcriptase activity differently than a second reverse transcriptase activity. Further it is recognized that a different combination of mutations, such as different sites or residue changes may alter a reverse transcriptase activity similarly or differently. The variants that can template switch in the 5′ assay share the following alterations relative to SEQ ID NO:15: E69K, E302R, T306K, W313F, K435G, and N454K. These variants may further comprise additional alterations that may affect one or more reverse transcriptase related activities. Relative to SEQ ID NO:15, M39V and M66L may improve template switching. Without being limited by mechanism, variants comprising a M39V or a M66L mutation that do not exhibit altered performance in the 5′ GEM single cell assay may exhibit an altered processivity, an altered k_{d} or both. Relative to SEQ ID NO: 15, K435 mutants may improve thermostability in the presence of primer template. In the absence of primer template K435 variants may exhibit a thermal denaturation profile similar to that of the wild-type protein. Relative to SEQ ID NO:15, K435, P448 and D449 are residues in the connection domain; it was found that altering these residues may result in increased conformational flexibility. Additionally, the connection domain is thought to impact the conformational flexibility of the RNAase H domain. Relative to SEQ ID NO: 15, H503 and H634 occur within the RNAase H domain. The H503V and H634Y variants may impact primer-template contacting, processivity or both primer-template contacting and processivity.

The combination of variants including T542D, D583N, E607G, A644V, D653H, and K658R and the combination of variants including E545G, D583N, H594Q, and L603F may exhibit an altered RNAse H activity.

The engineered reverse transcription enzyme variants of the present disclosure unexpectedly provided an altered reverse transcriptase activity, such as but not limited to, improved thermal stability, processive reverse transcription, non-templated base addition, binding affinity, and template switching ability.

### 5. Template Switching Oligonucleotides

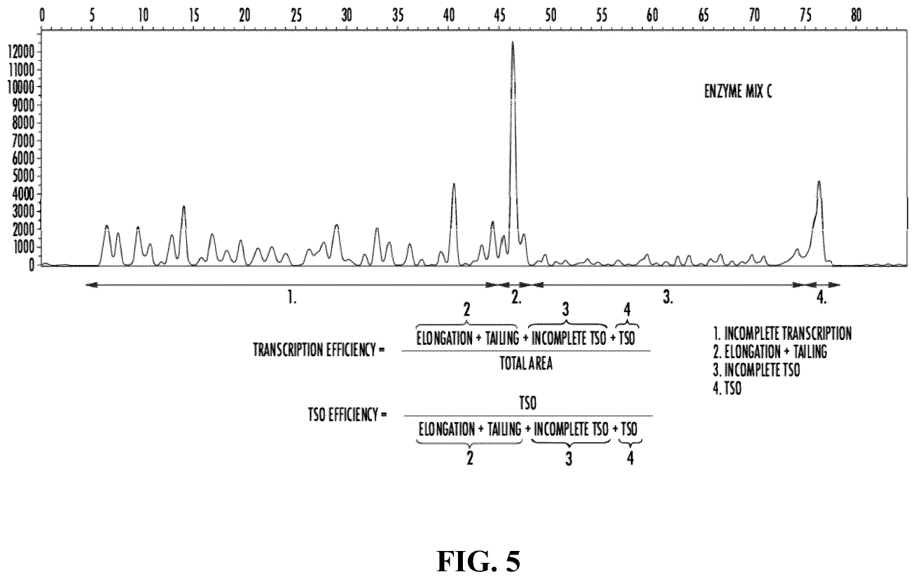

Transcription efficiency for a reverse transcription enzyme may be calculated as the sum of the area under the curve for the elongation and tailing (2), incomplete template switching (TSO) (3) and complete template switching (TSO) (4) regions over the total area under the curve for all products (FIG. 5). Transcription efficiency reflects all those products for which transcription was successfully completed. Template switching oligonucleotide efficiency may be calculated as the area under the curve for the complete template switching region (4) over the total area under the curve for all products including elongation and tailing (2), incomplete TSO (3) and complete TSO (4) (FIG. 5). An engineered reverse transcriptase may have an increased transcription efficiency, an increased TSO efficiency or both an increased transcription efficiency and an increased TSO efficiency.

For both transcription efficiency and template switching efficiency, lengths less than 45 nucleotides are considered incomplete (1). Lengths including the full length and the full length plus the tail are considered the elongation and tailing phase (2). Lengths longer than the full length plus the tail and shorter than the full length plus tail and template switching are considered incomplete template switching products (incomplete TSO, 3). Lengths having the full length plus tail and template switching size are considered template switched (TSO, 4).

Template switching oligonucleotides (also referred to herein as “switch oligos” or “switch oligonucleotides”) may be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as a template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination. Suitable lengths of a switch oligo are known in the art. See for example U.S. patent application Ser. No. 15/975,516 herein incorporated by reference in its entirety.

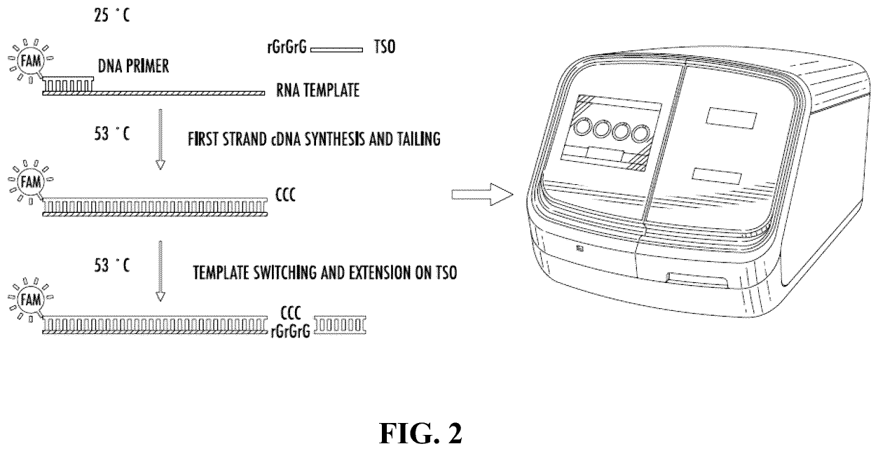

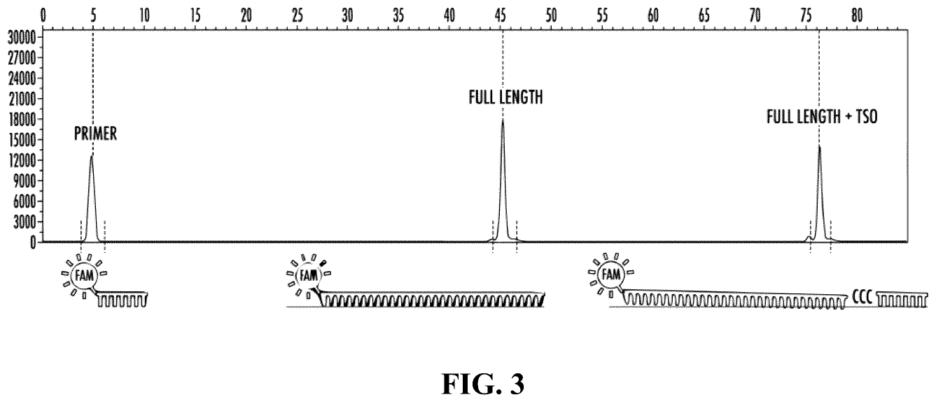

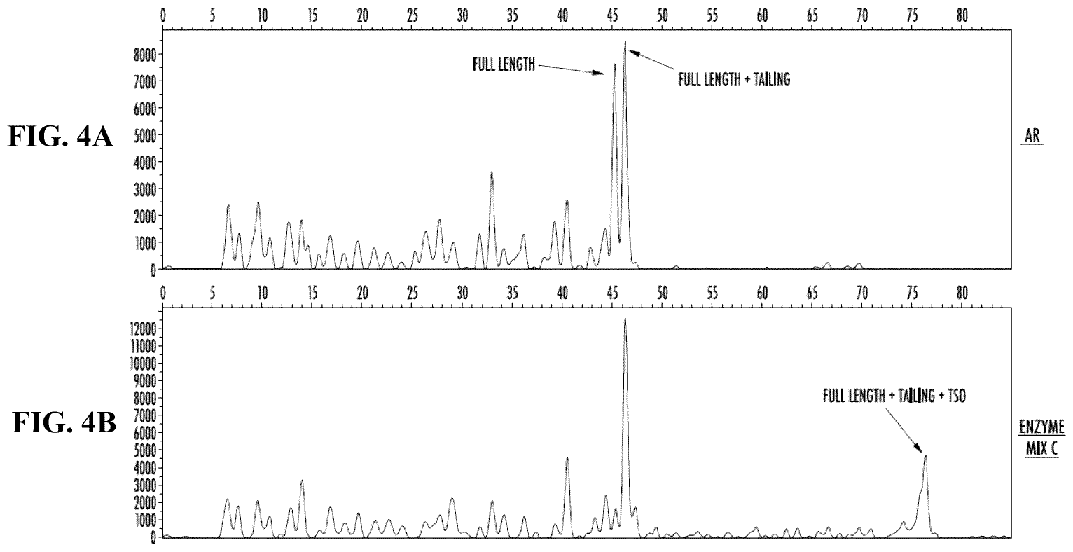

The general overview of template switching can be seen in FIG. 2. A primer can be hybridized to a RNA template, wherein the primer is extended by reverse transcription using a reverse transcriptase, thereby generating a first strand cDNA molecule. A polyC sequence can be added to the cDNA by a terminal transferase enzyme. A template switching oligonucleotide comprising a complementary polyG sequence to the polyC sequence added to the first strand cDNA, is added to the reaction, the polyG-TSO oligonucleotide hybridizes via complementarity to the polyC, and the reverse transcriptase can use that TSO sequence as a template for further extension. In the Examples, experiments for determining the efficiency of template switching are assayed on a capillary electrophoresis system such as a SeqStudio CE analyzer (ThermoFisher). Results from a CE assay, using fluorescently labelled polynucleotides, is exemplified in FIG. 3. With fluorescence on the Y axis and the nucleotide length on the X axis, a FAM labelled primer of 5 nt is shown, a FAM labelled first strand cDNA product of 45 nt is shown and a TSO extended first strand cDNA of approximately 75 nt is exemplified. FIG. 3 exemplifies this workflow showing experimental results using an RT enzyme known to have the ability to extend a polyG-TSO (enzyme C, or SEQ ID NO:1) compared to an RT that is not expected to extend a polyG-TSO (AR). On the top capillary electrophoretic graph, the full length cDNA product and a full length cDNA product with a TSO tail (tailing) are only approximately 1 nt different; no polyG TSO extension was generated. Conversely, using enzyme mix C that include SEQ ID NO:1 RT, the full length cDNA product and the full length cDNA product with efficient polyG-TSO extension were both generated.

An engineered reverse transcription enzyme of the current application may exhibit an altered base-biased template switching activity such as an increased base-biased template switching activity, decreased base-biased template switching activity or an altered base-bias to the template switching activity. An engineered reverse transcriptase variant may exhibit enhanced template switching with a 5′-G cap on the substrate.

### 6. RNase H Activity

In some embodiments, the engineered reverse transcription enzyme described herein is engineered to have reduced and/or abolished RNase activity. In some embodiments, the engineered reverse transcription enzyme engineered to have reduced and/or abolished RNase H activity comprises a mutation analogous to MMLV reverse transcriptase SEQ ID NO: 1 D561 mutation (SEQ ID NO:15 D583).

RNase H activity refers to endoribonuclease degradation of the RNA of a DNA-RNA hybrid to produce 5′ phosphate terminated oligonucleotides that are 2-9 bases in length. RNase H activity does not include degradation of single-stranded nucleic acids, duplex DNA or double-stranded RNA. Removal of the RNase H activity of reverse transcriptase can eliminate the problem of RNA degradation of the RNA template and improve the efficiency of reverse transcription.

In some embodiments, the reverse transcriptases of the present disclosure may have a reduced or substantially reduced RNase H activity. The reduction or substantial reduction or complete removal of the RNase H activity of a reverse transcriptase (e.g., MMLV) can prevent the degradation of an RNA template before the initiation of the RT reaction, thereby improving the efficiency of reverse transcription. See e.g., Gerard, et al., FOCUS 11(4):60 (1989); Gerard et al., FOCUS 14(3):91 (1992).

In some embodiments, the reverse transcriptases of the present disclosure substantially lacks RNase H activity. In that embodiment, the reverse transcriptases of the present disclosure have less than 10%, 5%, 1%, 0.5%, or 0.1% of the RNAse H activity of a wild type enzyme or a variant having the amino acid of SEQ ID NO: 1. In some embodiments, the reverse transcriptases of the present disclosure lacks RNase H activity. In that embodiment, the reverse transcriptases of the present disclosure have undetectable RNase H activity or have an RNase H activity that is less than about 1%, 0.5%, or 0.1% of the RNase H activity of a wild type enzyme or a variant comprising the amino acid of SEQ ID NO: 1.

As used herein, the term “reduced RNase H activity” means that the enzyme has less than 50%, e.g., less than 40%, 30%, or less than 25%, 20%, more preferably less than 15%, less than 10%, or less than 7.5%, and most preferably less than 5% or less than 2%, of the RNase H activity of the corresponding wild type enzyme or a variant comprising the amino acid of SEQ ID NO: 1. The RNase H activity of an enzyme may be determined by a variety of assays, such as those described, for example, in U.S. Pat. Nos. 5,405,776; 6,063,608; 5,244,797; and 5,668,005 in Kotewicz, M. L., et al., Nucl. Acids Res. 16:265 (1988) and Gerard, G. F., et al., FOCUS 14(5):91 (1992), the disclosures of all of which are fully incorporated herein by reference.

### III. Nucleic Acids and Expression Vectors

One aspect of the present disclosure provides an isolated nucleic acid molecule encoding the engineered reverse transcriptase or a derivatives thereof as described herein. In some embodiments, the engineered reverse transcriptase is encoded by a nucleic acid set forth herein or readily derived in light of polypeptide information provided herein (e.g., SEQ ID NO: 1-15, and 22-37) and known in the art. The engineered reverse transcriptases need not be encoded by any specific nucleic acid exemplified herein. For example, redundancy in the genetic code allows for variations in nucleotide codon sequences that nevertheless encode the same amino acid. Accordingly, engineered polymerases of the present disclosure can be produced from nucleic acid sequences that are different from those set forth herein, for example, being codon optimized for a particular expression system. Codon optimization can be carried out, for example, as set forth in Athey et al., BMC Bioinformatics, 18:391-401 (2017).

Wild type polymerase nucleic acids may be isolated from naturally occurring sources to be used as starting material to generate novel polymerases. Generally, the nomenclature and the laboratory procedures in recombinant DNA technology described below are those well known and commonly employed in the art. Standard techniques for cloning, DNA and RNA isolation, amplification and purification are known. Generally enzymatic reactions involving DNA ligase, DNA polymerase, restriction endonucleases are the like are performed according to the manufacturer's specifications. These techniques and various other techniques are generally performed according to Sambrook & Russell, Molecular Cloning-A Laboratory Manuel, Cold Spring Harbor Laboratory, Cold Spring Harbor, N.Y., (1989) or Ausubel et al., Current Protocols in Molecular Biology, Vol. 1-3, John Wiley & Sons, Inc. (1994-1998).

The isolation of polymerase nucleic acids may be accomplished by a variety of techniques. The polymerase nucleic acids of the present invention can be generated from the wild type sequences. The wild type sequences are altered to create modified sequences. Wild type polymerases can be modified to create the polymerases claimed in the present application using methods that are well known in the art. Exemplary modification methods are site-directed mutagenesis, point mismatch repair, or oligonucleotide-directed mutagenesis.

Another aspect of the present disclosure provides an expression vector comprising the isolated nucleic acid encoding the engineered reverse transcriptase or derivatives thereof as described herein. A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. The polymerases of the present disclosure can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, the following genera: Aspergillus, Trichoderma, Neurospora, Penicillium, Cephalosporium, Achlya, Podospora, Mucor, Cochliobolus, and Pyricularia. See, e.g., U.S. Pat. No. 5,679,543 and Stahl and Tudzynski, Eds., Molecular Biology in Filamentous Fungi, John Wiley & Sons, 1992. Synthesis of heterologous proteins in yeast is well known and described in the literature. Methods in Yeast Genetics, Sherman F. et al., Cold Spring Harbor Laboratory (1982) is a well recognized work describing the various methods available to produce the enzymes in yeast. There are many expression systems for producing the polymerase polypeptides of the present invention that are well know to those of ordinary skill in the art. See Gene Expression Systems, Fernandex and Hoeffler, Eds. Academic Press, 1999; Sambrook & Russell, supra; and Ausubel et al, Current Protocols in Molecular Biology, Vol. 1-3, John Wiley & Sons, Inc. (1994-1998).

Another aspect of the present disclosure provides a host cell transfected with the expression vector comprising the isolated nucleic acid encoding the engineered reverse transcriptase as described herein. Eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Once expressed, the engineered reverse transcriptase or a derivative thereof can be purified according to standard procedures of the art, including ammonium sulfate precipitation, affinity purification columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to about 95% homogeneity are preferred, and about 98 to about 99% or more homogeneity are most preferred. Once purified, partially or to homogeneity as desired, the polypeptides may then be used (e.g., as immunogens for antibody production).

To facilitate purification of the engineered reverse transcriptase or a derivative thereof, the nucleic acids that encode the engineered reverse transcriptase or derivatives thereof can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art as described herein, and several are commercially available (e.g., FLAG″ (Kodak, Rochester N.Y.). Another example of a suitable tag is a polyhistidine sequence, which is capable of binding to metal chelate affinity ligands. Typically, six adjacent histidines are used (6His-tag, his-tag), although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill in the art would recognize that after biological expression or purification, the engineered reverse transcriptase or derivatives thereof may possess a conformation substantially different than the native conformations of the constituent polypeptides. In this case, it may be necessary or desirable to denature and reduce the engineered reverse transcriptase or a derivative thereof and cause the engineered reverse transcriptase or a derivative thereof to re-fold into the preferred conformation. Methods of reducing and denaturing proteins and inducing re-folding are well known to those of skill in the art (See Debinski et al. (1993) J. Biol. Chem., 268: 14065-14070; Kreitman and Pastan (1993) Bioconjug. Chem., 4: 581-585; and Buchner et al. (1992) Anal. Biochem., 205: 263-270). Debinski et al., for example, describe the denaturation and reduction of inclusion body proteins in guanidine-DTE. The protein is then refolded in a redox buffer containing oxidized glutathione and L-arginine.

### IV. Compositions and Reaction Mixtures

The present disclosure further provides compositions comprising a variety of components in various combinations needed for nucleic acid amplification. In some embodiments of the present disclosure, the compositions are formulated by admixing one or more engineered reverse transcriptase enzymes or derivatives thereof of the present disclosure in a buffered salt solution. One or more DNA polymerases and/or one or more nucleotides, and/or one or more primers may optionally be added to create the compositions of the invention. These compositions can be used in the methods disclosed herein to produce, analyze, quantitate and otherwise manipulate nucleic acid molecules (e.g., using reverse transcription or one-step RT-PCR procedures).

In some embodiments, the engineered reverse transcriptase disclosed herein are provided at working concentrations (e.g., 1×) in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4º C, about two to six months at a temperature of about −20° C., and about six months or longer at a temperature of about −80° C. As used herein, the term “working concentration” means the concentration of an enzyme that is at or near the optimal concentration used in a solution to perform a particular function such as reverse transcription of nucleic acids.

Such compositions can also be formulated as concentrated stock solutions (e.g., 2×, 3×, 4×, 5×, 6×, 10×, etc.). In some embodiments, having the composition as a concentrated (e.g., 5×) stock solution allows a greater amount of nucleic acid sample to be added (such as, for example, when the compositions are used for nucleic acid synthesis). The water used in forming the compositions of the present invention is preferably distilled, deionized and sterile filtered (through a 0.1-0.2 micrometer filter), and is free of contamination by DNase and RNase enzymes. Such water is available commercially, for example from Life Technologies (Carlsbad, Calif.) or may be made as needed according to methods well known to those skilled in the art.

### V. Methods for Using Engineered Reverse Transcriptases

### A. Amplification Methods

One aspect of the present disclosure provides a method of using the engineered reverse transcriptase described herein, the method comprising contacting the engineered reverse transcriptase with a nucleic acid template under suitable conditions to produce a polymerized nucleic acid product. In some embodiments, the nucleic acid template is an RNA, or a nucleic acid comprising an unnatural nucleotide. The engineered reverse transcriptases of the present disclosure may be used in any application in which a reverse transcriptase with the indicated altered activity is desired. Methods of using reverse transcriptases are known in the art; one skilled in the art may select any of the engineered reverse transcriptases disclosed herein.

The engineered e reverse transcriptase enzyme or a derivative thereof as described herein may be used to make nucleic acid molecules from one or more templates. Such methods can comprise mixing one or more nucleic acid templates (e.g., RNA, such as non-coding RNA (ncRNA), messenger RNA (mRNA), micro RNA (miRNA), and small interfering RNA (siRNA) molecules) with one or more of the reverse transcriptases of the disclosure and incubating the mixture under conditions sufficient to generate one or more nucleic acid molecules complementary to all or a portion of the one or more nucleic acid templates. Other methods of cDNA synthesis which may advantageously use the present disclosure will be readily apparent to one of ordinary skill in the art.

In some embodiments, the method of using the engineered reverse transcriptase enzyme or a derivative thereof as described herein comprises the amplification of one or more nucleic acid molecules comprising mixing one or more nucleic acid templates with one of the engineered reverse transcriptase enzymes or a derivative thereof of the disclosure, and incubating the mixture under conditions sufficient to amplify one or more nucleic acid molecules complementary to all or a portion of the one or more nucleic acid templates. In one embodiment, the method may further comprise the use of one or more DNA polymerases and may be employed as in standard reverse transcription-polymerase chain reaction (RT-PCR) reactions.

In some embodiments, the method of using the engineered reverse transcriptase enzyme or a derivative thereof as described herein may be one-step (e.g., one-step RT-PCR) or two-step (e.g., two-step RT-PCR) reactions. In one embodiment, the one-step RT-PCR type reactions may be accomplished in one tube thereby lowering the possibility of contamination. Such one-step reactions comprise (a) mixing a nucleic acid template (e.g., mRNA) with one or more engineered reverse transcriptase enzymes or derivatives thereof of the present disclosure and one or more polymerases and (b) incubating the mixture under conditions sufficient to amplify a nucleic acid molecule complementary to all or a portion of the template.

In another embodiment, a two-step RT-PCR reaction may be accomplished in two separate steps. Such a method comprises (a) mixing a nucleic acid template (e.g., mRNA) with a engineered reverse transcriptase enzyme or a derivative thereof of the present disclosure, (b) incubating the mixture under conditions sufficient to make a nucleic acid molecule (e.g., a DNA molecule) complementary to all or a portion of the template, (c) mixing the nucleic acid molecule with one or more DNA polymerases and (d) incubating the mixture of step (c) under conditions sufficient to amplify the nucleic acid molecule. For amplification of long nucleic acid molecules (i.e., greater than about 3-5 kb in length), a combination of DNA polymerases and the engineered reverse transcriptase enzyme or a derivative thereof of the present disclosure may be used.

Amplification methods which may be used in accordance with the present invention (using one or more engineered reverse transcriptase enzymes or derivatives thereof of the present disclosure) include PCR, Isothermal Amplification, Strand Displacement Amplification (SDA), and Nucleic Acid Sequence-Based Amplification (NASBA); as well as more complex PCR-based nucleic acid fingerprinting techniques such as Random Amplified Polymorphic DNA (RAPD) analysis, Arbitrarily Primed PCR (AP-PCR) DNA Amplification Fingerprinting (DAF); microsatellite PCR; Directed Amplification of Minisatellite-region DNA (DAVID); digital droplet PCT (ddPCR) and Amplification Fragment Length Polymorphism (AFLP) analysis. See, e.g., EP 0 534 858; Vos, P., et al. Nucl. Acids Res. 23(21): 4407-4414 (1995); Lin, J. J., and Kuo, J. FOCUS 17(2):66-70 (1995); U.S. Pat. Nos. 4,683,195 and 4,683,202; PCT Publication No. WO 2006/081222; U.S. Pat. No. 5,455,166; EP 0 684 315. U.S. Pat. No. 5,409,818; EP 0 329 822; Williams, J. G. K., et al., Nucl. Acids Res. 18(22):6531-6535, (1990); Welsh, J., and McClelland, M., Nucl. Acids Res. 18(24): 7213-7218 (1990); Caetano-Anollés et al., Bio/Technology 9:553-557 (1991); Heath, D. D., et al. Nucl. Acids Res. 21(24): 5782-5785 (1993). Nucleic acid sequencing techniques which may employ the present compositions include dideoxy sequencing methods such as those disclosed in U.S. Pat. Nos. 4,962,022 and 5,498,523. In some embodiments, the engineered reverse transcriptase disclosed herein may be used in methods of amplifying or sequencing a nucleic acid molecule comprising one or more polymerase chain reactions (PCRs), such as any of the PCR-based methods described above.

Methods of producing an engineered reverse transcriptase or a derivative thereof of the present disclosure are known to those of skill in the art of molecular biology or molecular genetics. For example, nucleic acids encoding the wild type polymerase or nucleic acid binding domains can be generated using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); Current Protocols in Molecular Biology (Ausubel et al., eds., 1994-1999); Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

### B. Nucleic Acid Sample Processing

One aspect of the present disclosure provides a nucleic acid extension method comprising: contacting a target nucleic acid molecule with an engineered reverse transcriptase and a plurality of nucleic acid barcoded molecules comprising a barcode sequence, and incubating the target nucleic acid, the engineered reverse transcriptase and barcoded molecules under conditions in which the barcoded molecules are extended by the engineered reverse transcriptase. In some embodiments, the engineered reverse transcriptase comprises the amino acid sequence of an engineered transcriptase described herein or a derivatives thereof. The target nucleic acid hybridizes to one of the plurality of barcoded molecules and the hybridized barcoded molecule is extended by the engineered reverse transcriptase described herein.

### 1. RNA Template

In some embodiments, the nucleic acid is a ribonucleic acid (RNA) molecule; and the engineered reverse transcriptase enzyme reverse transcribes the RNA molecule thereby generating a first strand cDNA. In some embodiments, a reverse transcription reaction introduces a bar code. For example, in some embodiments a barcode is introduced during a reverse transcription amplification reaction that generates complementary deoxyribonucleic acid (cDNA) molecules upon reverse transcription of ribonucleic acid (RNA) molecules of the cell. In some embodiments, the RNA molecules are released from the cell. In some embodiments, the RNA molecules are released from the cell by permeabilizing or lysing the cell. In some embodiments, the RNA molecules are messenger RNA (mRNA).

In some embodiments, a reverse transcription reaction of the engineered reverse transcriptase enzyme of the present disclosure is initiated at the point of hybridization of the capture sequences to the RNA molecules, with the capture probe being extended by the engineered reverse transcriptase enzyme of the present disclosure in a template directed fashion using the hybridized mRNA as a template. In some embodiments, the reverse transcription reaction produces single stranded cDNA molecules each having a molecular tag and barcode associated with the cDNA, followed by amplification of cDNA to produce a double stranded cDNA that includes the sequences of the barcoded molecules.

In some embodiments, the plurality of nucleic acid barcoded molecules comprise an oligo(dT) sequence. In that embodiment, the engineered reverse transcriptase enzyme reverse transcribes the mRNA molecule into a complementary DNA molecule using the mRNA hybridized to the oligo(dT) sequence of the nucleic acid barcoded molecules as a template, and the nucleic acid binding domain binds and stabilizes the mRNA-oligo(dT) hybrid during the reverse transcription. Following reverse transcription, the engineered reverse transcriptase enzyme as described herein further amplifies the complementary DNA molecule comprising the barcode sequence, thereby generating an amplified DNA product comprising the barcode sequence, molecular tag sequence, or complements thereof.

In some embodiments of the nucleic acid extension method described herein, the method further comprises a second nucleic acid molecule comprising an oligo(dT) sequence. In that embodiment, the plurality of nucleic acid barcoded molecules further comprise an oligo(dT) sequence; and the nucleic acid binding domain of the engineered reverse transcriptase enzyme binds and stabilizes the mRNA-Oligo(dT) hybrid, while the polymerase domain of the engineered reverse transcriptase enzyme reverse transcribes the mRNA molecule using the second nucleic acid molecule comprising the oligo(dT) sequence, thereby generating a complementary DNA molecule. In this embodiment, the engineered reverse transcriptase enzyme further amplifies the complementary DNA molecule, thereby generating an amplified DNA product comprising a barcode sequence.

In some embodiments, the nucleic acid extension method further comprises a cell, a population of cells, or a tissue and the template nucleic acid molecule is from the cell, population of cells or the tissue.

In some embodiments, barcodes are coupled to primer sequences and the barcoding reaction is initiated by hybridization of the primer sequences to the RNA molecules. In some embodiments, each primer sequence comprises a random N-mer sequence. In some embodiments, the random N-mer sequence is complementary to a 3′ sequence of a ribonucleic acid molecule in said cell. In some embodiments, the random N-mer sequence of the primer sequence comprises a poly-dT sequence having a length of at least 5 bases. In some embodiments, the random N-mer sequence comprises a poly-dT sequence having a length of at least 10 bases (SEQ ID NO:17). In some embodiments, a barcode is introduced by extending the primer sequences in a template directed fashion using reagents for reverse transcription. In some embodiments, a molecular tag which comprises a barcode plus additional functional sequences, or only additional functional sequences, is further included into a cDNA molecule generated during a reverse transcription reaction. In some embodiments, the reagents for reverse transcription comprise a reverse transcription enzyme, a buffer and a mixture of nucleotides. In some embodiments, the reverse transcription enzyme adds a plurality of non-template oligonucleotides upon reverse transcription of a ribonucleic acid molecule from the nucleic acid molecules. In some embodiments, the reverse transcription enzyme is an engineered reverse transcription enzyme as disclosed herein.

In some embodiments, the barcoding reaction produces single stranded complementary deoxyribonucleic acid (cDNA) molecules each having a barcode on a 5′ end thereof, followed by amplification of cDNA to produce a double stranded cDNA having the barcode on the 5′ end and a molecular tag which may or may not include a barcode on a 3′ end of the double stranded cDNA.

In one aspect, the present invention provides methods that utilize the engineered reverse transcriptases described herein for nucleic acid sample processing. In one embodiment, the method comprises contacting a template ribonucleic acid (RNA) molecule with an engineered reverse transcriptase to reverse transcribe the RNA molecule to a complementary DNA (cDNA) molecule. The contacting step may be in the presence of a plurality of nucleic acid barcode molecules, wherein each nucleic acid barcode molecule comprises a barcode sequence. The nucleic acid barcode molecule may further comprise a sequence configured to couple to a template RNA molecule. Suitable sequences include, without limitation, an oligo(dT) sequence, a random N-mer primer, or a target-specific primer. The nucleic acid barcode molecule may further comprise a template switching sequence. In other embodiments, the RNA molecule is a messenger RNA (mRNA) molecule. In one embodiment, contacting step provides conditions suitable to allow the engineered reverse transcriptase to (i) transcribe the mRNA molecule into the cDNA molecule with the oligo(dT) sequence and/or (ii) perform a template switching reaction, thereby generating the cDNA molecule which comprises the barcode sequence, or a derivative thereof. In another embodiment, the contacting step may occur in (i) a partition having a reaction volume (as further described herein and see e.g., U.S. Pat. Nos. 10,400,280 and 10,323,278, each of which is incorporated herein by reference in its entirety), (ii) in a bulk reaction where the reaction components (e.g., template RNA and engineered reverse transcriptase) are in solution, or (iii) on a nucleic acid array (see e.g., U.S. Pat. No. 10,480,022 and 10,030,261 as well as WO/2020/047005 and WO/2020/047010, each of which is incorporated herein by reference in its entirety). Further, the reverse transcription reaction may occur in a tissue (in situ reverse transcription), on a template that is associated with a sequence on a substrate such as practiced in spatial transcriptomics, or further in a RT-PCR or other reverse transcription reaction in vitro on a purified target, partially purified target or unpurified target as found for example in a cellular lysate.

Examples of assays involving nucleic acid sample processing may include, but are not limited to, single-cell transcription profiling, single-cell sequence analysis, immune profiling of individual T and B cells, single-cell chromatin accessibility analysis (e.g. ATAC seq analysis), single cell processing and analysis, paired single cell TCR sequencing, paired TCRα and TCRβ. These exemplary assays may be carried out using commercially available systems for encapsulating biological samples, gel beads, barcodes, and/or other compounds/materials in droplets, such as The Chromium System (10X Genomics, Pleasanton CA USA). Engineered reverse transcriptases may be used in methods of profiling a T-Cell receptor (TCR) such as those described in U.S. Provisional Application No. 62/902,178, herein incorporated by reference in its entirety.

In various embodiments, the poly-dT sequence may be extended in a reverse transcription reaction using the mRNA as a template to produce a cDNA transcript complementary to the mRNA and also includes sequence of a barcode oligonucleotide. Terminal transferase activity of the reverse transcriptase can add additional bases to the cDNA transcript (e.g., polyC). The switch oligo may then hybridize with the additional bases added to the cDNA transcript and facilitate template switching. A sequence complementary to the switch oligo sequence can then be incorporated into the cDNA transcript via extension of the cDNA transcript using the switch oligo as a template. Within any given partition, all the cDNA transcripts of the individual mRNA molecules include a common barcode sequence. However, by including the unique random N-mer sequence, the transcripts made from different mRNA molecules within a given partition will vary at this unique sequence. As described elsewhere herein, this provides a quantification feature that can be identifiable even following any subsequent amplification of the contents of a given partition, e.g., the number of unique segments associated with a common barcode can be indicative of the quantity of mRNA originating from a single partition, and thus, a single cell. The cDNA transcript may then be amplified with PCR primers. The amplified product may then be purified (e.g., via solid phase reversible immobilization (SPRI)). The amplified product can be ligated to additional functional sequences, and further amplified (e.g., via PCR). The functional sequences may include a sequencer specific flow cell attachment sequence such as but not limited to, a P7 sequence for Illumina sequencing systems, as well as functional sequence, which may include a sequencing primer binding site, e.g., for a R2 primer for Illumina sequencing systems, as well as functional sequence, which may include a sample index, e.g., an i7 sample index sequence for Illumina sequencing systems.

Although described in terms of specific sequence references used for certain sequencing systems, e.g., Illumina systems, it will be understood that the reference to these sequences is for illustration purposes only, and the methods described herein may be configured for use with other sequencing systems incorporating specific priming, attachment, index, or other operational sequences used in those systems, e.g., systems available from Ion Torrent, Oxford Nanopore, Genia, Pacific Biosciences, Complete Genomics, and the like.

### 2. Volume

As described herein, wild-type and variants MMLV RT are not optimal for reverse transcription of mRNA when using high throughput amplification reaction assays (e.g. spatial array and single cell transcriptomics assay) and the like. This is because high throughput amplification reaction assays require reaction volumes that are usually less than about 1 nanoliter. Accordingly, the present disclosure provides novel engineered reverse transcriptase enzymes that function efficiently in high throughput amplification reaction assays that require reaction volumes of less than about 1 nanoliter.

In some embodiments, the method comprises providing a reaction volume which comprises an engineered reverse transcriptase and a template ribonucleic acid (RNA) molecule. In one other embodiment, the contacting occurs in a reaction volume, which may be less than 1 nanoliter, less than 750 picoliters, or less than 500 picoliters. In other embodiments, the reaction volume is present in a partition, such as a droplet or well (including a microwell or a nanowell).

In some embodiments, the engineered reverse transcriptase enzymes or derivatives thereof as described herein are used in a reaction volume less than about 1 nanoliter (nL). In some embodiments, the engineered reverse transcriptase enzymes or derivatives thereof as described herein are used in a reaction volume that is less than about 500 picoliter (pL). In some embodiments, the reaction volume is contained within a partition. In some embodiments, the reaction volume is contained within a droplet. In some embodiments, the reaction volume is contained within a droplet in an emulsion. In some embodiments, the reaction volume is contained within a droplet emulsion having a reaction volume of less than about 1 nL. In some embodiments, the reaction volume is contained within a droplet emulsion having a reaction volume of less than about 500 pL.

In some embodiments, the reaction volume is contained within a well. In some embodiments, the reaction volume is contained within a well having a reaction volume less than about 1 nL. In some embodiments, the reaction volume is contained within a well. In some embodiments, the reaction volume is contained within a well having a reaction volume less than about 500 pL. In some embodiments, the reaction volume is contained within a well in an array of wells having an extracted nucleic acid molecule, and the template nucleic acid molecule is the extracted nucleic acid molecule. In some embodiments, the reaction volume is contained within a well in an array of wells having a cell comprising a template nucleic acid molecule, and where the template nucleic acid molecule is released from the cell.

### 3. Unique Molecular Identifier (UMI)

In some embodiments, molecular tags, which may or may not include a barcode, further include a functional sequence such as a unique molecular identifier (UMI). In some embodiments, molecular tags are coupled to primer sequences. In some embodiments, each of said primer sequences comprises a random N-mer sequence. In some embodiments, the random N-mer sequence is complementary to a 3′ sequence of said RNA molecules. In some embodiments, the primer sequence comprises a poly-dT sequence having a length of at least 5 bases. In some embodiments, the primer sequence comprises a poly-dT sequence having a length of at least 10 bases (SEQ ID NO: 17). In some embodiments, the primer sequence comprises a poly-dT sequence having a length of at least 5 bases, at least 6 bases, at least 7 bases, at least 8 bases, at least 9 bases, at least 10 bases (SEQ ID NO: 17).

Unique molecular identifiers (UMIs) are assigned or associated with individual cells or populations of cells, in order to tag or label the cell's components (and as a result, its characteristics) with the unique identifiers. These unique molecular identifiers may be used to attribute the cell's components and characteristics to an individual cell or group of cells.

In some aspects, the unique molecular identifiers are provided in the form of nucleic acid molecules (e.g., oligonucleotides) that comprise nucleic acid barcode sequences that may be attached to or otherwise associated with the nucleic acid contents of individual cell, or to other components of the cell, and particularly to fragments of those nucleic acids. The nucleic acid molecules are partitioned such that as between nucleic acid molecules in a given partition, the nucleic acid UMI sequences contained therein are the same, but as between different partitions, the nucleic acid molecule can, and do have differing UMI sequences, or at least represent a large number of different UMI sequences across all of the partitions in a given analysis. In some aspects, only one nucleic acid barcode or UMI sequence can be associated with a given partition, although in some cases, two or more different barcode or UMI sequences may be present.

The nucleic acid UMI or barcode sequences can include from about 6 to about 20 or more nucleotides within the sequence of the nucleic acid molecules (e.g., oligonucleotides). The nucleic acid UMI or barcode sequences can include from about 6 to about 20, 30, 40, 50, 60, 70, 80, 90, 100 or more nucleotides. In some cases, the length of a UMI or barcode sequence may be about 6, 7, 8, 9, 10, 11, 12, 13, 14, 15, 16, 17, 18, 19, 20 nucleotides or longer. In some cases, the length of a UMI or barcode sequence may be at least about 6, 7, 8, 9, 10, 11, 12, 13, 14, 15, 16, 17, 18, 19, 20 nucleotides or longer. In some cases, the length of a UMI or barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated UMI or barcode subsequences can be from about 4 to about 16 nucleotides in length. In some cases, the UMI or barcode subsequence may be about 4, 5, 6, 7, 8, 9, 10, 11, 12, 13, 14, 15, 16 nucleotides or longer. In some cases, the UMI or barcode subsequence may be at least about 4, 5, 6, 7, 8, 9, 10, 11, 12, 13, 14, 15, 16 nucleotides or longer. In some cases, the UMI or barcode subsequence may be at most about 4, 5, 6, 7, 8, 9, 10, 11, 12, 13, 14, 15, 16 nucleotides or shorter.

Moreover, when a population of barcodes or UMIs is partitioned, the resulting population of partitions can also include a diverse barcode or UMI library that may include at least about 1,000 different barcode or UMI sequences, at least about 5,000 different barcode or UMI sequences, at least about 10,000 different barcode or UMI sequences, at least about 50,000 different barcode or UMI sequences, at least about 100,000 different barcode or UMI sequences, at least about 1,000,000 different barcode or UMI sequences, at least about 5,000,000 different barcode or UMI sequences, or at least about 10,000,000 different barcode or UMI sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some embodiments, the enhanced reverse transcriptase activity of the engineered reverse transcriptase disclosed herein is an enhanced ability to yield mitochondrial UMI counts as compared to a reverse transcriptase having the amino acid sequence set forth in SEQ ID NO:1 or 15. In some embodiments, the enhanced reverse transcriptase activity is an enhanced ability to yield increased ribosomal UMI counts as compared to a reverse transcriptase having the amino acid sequence set forth in SEQ ID NO: 1 or 15. Read counting and unique molecular identifier (UMI) counting are the principal gene expression quantification schemes used in single-cell RNA-sequencing (scRNA-seq) analysis, as such with increased ribosomal UMI counts sensitivity and accuracy increases for a scRNA-seq assay in determining transcriptome profiles for any given cell, group of cells or tissues. Numerous metrics can be used for quality control of single-cell RNA-sequencing, including percent of reads mapping to ribosomal genes, percent of reads mapping to mitochondrial genes, total number of UMIs detected, or number of features to which 50% of the reads map.

Beneficially, even following any subsequent amplification of the contents of a given partition, the number of different UMIs can be indicative of the quantity of mRNA originating from a given partition, and thus from the cell. As noted above, the transcripts can be amplified, purified and sequenced to identify the sequence of the cDNA transcript of the mRNA, as well as to sequence the barcode segment and the UMI segment. While a poly-dT primer sequence is described, other targeted or random primer sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

It is recognized that certain reverse transcriptase enzymes may increase UMI reads from genes of a desired length or length of interest. The desired length of genes may be selected from the group of lengths comprising less than 500 nucleotides, between 500 and 1000 nucleotides, between 1000 and 1500 nucleotides and greater than 1500 nucleotides. It is recognized that a reverse transcriptase may preferentially increase UMI reads from genes of one length range. It is recognized that an engineered reverse transcriptase may perform similarly, differently or comparably in a 3′-reverse transcription assay or a 5′-reverse transcription assay. It is similarly recognized that an engineered reverse transcriptase may preferentially increase UMI reads from a length of genes in a 3′-reverse transcription assay than in a 5′-reverse transcription assay.

### 4. Gel Bead

The engineered reverse transcriptases of the present application may be suitable for use in methods in which a cell can be co-partitioned along with a barcode and/or UMI bearing bead. The barcoded nucleic acid molecules can be released from the bead in the partition. By way of example, in the context of analyzing sample RNA, the poly-dT (poly-deoxythymine, also referred to as oligo (dT)) segment of one of the released nucleic acid molecules can hybridize to the poly-A tail of a mRNA molecule. Reverse transcription may result in a cDNA transcript of the mRNA, but which transcript includes each of the sequence segments of the nucleic acid molecule. Without being limited by mechanism, because the nucleic acid molecule comprises an anchoring sequence, it may be more likely to hybridize to and prime reverse transcription at the sequence end of the poly-A tail of the mRNA. Within any given partition, substantially all of the cDNA transcripts of the individual mRNA molecules may include a common barcode sequence segment. However, the transcripts made from the different mRNA molecules within a given partition may vary at the unique molecular identifying sequence segment (e.g., UMI segment).

In some embodiments of the nucleic acid extension method described herein, the plurality of nucleic acid barcoded molecules are attached to a support (e.g. a particle, a slide, a chip, a bead, etc.). In one embodiment, the support is selected from the group consisting of an array, a bead, a gel bead, a microparticle, and a polymer. In some embodiments, the nucleic acid barcoded molecules attached to a support comprise molecular tags (UMIs), primer sequences, capture sequences, cleavage sequences, or additional functional sequences. In some embodiments, the support is a gel bead. In that embodiment, the nucleic acid barcoded molecules are releasably attached to the gel bead. In some embodiments, the gel bead comprises a polyacrylamide polymer.

In some embodiments, a cross-section of the gel bead is less than about 100 μm. In some embodiments, a cross-section of a gel bead is less than about 60 μm. In some embodiments, a cross-section of a gel bead is less than about 50 μm. In some embodiments, a cross-section of a gel bead is less than about 40 μm. In some embodiments, a cross-section of a gel bead is less than about 100 μm, less than about 99 μm, less than about 98 μm, less than about 97 μm, less than about 96 μm, less than about 95 μm, less than about 94 μm, less than about 93 μm, less than about 92 μm, less than about 91 μm, less than about 90 μm, less than about 89 μm, less than about 88 μm, less than about 87 μm, less than about 86 μm, less than about 85 μm, less than about 84 μm, less than about 83 μm, less than about 82 μm, less than about 81 μm, less than about 80 μm, less than about 79 μm, less than about 78 μm, less than about 77 μm, less than about 76 μm, less than about 75 μm, less than about 74 μm, less than about 73 μm, less than about 72 μm, less than about 71 μm, less than about 70 μm, less than about 69 μm, less than about 68 μm, less than about 67 μm, less than about 66 μm, less than about 65 μm, less than about 64 μm, less than about 63 μm, less than about 62 μm, less than about 61 μm, or less than about 60 μm.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 sequence for use in Illumina sequencing workflows. In some cases, the primer can comprise an R2 sequence for use in Illumina sequencing workflows. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference. However, the present invention is not limited as to a composition of any nucleic acid molecule or derivative thereof, or any particular sequencing platform and these characterizations serve as examples only which may be useful in a reverse transcription workflow.

In operation, a cell can be co-partitioned along with a barcode bearing bead. The barcoded nucleic acid molecules affixed to a bead can be released from the bead in the partition. By way of example, in the context of analyzing sample RNA, the poly-dT (poly-deoxythymine, also referred to as oligo (dT)) segment of one of the released nucleic acid molecules can hybridize to (e.g., capture)_the poly-A tail of a mRNA molecule. Reverse transcription may result in a cDNA transcript of the mRNA which cDNA transcript also includes each of the sequence segments of the nucleic acid molecule. Because the nucleic acid molecule comprises additional functional sequences (e.g., capture domains, primer domains, UMIs, barcodes, etc.), it can hybridize to and prime reverse transcription of the mRNA using the hybridized mRNA as a template. Within any given partition, all of the cDNA transcripts of the individual mRNA molecules may include a common barcode sequence. However, the transcripts made from the different mRNA molecules within a given partition may vary with respect to unique molecular identifying sequences (e.g., UMIs). Beneficially, following any subsequent amplification of the contents of a given partition, the number of different UMIs can be indicative of the quantity of mRNA originating from a given partition, and thus from the cell. As noted above, the transcripts can be amplified and sequenced to identify the sequence of the original mRNA captured template, as well as the sequence of the associated barcode and UMI. While a poly-dT capture sequence is described, other targeted or random capture sequences may also be used in capture or hybridize to a template for initiating the reverse transcription reaction.

### C. Processing TCR

In some embodiments, an engineered reverse transcriptase is used in methods including but not limited to processing of a TCR from an individual T cell(s) or groups of T cell(s), determining the nucleotide sequence of the TCR(s) of T cell(s), and obtaining TCR repertoire profile. In some methods, a nucleic acid barcode sequence is appended to a nucleic acid molecule encoding for a TCR (e.g. a molecule derived from a T cell containing a nucleic acid sequence encoding for a TCR, such as a TCRa and/or a TCRb mRNA) resulting in a barcoded nucleic acid molecule comprising a sequence corresponding to a nucleic acid sequence of the TCR (e.g. comprises a V(D)J region of a TCR gene or a reverse complement thereof) and a sequence corresponding to the barcode sequence (which in some instances is the reverse complement of the barcode sequence present in the nucleic acid barcode molecule). A barcoded nucleic acid molecule may serve as a template, such as a template polynucleotide, that can be further processed (e.g. amplified) and sequenced to obtain the target nucleic acid sequence. For example, a barcoded nucleic acid molecule may be further processed (e.g. amplified) and sequenced to obtain the nucleic acid sequence of the TCR.

TCR is a molecule found on the surface of T cells. Typically binding of the TCR by an antigenic molecule results in cell activation and response. The TCR is a heterodimer composed of two different protein chains. In many T cells, these two proteins are alpha (a) and beta (B) chains. In a smaller percentage of T cells, these two proteins are gamma (γ) and delta (8) chains. The ratio of TCRs comprised of a/ß chains versus γ/δ chains may change during a diseased state such as cancer, tumor, infectious disease, inflammatory disease or autoimmune disease. Engagement of the TCR with a peptide-MHC activates a T cell through a series of biochemical events mediated by associated enzymes, co-receptors, specialized adaptor molecules, and activated or released transcription factors.

Each of the two chains of a TCR contains multiple copies of gene segments—a variable ‘V’ gene segment, a diversity ‘D’ segment and a joining ‘J’ segment. The TCR alpha chain is generated by recombination of V and J segments, while the beta chain is generated by recombination of V, D and J segments. Similarly, generation of the TCR gamma chain involves recombination of V and J segments. Generation of the TCR delta chain occurs by recombination of V, D and J gene segments. The intersection of these specific regions (V and J for the alpha or gamma chain, or V, D, J for the beta or delta chain) corresponds to the CDR3 region involved in antigen-MHC recognition. Complementarity determining regions (e.g. CDR1, CDR2 and CDR3) or hypervariable regions are sequences in the variable domains of antigen receptors (e.g. T cell receptor and immunoglobulin) that can complement an antigen. Most of the diversity of CDRs is found in CDR3, with the diversity being generated by somatic recombination events during the development of T lymphocytes. CDR3, which is encoded by the junctional region between the V and J or D and J genes, is highly variable. CDR3 is often used as a region of interest to determine T cell clonotypes, a unique nucleotide sequence that arises during the gene rearrangement process, as it is highly unlikely that two T cells will express the same CDR3 nucleotide sequence unless they are derived from the same clonally expanded T cell. Because an active TCR consists of paired chains within single T cells, determination of the active paired chains within single T cells, determination of the active paired chains requires the sequencing of single T cells. TCR gene sequences may include, but are not limited to, sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes) and T cell receptor delta constant genes (TRDC genes).

### VII. Kits

One aspect of the present invention provides a kit comprising the engineered reverse transcriptase enzyme or a derivative thereof as described herein. In some embodiments, the kit further comprises one or more of a vector, a nucleotide, a buffer, a salt, and/or instructions. In another embodiment, a kit may comprise an engineered reverse transcriptase enzyme or a derivative thereof for use in reverse transcription or amplification of a nucleic acid molecule. In yet another embodiment, a kit may be used for single cell profiling of the transcriptome. In yet another embodiment, a kit may be used for spatial transcriptomics methods and assays. In yet another embodiment, a kit may be used for in situ methods and assays.

The kit may include suitable reaction buffers, dNTPs, one or more primers, one or more control reagents, or any other reagents disclosed for performing the methods of the present disclosure. The engineered reverse transcriptase enzyme or a derivative thereof, reaction buffer, and dNTPs may be provided separately or may be provided together in a master mix solution. When the engineered reverse transcriptase enzyme or a derivative thereof, reaction buffer, and dNTPs are provided in a master mix, the master mix is present at a concentration at least two times the working concentration indicated in instructions for use in an extension reaction. In other cases, the master mix may be present at a concentration at least three times, at least four times, at least five times, at least six times, at least seven times, at least eight times, at least nine times, or at least ten times, the working concentration indicated. The primer in the kits may be a poly-dT primer, a random N-mer primer, or a target-specific primer.

The kits may further include one, two, three, four, five or more, up to all of partitioning fluids, including both aqueous buffers and non-aqueous partitioning fluids or oils, nucleic acid barcode capture probes that are releasably associated with beads, as described herein, microfluidic devices, reagents for disrupting cells, reagents for amplifying nucleic acids, as well as instructions for using any of the foregoing in the methods described herein.

The instructions for using any of the methods are generally recorded on a suitable recording medium (e.g. printed on a substrate such as paper or plastic), or available in a digital format. As such, the instructions may be present in the kits as a package insert, in the labeling of the container of the kit or components thereof (i.e., associated with the packaging or subpackaging). In some cases, the instructions may be present as an electronic storage data file present on a suitable computer readable storage medium. In other cases, the actual instructions may not be present in the kit, but means for obtaining the instructions from a remote source, e.g. via the internet, may be provided. For example, a kit that includes a web address where the instructions may be viewed and/or from which the instructions may be downloaded. As with the instructions, this means for obtaining the instructions is recorded on a suitable substrate.

Kits according to this aspect of the present disclosure comprise a carrier means, such as a box, carton, tube or the like, having in close confinement therein one or more container means, such as vials, tubes, ampoules, bottles and the like, wherein a first container means contains one or more of the engineered reverse transcriptase enzymes or derivatives thereof of the present disclosure having reverse transcriptase activity. When more than one polypeptide having reverse transcriptase activity is used, they may be in a single container as mixtures of two or more engineered reverse transcriptase enzymes or derivatives thereof, or in separate containers. The kits of the disclosure can also comprise (in the same or separate containers) one or more DNA polymerases, a suitable buffer, one or more nucleotides and/or one or more primers.

The kits of the disclosure can also comprise one or more hosts or cells including those that are competent to take up nucleic acids (e.g., DNA molecules including vectors). Preferred hosts may include chemically competent or electrocompetent bacteria such as E. coli (including DH5, DH5a, DH10B, HB101, Top 10, and other K-12 strains as well as E. coli B and E. coli W strains).

In a specific aspect of the present disclosure, the kits of the disclosure (e.g., reverse transcription and amplification kits) can include one or more components (in mixtures or separately) including one or more engineered reverse transcriptase enzymes or derivative thereof having reverse transcriptase activity of the disclosure, one or more nucleotides (one or more of which may be labeled, e.g., fluorescently labeled) used for synthesis of a nucleic acid molecule, and/or one or more primers (e.g., oligo(dT) for reverse transcription, randomers for extension reactions, etc). Such kits can further comprise one or more DNA polymerases.

### VIII. Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. “A and/or B” is used herein to include all of the following alternatives: “A”, “B”, “A or B”, and “A and B”. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to +5%, or up to +1%. Numeric ranges are inclusive of the numbers defining the range. The term about is used herein to mean plus or minus ten percent (10%) of a value. For example, “about 100” refers to any number between 90 and 110.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, the term “analyte” is intended a biological molecule. Analytes include but are not limited to a DNA analyte, an RNA analyte, an oligonucleotide, a reporter molecule, a reporter molecule configured to directly couple to a protein, a reporter molecule configured to indirectly couple to a protein, a reporter molecule configured to directly couple to a metabolite, and a reporter molecule configured to indirectly couple to a metabolite.

The terms “Adaptor(s),” “Adapter(s)” and “Tag(s)” may be used synonymously. An adaptor or tag can be coupled to a polynucleotide sequence to be “tagged” by any approach, including ligation, hybridization, or other approaches.

As used herein, the term “barcoded nucleic acid molecule” generally refers to a nucleic acid molecule that results from, for example, the processing of a nucleic acid barcoded molecule with a nucleic acid sequence (e.g., nucleic acid sequence complementary to a nucleic acid primer sequence encompassed by the nucleic acid barcoded molecule). The nucleic acid sequence may be a targeted sequence or a non-targeted sequence. The nucleic acid barcoded molecule may be coupled to or attached to the nucleic acid molecule comprising the nucleic acid sequence. For example, a nucleic acid barcoded molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcoded molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcoded molecule to generate the barcoded nucleic acid molecule, or the nucleic acid molecule comprising the nucleic acid sequence may be attached to the nucleic acid barcoded molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

A nucleic acid barcoded molecule of a plurality of nucleic acid molecules may be used to generate a “barcoded nucleic acid molecule.” In some cases, a barcoded molecule comprises a different reporter barcode sequence that identifies a second analyte. A different reporter barcode sequence or an analyte-specific barcode sequence may identify a protein, a lipid, a metabolite or other second analyte.

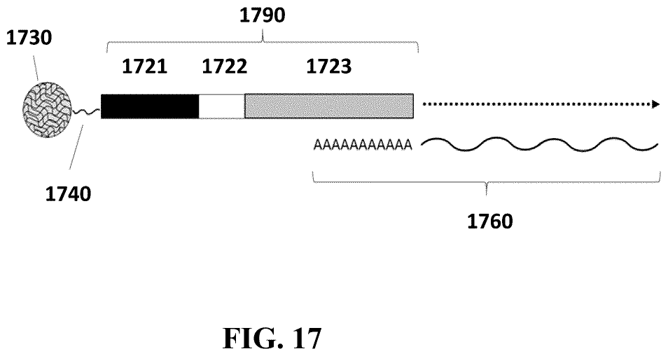

Barcoded nucleic acids may be generated (e.g., via a nucleic acid reaction, such as nucleic acid extension or ligation) from the constructs described in FIG. 17. For example, capture handle sequence may then be hybridized to complementary sequence, such as capture sequence 1723 to generate (e.g., via a nucleic acid reaction, such as nucleic acid extension or ligation) a barcoded nucleic acid molecule comprising cell (e.g., partition specific) barcode sequence 1722 (or a reverse complement thereof) and reporter barcode sequence 1722 (or a reverse complement thereof). In some embodiments capture handle sequence 1723 comprises a sequence complementary to a template switching oligonucleotide on the capture sequence 1723. In some embodiments, the nucleic acid barcoded molecule 1790 (e.g., partition-specific barcoded molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. In some instances, analysis of an analyte (e.g. a nucleic acid, a polypeptide, a carbohydrate, a lipid, a glycan, a glycan motif, a metabolite, a protein, etc.) comprises a workflow as generally depicted in FIG. 17. A nucleic acid barcoded molecule 1790 (e.g. partition specific barcoded molecule) may be co-partitioned with the one or more analytes. In some instances, nucleic acid barcoded molecule 1790 is attached to a support 1730 (e.g., a bead, such as a gel bead), such as those described elsewhere herein. For example, nucleic acid barcoded molecule 1790 may be attached to support 1730 via a releasable linkage 1740 (e.g., comprising a labile bond), such as those described elsewhere herein. Nucleic acid barcoded molecule 1790 may comprise a functional sequence 1721 and optionally comprise other additional sequences, for example, a barcode sequence 1722 (e.g., common barcode, partition-specific barcode, or other functional sequences described elsewhere herein), and/or a UMI sequence (not shown). The nucleic acid barcoded molecule 1790 may comprise a capture sequence 1723 that may be complementary to another nucleic acid sequence, such that it may hybridize to a particular sequence, e.g., capture handle sequence 1723.

For example, capture sequence 1723 may comprise a poly-T sequence and may be used to hybridize to mRNA. Referring to FIG. 17, in some embodiments, nucleic acid barcoded molecule 1790 comprises capture sequence 1723 complementary to a sequence of RNA molecule 1760 from a cell. In some instances, capture sequence 1723 comprises a sequence specific for an RNA molecule. Capture sequence 1723 may comprise a known or targeted sequence or a random sequence. In some instances, a nucleic acid extension reaction may be performed, thereby generating a barcoded nucleic acid product comprising capture sequence 1723, the functional sequence 1721, barcode sequence 1722, any other functional sequence, and a sequence corresponding to the RNA molecule 1760.

In another example, capture sequence 1723 may be complementary to an overhang sequence or an adapter sequence that has been appended to an analyte. Any suitable agent may degrade beads. Suitable agents may include, but are not limited to, changes in temperature, changes in pH, reduction, oxidation and exposure to water or other aqueous solutions.

In some instances, a cell that is bound to labelling agent which is conjugated to oligonucleotide and support 1730 (e.g., a bead, such as a gel bead) comprising nucleic acid barcoded molecule 1790 is partitioned into a partition amongst a plurality of partitions (e.g., a droplet of a droplet emulsion or a well of a microwell array).

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “efficiency” in the context of a nucleic acid modifying enzyme of this invention refers to the ability of the enzyme to perform its catalytic function under specific reaction conditions. Typically, “efficiency” as defined herein is indicated by the amount of product generated under given reaction conditions.

As used herein, the term “enhances” in the context of an enzyme refers to improving the activity of the enzyme, i.e., increasing the amount of product per unit enzyme per unit time.

As used herein, the term “fidelity” refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

As used herein, the term “% homology,” which is used interchangeably with the term “% identity,” refers to the level of nucleic acid or amino acid sequence identity between the nucleic acid sequence that encodes any one of the inventive polypeptides or the inventive polypeptide's amino acid sequence, when aligned using a sequence alignment program.

As used herein, the term “identical” in the context of two nucleic acids or polypeptide sequences refers to the residues in the two sequences that are the same when aligned for maximum correspondence, as measured using a sequence comparison algorithms. Sequence comparison algorithms are know to those skill in the art. See. E.g., ebi.ac.uk/Tools/msa/clustalo/.

As used herein, the term “inhibitor resistance” refers to the ability of a reverse transcriptase to perform reverse transcription in the presence of a compound, chemical, protein, buffer, etc. that is typically inhibitory to the reverse transcriptase (prevents or inhibits reverse transcriptase activity).

As used herein, the term “Low volume reaction” means a reaction volume less than 1 nanoliter, less than 750 picoliters, or less than 500 picoliters.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

As used herein, the term “mutation” or “mutant” or “variant” indicates a change or changes introduced in a wild type DNA sequence or a wild type amino acid sequence. Examples of mutations or variants include, but are not limited to, substitutions, insertions, deletions, and point mutations. Mutations can be made either at the nucleic acid level or at the amino acid level.

As used herein, the term “operably linked” or “conjugated” or “fusion” means that, in relation to the recombinant thermostable polymerase enzyme sequence there are one or more sequences at the N or C terminus that, when transcribed and translated, create additional polypeptides in association with the enzyme amino acid sequence, thereby created a conjugation or fusion of one or more polypeptides from one expression vector.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “partitioning” as used herein is intended to encompass parting, dividing, depositing, separating, or compartmentalizing into one or more partitions. Systems and methods for partitioning of one or more particles (such as, but not limited to, biological particles, macromolecular constituents of biological particles, beads, reagents, etc.) into discrete compartments or partitions (referred to interchangeably here as partitions), wherein each partition maintains separation of its own content from the contents of other partitions are known in the art. See for example US 2020/0032335, herein incorporated by reference in its entirety. The partition can be a droplet in an emulsion. A partition may comprise one or more other partitions.

A “plurality of nucleic acid barcoded molecules” may comprise at least about 500 nucleic acid barcoded molecules, at least about 1,000 nucleic acid barcoded molecules, at least about 5,000 nucleic acid barcoded molecules, at least about 10,000 nucleic acid barcoded molecules, at least about 50,000 nucleic acid barcoded molecules, at least about 100,000 nucleic acid barcoded molecules, at least about 500,000 nucleic acid barcoded molecules, at least about 1,000,000 barcoded molecules, at least about 5,000,000 nucleic acid barcoded molecules, at least about 10,000,000 nucleic acid barcoded molecules, at least about 100,000,000 nucleic acid barcoded molecules, at least about 1,000,000,000 nucleic acid barcoded molecules. In some cases, a plurality of nucleic acid barcoded molecules comprise a partition-specific barcode sequence.

Each of the plurality of nucleic acid barcoded molecules may include an identifier sequence separate from the partition-specific barcode sequence, where the identifier sequence is different for each nucleic acid partition-specific barcoded molecule of the plurality of nucleic acid partition specific barcoded molecules. In some cases, such an identifier sequence is a unique molecular identifier (UMI) as described elsewhere herein. As described elsewhere herein, UMI sequences can uniquely identify a particular nucleic acid molecule that is barcoded, which may be identifying particular nucleic acid molecules that are analyzed, counting particular nucleic acid molecules that are analyzed, etc. Furthermore, in some cases, each of the plurality of nucleic acid barcoded molecules can comprise the partition specific barcode sequence and the bead can be from plurality of beads, such as a population of barcoded beads. Each of the partition specific barcode sequences can be different from partition specific barcode sequences of nucleic acid barcoded molecules of other beads of the plurality of beads. Where this is the case, a population of barcoded beads, with each bead comprising a different partition specific barcode sequence can be analyzed.

As used herein, the term “processivity” refers to the ability of a reverse transcriptase to continuously extend a primer without disassociating from the nucleic acid template. The length of a template a reverse transcriptase or polymerase is capable of replicating can also be used to describe the processivity of that reverse transcriptase or polymerase. In some embodiments, “Processivity” refers to the ability of a polymerase to remain bound to the template or substrate and perform DNA synthesis. Processivity is measured by the number of catalytic events that take place per binding event.

As used herein, the term “Purified” means that a molecule is present in a sample at a concentration of at least 95% by weight, or at least 98% by weight of the sample in which it is contained.

As used herein, the term “reverse transcriptase activity,” “reverse transcription activity,” or “reverse transcription” indicates the capability of an enzyme to synthesize a DNA strand (that is, complementary DNA or cDNA) using RNA as a template. Reverse transcriptase activity may be measured by incubating an enzyme in the presence of an RNA template and deoxynucleotides, in the presence of an appropriate buffer, under appropriate conditions, for example as described in the Example below. Methods for measuring RT activity are provided in the example below and also are well known in the art. Bosworth, et al., Nature 1989, 341:167-168.

As used herein, the term “Reverse transcriptase (RT)” is used in its broadest sense to refer to any enzyme that exhibits reverse transcription activity as measured by methods disclosed herein or known in the art. A “reverse transcriptase” of the present invention, therefore, includes reverse transcriptases from retroviruses, other viruses, as well as a DNA polymerase exhibiting reverse transcriptase activity, such as Tth DNA polymerase, Taq DNA polymerase, Tne DNA polymerase, Tma DNA polymerase, etc. RT from retroviruses include, but are not limited to, Moloney Murine Leukemia Virus (M-MLV) RT, Human Immunodeficiency Virus (HIV) RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus UR2AV RT, Avian Sarcoma Virus Y73 Helper Virus YAV RT, Rous Associated Virus (RAV) RT, and Myeloblastosis Associated Virus (MAV) RT, and as described in U.S. Patent Application 2003/0198944 (hereby incorporated by reference in its entirety). For review, see e.g. Levin, 1997, Cell, 88:5-8; Brosius et al. 5 1995, Virus Genes 11:163-79. Known reverse transcriptases from viruses require a primer to synthesize a DNA transcript from an RNA template. Reverse transcriptase has been used primarily to transcribe RNA into cDNA, which can then be cloned into a vector for further manipulation or used in various amplification methods such as polymerase chain reaction (PCR), nucleic acid sequence-based amplification (NASBA), transcription mediated amplification (TMA), or self-sustained sequence replication (3SR).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “Sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term “thermoreactivity” or “thermoreactive” refers to the ability of a reverse transcriptase to exhibit enzyme activity at elevated temperatures.

As used herein, “thermostability” or “thermostable” refers to the ability of a reverse transcriptase to withstand exposure to elevated temperatures, but not necessarily show activity at such elevated temperatures. In some embodiments, thermostable reverse transcriptase or polymerase refers to any enzyme that catalyzes polynucleotide synthesis by addition of nucleotide units to a nucleotide chain using DNA or RNA as a template and has an optimal activity at a temperature above 53° C.

As sued herein, the terms “unique molecular identifier”, “unique molecular identifying sequence”, “UMI” and “UMI sequence” are used synonymously. Individual barcoded molecules may comprise a common barcode sequence such as a partition specific sequence or a spatial array where every capture probe has a unique barcode sequence.

By “binding sequence” is intended a nucleic acid sequence capable of binding to an analyte.

As used herein, the term “Variant” means a protein which is derived from a precursor protein (such as a wt MMLV protein, set forth in SEQ ID NO:15) by addition of one or more amino acids to either or both the C- and N-terminal end or at one or more sites in the amino acid sequence, substitution of one or more amino acids at one or more different amino acid sites in the amino acid sequence, or deletion of one or more amino acids at either or both ends of the protein or at one or more sites in the amino acid sequence. SEQ ID NO: 1 is a variant of MMLV and is generally used as a control enzyme unless noted otherwise. The preparation of an enzyme variant is preferably achieved by modifying a DNA sequence which encodes for the wild-type protein, transformation of that DNA sequence into a suitable host, and expression of the modified DNA sequence to form the derivative enzyme. It is recognized that the preparation of an enzyme variant may be achieved by modifying a DNA sequence which encodes for a variant of a wild-type protein, transformation of that DNA sequence into a suitable host, and expression of the modified DNA sequence to form the derivative enzyme. A variant reverse transcriptase of the invention includes altered amino acid sequences in comparison with a precursor enzyme amino acid sequence wherein the variant reverse transcriptase retains the characteristic enzymatic nature of the precursor enzyme but which may have altered properties in some specific aspect. For example, an engineered reverse transcriptase variant may have an altered pH optimum or increased temperature stability but may retain its characteristic transcriptase activity.

A “variant” may have at least about 45%, at least about 50%, at least about 55%, at least about 60%, at least about 65%, at least about 70%, at least about 75%, at least about 80%, at least about 85%, at least about 88%, at least about 90%, at least about 91%, at least about 92%, at least about 93%, at least about 94%, at least about 95%, at least about 96%, at least about 97%, at least about 98%, at least about 99%, or at least about 99.5% sequence identity to an amino acid sequence when optimally aligned for comparison. A variant residue position is described in relation to the wild-type amino acid sequence set forth in SEQ ID NO:15; otherwise said the amino acid position is indexed to SEQ ID NO:15.

A polypeptide having a certain percent (e.g., at least 90%, at least 91%, at least 92%, at least 93%, at least 94%, at least 95%, at least 96%, at least 97%, at least 98%, or at least 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY, Ausubel et al., eds., 1987, Supplement 30, section 7.7.18. Representative programs include the Vector NTI Advance™ 9.0 (Invitrogen Corp. Carlsbad, CA), GCG Pileup, FASTA (Pearson et al. (1988) Proc. Natl Acad. ScL USA 85:2444-2448), and BLAST (BLAST Manual, Altschul et al., Nat'l Cent. Biotechnol. Inf., Nat'l Lib. Med. (NCIB NLM NIH), Bethesda, Md., and Altschul et al., (1997) Nucleic Acids Res. 25:3389-3402) programs. Another typical alignment program is ALIGN Plus (Scientific and Educational Software, PA), generally using default parameters. Other sequence software programs that find use are the TFASTA Data Searching Program available in the Sequence Software Package Version 6.0 (Genetics Computer Group, University of Wisconsin, Madison, WI and CLC Main Workbench (Qiagen) Version 20.0.

| As used herein, the term “Sequencing,” generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

|0242| As used herein, the term “Wild-type” or “Wt” refers to a gene or gene product that has the characteristics of that gene or gene product when isolated from a naturally occurring source. The amino acid sequence set forth in SEQ ID NO:15 is a wt Murine Moloney Leukemia Virus (MMLV) sequence (Genbank NP_955591.1 p80 RT).

t will be understood that the reference to the below examples is for illustration purposes only and do not limit the scope of the claims. Each aspect, embodiment, or feature of the invention may be combined with any other aspect, embodiment, or feature the invention unless clearly indicated to the contrary. Unless defined otherwise, all technical and scientific terms used herein have the meaning commonly understood by a person skilled in the art to which this invention belongs.

### EXAMPLES

### Example 1. Capillary Electrophoresis Analysis of RT Mutant Enzymes

### Reverse Transcription and Sequencing Reactions.

The reaction volume was 50 μl; reactions contained 5′-end labeled GAPDH Primer, GEM-U reagent (Chromium 5′ Single Cell Assay, 10X Genomics), RNA template (GAPDH template), template switching oligo 1 (TSO1), and the indicated engineered reverse transcriptase. Stock concentrations and final concentrations in the reactions are shown in Table 1. The reactions included stoichiometrically equal amounts of enzyme and template for single turnover conditions. Reactants were incubated at 53° ° C. for 45 minutes, then diluted 1:20 in HiDi™ formamide (ThermoFisher). The formamide mixture was heated to 95° C. for 5 mins, then chilled on ice for 2 mins. Samples were loaded on a SeqStudio™ capillary electrophoresis genetic analyzer (ThermoFisher), a DS-33 Matrix Standard Dye Set G5 (ThermoFisher) was selected and long fragment analysis was performed using the GS1200LIZ size standard (GeneScan™ 1200 LIZ™, ThermoFisher). The GEM-U reagent approximates the formulation of the actual reagent mixture in a Chromium 5′ Single Cell GEM assay when the contents of the Z₁ and Z₂ channels are mixed.

TABLE 1
Capillary Electrophoresis Assay Reactants and
Template, Primer and TSO sequences (SEQ ID NOS:
18-20, respectively in order of appearance.)

| | | |
|---|---|---|
| Reagent | Stock | Final |

| | | | | |
|---|---|---|---|---|
| GEM-U Reagent | 4.00 | x | 1.00 | x |
| GAPDH mRNA | 5.6 | uM | 50 | nM |
| GAPDH Primer | 10 | uM | 25 | nM |
| TSO1.Oligo | 93.00 | uM | 12.00 | uM |
| Enzyme | Variable | uM | 0.8 | uM |

| | | |
|---|---|---|
| Water | — | — |

### Experimental Results.

FIGS. 6A-B provide exemplary results demonstrating the transcription (FIG. 6A) and template switching (FIG. 6B) efficiencies of eight different engineered MMLV RT variants as compared to a control MMLV RT comprising the amino acid of SEQ ID NO:1. All variants shown in the bar graphs demonstrated, to a lesser or greater extent, higher efficiencies then that of the control. The amino acid sequences of variant 1, variant 2, variant 3, variant 4, variant 5, variant 6 and variant 8 are set forth in SEQ ID NOs: 8, 9, 10, 11, 12, 13, and 14, respectively. The template switching efficiency of the variants having the amino acid sequences set forth in SEQ ID NOS: 8, 9, 10, 11, 12, 13, and 14 was greater than the template switching efficiency of the control SEQ ID NO:1. The amount of full-length product, an indicator of transcription efficiency, obtained from the variants having the amino acid sequences set forth in SEQ ID NOS: 8, 9, 10, 12, 13 and 14 was also greater than the control in SEQ ID NO:1.

FIG. 7 provides additional exemplary results demonstrating the transcription efficiencies (left bar of each set; darker grey) and template switching efficiencies (right bar of each set; light grey) for additional engineered MMLV RT variants compared to control SEQ ID NO:1, which serves as the control MMLV RT enzyme. The MMLV RT variants comprise the amino acid sequence of SEQ ID NOs: 2, 5, 4, 6, and 7. All MMLV RT variants, except for AB and AM, exhibited transcription efficiencies at or above about 40% shown by the control MMLV RT of SEQ ID NO: 1. Collectively, all MMLV RT variants, other than AM, exhibited higher transcription efficiencies than the control MMLV RT of SEQ ID NO: 1. MMLV variants AB, SEQ ID NO: 2, SEQ ID NO: 6 and SEQ ID NO: 7 exhibited template switching efficiencies that was higher than the 70% efficiency shown by the control MMLV RT of SEQ ID NO: 1. Variants SEQ ID NO: 2, SEQ ID NO: 6 and SEQ ID NO: 7 demonstrated increases in efficiency for both transcription efficiency and template switching efficiency over the control SEQ ID NO: 1.

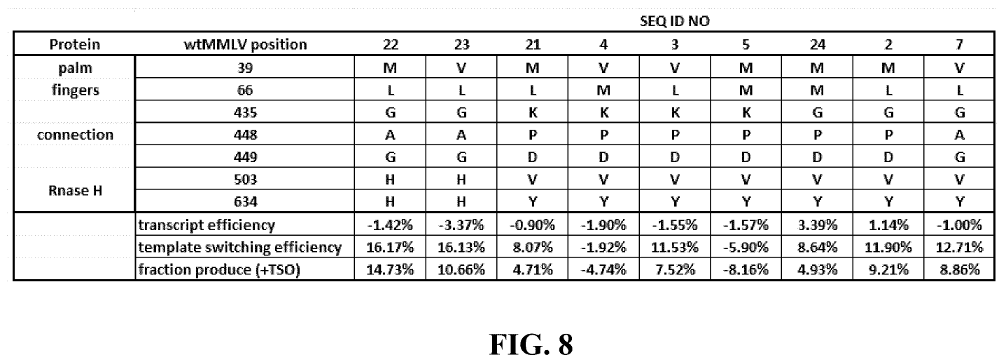

FIG. 8 shows additional MMLV variants (SEQ ID NOs: 2, 3, 4, 5, 7, 21, 22, 23, and 24) demonstrating similar levels of full-length product formation indicative of transcription efficiency. However SEQ ID NO:24 and SEQ ID NO:2 showed increase transcription efficiency over the control SEQ ID NO:1. It was noted that template switching efficiency and target product formation were improved in variants comprising a L435G or M66L mutation in SEQ ID NO: 15 (wt MMLV position). The improvement increased slightly when the variants were combination. Mutation M39V appeared to improve template switching (variant having the amino acid sequence set forth in SEQ ID NO:4 vs SEQ ID NO:5) but does little in combination with M66L. See results obtained from variants having the amino acid sequence set forth in SEQ ID NO:21 vs SEQ ID NO: 3, SEQ ID NO: 2 vs SEQ ID NO: 7, and SEQ ID NO: 22 vs SEQ ID NO: 23. Variants with one or more of the mutations P448A, D449G, H503V, and H634Y appear neutral in this context.

### Example 2. Single Cell 3′ and 5′ cDNA Yields

Various engineered reverse transcriptases were evaluated in single cell experiments with peripheral blood monocytes (PBMCs) at a cell load of 1,000, using either the 3′ and 5′ configurations (Chromium 3′ Single Cell Assay or Chromium 5′ Single Cell Assay, 10X Genomics). Emulsion droplets contained gel beads with either barcoded poly-dT primer sequences (3′ configuration) or barcoded with template switch oligo sequences (5′ configuration) that also include a UMI and Illumina Read 1 sequence. When cells were lysed within the droplet, the poly-dT primer hybridized to the poly-A tail of the cellular mRNA, which is extended by the reverse transcriptase. Once the end of the template is reached, the reverse transcriptase exhibits terminal transferase activity to add an overhang of three non-templated deoxycytidines (CCC) to the 3′ end of the synthesized cDNA. The CCC overhang hybridizes to the 3 riboguanosines (rGrGrG) present on the 3′ end of the template switch oligo, allowing the reverse transcriptase to “switch” templates and continue synthesis to the 5′ end of the template switch oligo. Depending on which configuration of gel bead is used (3′ or 5′) the barcode and UMI will allow either the 3′ or 5′-end of the mRNA molecule to be identified in the final sequencing library. Following reverse transcription at 53° C. for 45 mins, and a 5 min incubation at 85° C., droplets were broken and the cDNA was purified with Dynabeads®. The cDNA was amplified via PCR, purified with a 0.6×SPRI, and quantified with an Agilent BioAnalyzer using the DNA High Sensitivity Kit. The cDNA yield (ng) was determined.

FIG. 9 provides a summary of the cDNA yield from a series of experiments for the engineered reverse transcriptases having the amino acid sequence set forth in SEQ ID NOs: 1 (control), 22, 24, 2, 3 and 7. (n=2). Results from the 3′ configuration are shown as the left bar for each enzyme, and results from the 5′ configuration are shown as the right bar for each enzyme. Yields for variants with a M66L mutation (SEQ ID NO:2, 3, 7 and 22) and/or the M39V (SEQ ID NO: 3 or 7) mutation exceeded cDNA product yield over the control SEQ ID NO:1 in the 3′ experiments (SEQ ID NO:24 is not mutated at M39 or M66). These results were comparable to the results from tests of total product yield using a GAPDH mRNA template. Surprisingly, the cDNA product yields when using a single cell 5′ configuration differ from expectations based on the total product yield using a GAPDH mRNA template. For example, as shown in FIG. 9, all variants regardless of mutational status exceeded cDNA yield of control SEQ ID NO:1.

### Example 3. Single Cell 3′ Quality Metrics

Various variant reverse transcriptases were evaluated in single cell experiments using peripheral blood monocytes (PBMCs) using the 3′ and 5′ reaction conditions. Either 10 μL of amplified cDNA (3′ conditions) or 20 μL containing a maximum of 50 ng of amplified cDNA (5′ conditions) were fragmented and A-tailed, purified with double-sided SPRI (0.6×/0.8×) clean up, ligated to functional adaptors with an Illumina Read 2 sequence, purified with 0.8×SPRI, and further amplified with sample indexing primers that include the P5 and P7 priming sites and the i5 and i7 sample indexes. The amplification product was cleaned up with a double-side (0.6×/0.8×) SPRI, and the average size was determined with an Agilent BioAnalyzer using the DNA High Sensitivity Kit. The purified amplification product was quantified by qPCR and pooled for next generation sequencing on an Illumina NovaSeq™ targeting a sequencing depth of at least 50,000 reads per cell and using the following run parameters (Read 1: 28 cycles, i7 Index: 10 cycles, i5 Index: 10 cycles, Read 2: 90 cycles). Data was collected, demultiplexed, and processed. Standard quality metrics were obtained.

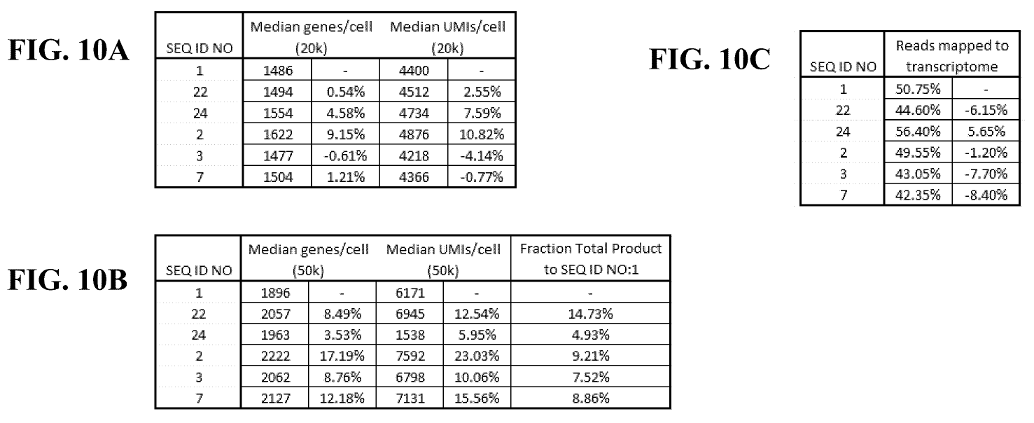

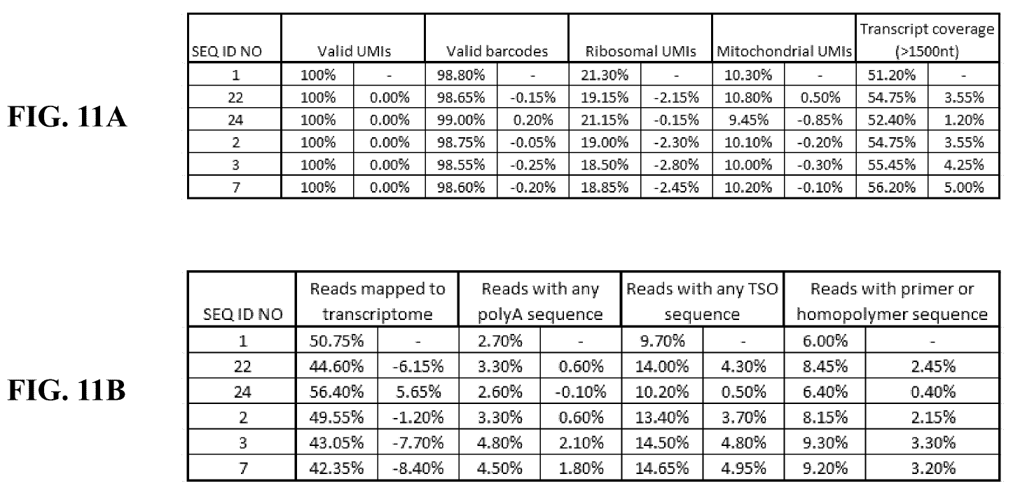

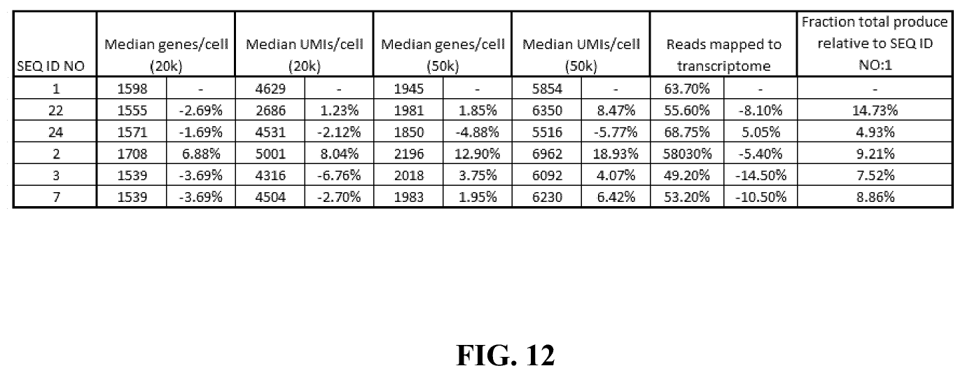

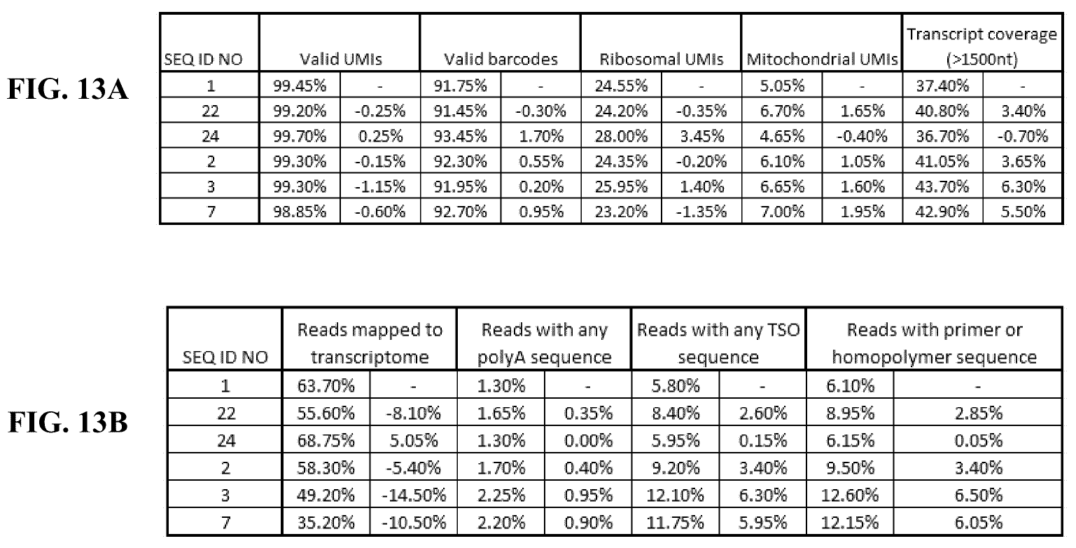

The single cell 5′ reactions use less enzyme and TSO oligo than the single cell 3′ reactions. The 5′ TSO oligo is also twice the length of the 3′ TSO oligo with varied sequence context due to the presence of the UMI and the barcode. The single cell 5′ reaction conditions are generally considered a more stringent test of performance than the 3′ single cell reaction conditions. Results from one such series of experiments (3′ reaction conditions) are summarized in FIG. 10 and FIG. 11. Results from one such series of experiments (5′ reaction conditions) are summarized in FIG. 12 and FIG. 13.

As shown in FIG. 10, all variants with a M66L mutation showed improved sensitivity at 50 k reads per cell but the extent of the improvement is contextual under 3′ reaction conditions. The trend correlated well with the capillary electrophoresis data with the variant engineered reverse transcriptase of SEQ ID NO:24 underperforming relative to the other variants. Surprisingly, only the variant of SEQ ID NO:2 showed significant improvement at 20 k reads/cell. The variant reverse transcriptase of SEQ ID NO:2 lacks the M39V mutation that is present in SEQ ID NO:3 and SEQ ID NO:7. Surprisingly, the M39V mutation improved template switching efficiency in vitro. However, when combined with M66L, the M39V mutation alone appeared not to provide significant additional benefit. Further, the variant engineered reverse transcriptase of SEQ ID NO:2 lacks the P448A and D449G mutations present in SEQ ID NO:1, 22 and 7. Surprisingly, SEQ ID NO:22 and 7 have similar sensitivities. The P448A and D449G mutations appear to not alter sensitivity in this context. Surprisingly, engineered reverse transcriptases with the M66L alteration, P448A, D449G and/or M39V suffer loss in mapping reads to the transcriptome. The exception is the engineered reverse transcriptase SEQ ID NO:2.

FIG. 11 shows that most of the variants yielded metrics within parity for valid UMI's, valid barcodes, ribosomal UMI's, mitochondrial UMI's, transcript coverage, reads with any poly(A) sequence, reads with any switch oligo sequence and reads with primer or homopolymer sequence under 3′ reaction conditions. However, when the libraries produced by some of the variants with the M66L mutation in combination with either P448A, D449G and/or M39V were evaluated for reads mapped to the transcriptome, there was a decrease in reads mapped to the transcriptome. Surprisingly, the variant of SEQ ID NO: 2, which includes M66L exhibited improved template switching efficiency and maintained levels of reads mapped to the transcriptome similar to the control RT of SEQ ID NO: 1.

FIG. 12 shows that, under 5′ reaction conditions, engineered reverse transcriptase variants having the amino acid sequence set forth in SEQ ID NO: 2 showed a significant improvement in sensitivity. Engineered reverse transcriptases with the M66L, P448A, D449G and/or M39V substitution suffered losses of mapping sequence reads to the transcriptome.

FIG. 13 shows that, under 5′ reaction conditions, most of the variants yielded metrics within parity for valid UMI's, valid barcodes, ribosomal UMI's, mitochondrial UMI's, transcript coverage, reads with any poly(A) sequence, reads with any switch oligo sequence and reads with primer or homopolymer sequence. However, when the libraries produced by most variants with the M66L mutation in combination with either P448A, D449G and/or M39V were evaluated for reads mapped to the transcriptome, there was a decrease in reads mapped to the transcriptome. Surprisingly the variant having the amino acid sequence set forth in SEQ ID NO: 2, which has the M66L mutation exhibited improved template switching efficiency and the levels of reads mapped to the transcriptome is impacted less than when other engineered reverse transcriptases are used.

### Example 4. Single Cell Sensitivity and Mapping

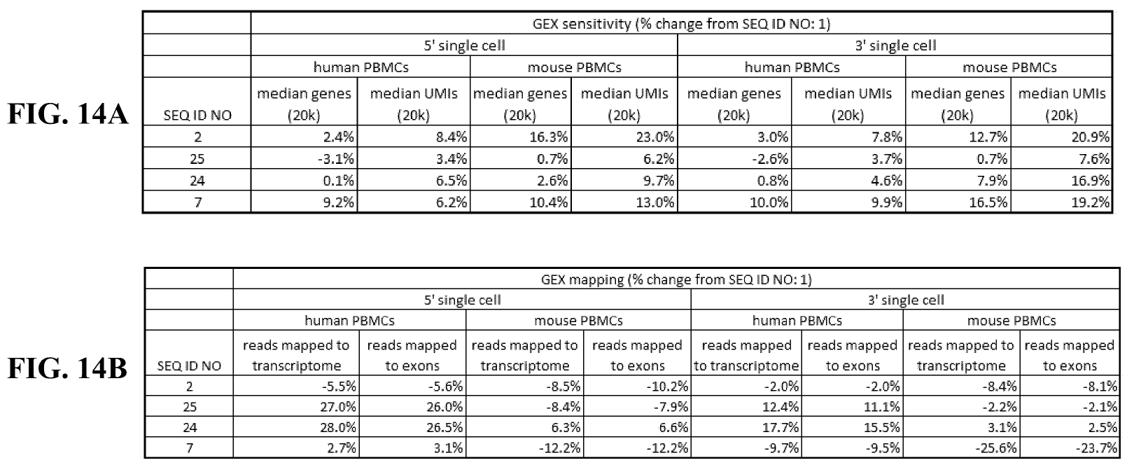

Various engineered reverse transcriptases (SEQ ID NOs: 2, 7, 24 and 25) were evaluated in single cell experiments with human peripheral blood monocytes (PBMCs) and mouse peripheral blood monocyte cells (C57B/L6) using 3′ and 5′ reaction conditions as described above herein. Sensitivity and mapping were evaluated. Results from engineered reverse transcriptases were compared to results obtained from a commercially available engineered MMLV. Results from one such series of experiments are summarized in FIG. 14.

In FIGS. 14A-B, the engineered reverse transcriptase variants were evaluated with human and mouse peripheral blood monocytes in 5′ and 3′ chemistries. The percent change is as compared to a commercially available MMLV reverse transcriptase as the control RT. The change in median genes and median UMI's queried at 20 k reads per cell (FIG. 14A) and the change in reads mapped to the transcriptome and reads mapped to exons (FIG. 14A) are shown. The amino acid sequences of the engineered reverse transcriptases are set forth in SEQ ID NO:2, SEQ ID NO:7, SEQ ID NO:24 and SEQ ID NO:25. As shown in FIGS. 14A-B, improvements in both the 5′ and 3′ chemistries were more pronounced in the mouse PBMC's than in the human PBMCs. Note the improvement in gene expression (GEX) sensitivity by the engineered reverse transcriptase having the amino acid sequence set forth in SEQ ID NO:2. It is also noted that the reads mapped to the transcriptome or the exon obtained with SEQ ID NO:2 decreased as compared to the control.

Further, t-Distributed Stochastic Neighbor Embedding (t-SNE) and scatter plots were used to evaluate the homogeneity of cell populations evaluated with engineered reverse transcriptase variants having the amino acid sequence set forth in SEQ ID NO:2 and SEQ ID NO:7 compared to SEQ ID NO:1 (control). Results from a t-SNE analysis and scatter plots are shown in FIGS. 15A-C.

The engineered reverse transcriptase having the amino acid sequence set forth in SEQ ID NO:2 exhibited tight correlation in both human and mouse samples as seen in the scatter plots for each variant (FIGS. 15A-B). The correlation exhibited by variant SEQ ID NO:2 was potentially better than that seen with SEQ ID NO:7, at least in human PBMC samples. (FIG. 15A). The engineered reverse transcriptase having the amino acid sequence set forth in SEQ ID NO:7 exhibited a tighter correlation in mouse cells than in human cells in 5′ and 3′ chemistries (3′ data not shown). As shown in FIG. 15C, an overlaid t-SNE plot by enzyme showed that the engineered reverse transcriptase having the amino acid sequence set forth in SEQ ID NO: 2 and SEQ ID NO:1 (control) show homogeneity in cell populations compared to the engineered reverse transcriptase having the amino acid sequence set forth in SEQ ID NO:7.

### Example 5. Immunoprofiling and TCR Improvements

Immune profiling is an extension of the 5′ chemistry to profile genes specifically for T-cell and/or B-cell receptors in the mRNA pool. Methods of immune profiling are known in the art and generally include additional rounds of PCR on the cDNA with a pool of sequence specific primers to allow for targeted enrichment of T-cell and/or B-cell receptor genes. Immune profiling assays may also detect UMIs for B-cell receptor genes, namely IGH, IGK, and IGL (Immunoglobulin heavy chain (IGH), kappa (IGK), and light (IGL) chain). Immune profiling data is informative for immunology research and is an extension of standard gene expression evaluation. Methods of immune profiling include, but are not limited to, Chromium Next Gen Single Cell™ kits (10X Genomics, Pleasanton CA).

To determine the efficiency of the novel MMLV RT variants (Table 2) disclosed herein in a single-cell V(D)J analysis, amplified cDNA (2 μl) from the 5′ configuration of reverse transcription reactions were subjected to two additional rounds of PCR enrichment with TCR immune profiling, which included a double-sided (0.5×/0.8×) SPRI clean-up between the first and second round of thermal cycling reactions. The amplified products were then cleaned-up with a subsequent double-sided (0.5×/0.8×) SPRI, fragmented and A-tailed, ligated to functional adaptors with an Illumina Read 2 sequence, cleaned up with a 0.8×SPRI, and then further amplified with sample indexing primers that include the P5 and P7 priming sites and the i5 and i7 sample indexes. The amplification product was cleaned up with a 0.8×SPRI, and average size was determined with an Agilent BioAnalyzer using the DNA High Sensitivity Kit. The material was then quantified by qPCR and pooled for next generation sequencing on an Illumina NovaSeq targeting a sequencing depth of at least 5,000 reads per cell and using the following run parameters (Read 1: 28 cycles, i7 Index: 10 cycles, 15 Index: 10 cycles, Read 2: 90 cycles). Data was collected, demultiplexed, and single-cell V(D)J analysis was performed.

Results obtained from engineered reverse transcriptases were compared to results obtained from the control SEQ ID NO:1. The percent change in median TRA UMI's and median TRB UMI's is shown in FIG. 16. FIG. 16 also shows the percent change in median IGH, IGK and IGL from mouse PBMC's. The median TRA UMIs and median TRB UMIs obtained with an engineered reverse transcriptase having the amino acid sequence set forth in SEQ ID NO: 2 were greater than those obtained with SEQ ID NO:1 in both human PBMCs and mouse PBMCs. Engineered reverse transcriptase variants previously shown to exhibit IG sensitivity exhibited a comparable or improved IG sensitivity (as compared to previous ATP results). In mouse PBMC's, the median IGH UMIs, median IGK UMIs and median IGL UMIs obtained with enzymes having the amino acid sequence set forth in SEQ ID NO:2, SEQ ID NO:25 or SEQ ID NO:24 were greater than those obtained with SEQ ID NO: 1 (right chart). The results obtained with an engineered reverse transcriptase having the amino acid sequence set forth in SEQ ID NO:2 were substantially higher than those obtained with engineered reverse transcriptases having the amino acid sequence set forth in SEQ ID NO:25 or SEQ ID NO:24. The improvement shown with mouse PBMCs was similar to the results observed with gene expression GEX (FIG. 14).

### INCORPORATION BY REFERENCE

All publications, patents, and patent applications mentioned in this specification are herein incorporated by reference 100% in their entireties to the same extent as if each individual publication, patent, or patent application was specifically and individually indicated to be incorporated by reference.

1.-32. (canceled)

33. An engineered Moloney Murine Leukemia Virus (MMLV) reverse transcriptase comprising relative to SEQ ID NO: 15:
(a) a M66L mutation and an L435G mutation; and
(b) one or more additional mutations selected from the group consisting of M39V, H503V, H634Y, P448A, and D449G.

34. The engineered MMLV reverse transcriptase of claim 33, wherein the engineered MMLV reverse transcriptase exhibits enhanced template switching activity as compared to SEQ ID NO: 1.

35. The engineered MMLV reverse transcriptase of claim 33, wherein the one or more additional mutations comprise P448A and D449G.

36. The engineered MMLV reverse transcriptase of claim 35, wherein the engineered MMLV reverse transcriptase comprises an amino acid sequence that is at least about 95%, at least about 97%, at least about 98%, or at least about 99% identical to SEQ ID NO: 7, SEQ ID NO: 22, or SEQ ID NO: 23.

37. The engineered MMLV reverse transcriptase of claim 35, wherein the engineered MMLV reverse transcriptase comprises an amino acid sequence that is at least about 95%, at least about 97%, at least about 98%, or at least about 99% identical to SEQ ID NO: 22.

38. The engineered MMLV reverse transcriptase of claim 35, wherein the engineered MMLV reverse transcriptase comprises an amino acid sequence as set forth in SEQ ID NO: 22.

39. The MMLV reverse transcriptase of claim 33, wherein the one or more additional mutations comprise M39V.

40. The engineered MMLV reverse transcriptase of claim 39, wherein the engineered MMLV reverse transcriptase comprises an amino acid sequence that is at least about 95%, at least about 97%, at least about 98%, or at least about 99% identical to SEQ ID NO: 7.

41. The engineered MMLV reverse transcriptase of claim 39, wherein the engineered MMLV reverse transcriptase comprises an amino acid sequence set forth in SEQ ID NO: 7.

42. The engineered MMLV reverse transcriptase of claim 39, wherein the engineered MMLV reverse transcriptase comprises an amino acid sequence that is at least about 95%, at least about 97%, at least about 98%, or at least about 99% identical to SEQ ID NO: 23.

43. The engineered MMLV reverse transcriptase of claim 39, wherein the engineered MMLV reverse transcriptase comprises an amino acid sequence as set forth in SEQ ID NO: 23.

44. The engineered MMLV reverse transcriptase of claim 33, wherein the one or more additional mutations comprise an H503V mutation and an H634Y mutation.

45. The engineered MMLV reverse transcriptase of claim 44, wherein the engineered MMLV reverse transcriptase comprises an amino acid sequence that is at least about 95%, at least about 97%, at least about 98%, or at least about 99% identical to SEQ ID NO: 2.

46. The engineered MMLV reverse transcriptase of claim 44, wherein the engineered MMLV reverse transcriptase comprises an amino acid sequence as set forth in SEQ ID NO: 2.

47. An isolated nucleic acid molecule encoding the engineered MMLV reverse transcriptase of claim 33.

48. An expression vector comprising the isolated nucleic acid of claim 47.

49. A host cell transfected with the expression vector of claim 48.

50. A method of using the engineered MMLV reverse transcriptase of claim 33, the method comprising contacting the engineered MMLV reverse transcriptase with a nucleic acid template under suitable conditions to produce a polymerized nucleic acid product, wherein the nucleic acid template is an RNA, a DNA, or a nucleic acid comprising an unnatural nucleotide.

51. A nucleic acid extension method comprising:
(a) contacting a target nucleic acid molecule with an engineered reverse transcriptase and a plurality of nucleic acid barcoded molecules comprising a barcode sequence, and
(b) incubating the target nucleic acid, the engineered reverse transcriptase and barcoded molecules under conditions in which the barcoded molecules are extended by the engineered reverse transcriptase,
wherein the engineered reverse transcriptase comprises the amino acid sequence of an engineered MMLV transcriptase of claim 33.

52. The method of claim 51, wherein the method is performed in a reaction volume of 1 nl or less.


SEQUENCE LISTING (45 sequences)

SEQ ID NO: 1 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 2 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 3 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 4 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 5 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 6 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 7 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 8 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 9 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNHMAHQAARRAAITETPDTSTLP

SEQ ID NO: 10 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 11 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAERWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTDSRYAFATAHIHGEIYRRRGWLTSGGKEIKNKDEILALLKALFLPKRLSIIHCPGHQKGHSVEARGNRMAHQAARRAAITETPDTSTLP

SEQ ID NO: 12 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 13 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFEEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTDGSSLLQEGQRKAGAAVTTETGVIWAKALPAGTSAQRAELIALTQALKMAEGKKLNVYTNSRYAFATAHIQGEIYRRRGFLTSKGKEIKNKDEILALLKALFLPKRLSIIHCPGHQKGHSAEARGNRMADQAARKAAITETPDTSTLP

SEQ ID NO: 14 - protein, 671 aa, synthetic construct
  REGION 1..671: note = Description of Artificial Sequence: Synthetic polypeptide
TLNIEDEHRLHETSKEPDVSLGSTWLSDFPQAWAETGGVGLAVRQALLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMSQPLVIKAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTDETEVIWAKALPAGTSAQRAELIALTQALKMAEGKKLNVYTNSRYAFATAHIHGEIYRRRGWLTSGGKEIKNKDEILALLKALFLSKRLSIIHCPGHQKGHSVEARGNRMAHQAARRAAITETPDTSTLP

SEQ ID NO: 15 - protein, 671 aa, Murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 16 - protein, 5 aa, synthetic construct
  REGION 1..5: note = Description of Artificial Sequence: Synthetic 5xHis tag
  REGION 1..5: note = See specification as filed for detailed description of substitutions and preferred embodiments
HHHHH

SEQ ID NO: 17 - DNA, 10 bp, synthetic construct
  misc_feature 1..10: note = Description of Artificial Sequence: Synthetic oligonucleotide
  misc_feature 1..10: note = See specification as filed for detailed description of substitutions and preferred embodiments
  source 1..10: mol_type = other DNA
tttttttttt

SEQ ID NO: 18 - RNA, 1315 nt, synthetic construct
  misc_feature 1..1315: note = Description of Artificial Sequence: Synthetic polynucleotide
  source 1..1315: mol_type = other RNA
agagggaaagcagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 19 - DNA, 29 bp, synthetic construct
  misc_feature 1..29: note = Description of Artificial Sequence: Synthetic primer
  source 1..29: mol_type = other DNA
tggttgagcacagggtactttattgatgg

SEQ ID NO: 20 - DNA, 30 bp, synthetic construct
  misc_feature 1..30: note = Description of Artificial Sequence: Synthetic oligonucleotide
  source 1..30: mol_type = other DNA
aagcagtggtatcaacgcagagtacatggg

SEQ ID NO: 21 - skipped

SEQ ID NO: 22 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 23 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 24 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 25 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPAGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 26 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 27 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPADRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 28 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPGRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 29 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAVGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 30 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIYCPGHQKGHSAEARGNRMADQAARKAAITETPDTSTLL

SEQ ID NO: 31 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 32 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 33 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 34 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGGQKGHSAEARGNRMADQAARKAAITETPDTSTLL

SEQ ID NO: 35 - protein, 649 aa, synthetic construct
  REGION 1..649: note = Description of Artificial Sequence: Synthetic polypeptide
MTWLSDFPQAWAETGGVGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 36 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAYFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 37 - protein, 649 aa, synthetic construct
  REGION 1..649: note = Description of Artificial Sequence: Synthetic polypeptide
MTWLSDFPQAWAETGGMGLAVRQAPLIIPLKATSTPVSIKQYPLSQKARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLL

SEQ ID NO: 38 - protein, 6 aa, synthetic construct
  REGION 1..6: note = Description of Artificial Sequence: Synthetic 6xHis tag
HHHHHH

SEQ ID NO: 39 - protein, 11 aa, synthetic construct
  REGION 1..11: note = Description of Artificial Sequence: Synthetic peptide
SEEDEEKEEDG

SEQ ID NO: 40 - protein, 7 aa, synthetic construct
  REGION 1..7: note = Description of Artificial Sequence: Synthetic peptide
ENLYFQG

SEQ ID NO: 41 - protein, 5 aa, synthetic construct
  REGION 1..5: note = Description of Artificial Sequence: Synthetic peptide
DDDDK

SEQ ID NO: 42 - protein, 4 aa, synthetic construct
  REGION 1..4: note = Description of Artificial Sequence: Synthetic peptide
IEGR

SEQ ID NO: 43 - protein, 6 aa, synthetic construct
  REGION 1..6: note = Description of Artificial Sequence: Synthetic peptide
LVPRGS

SEQ ID NO: 44 - protein, 8 aa, synthetic construct
  REGION 1..8: note = Description of Artificial Sequence: Synthetic peptide
LEVLFQGP

SEQ ID NO: 45 - RNA, 11 nt, synthetic construct
  misc_feature 1..11: note = Description of Artificial Sequence: Synthetic oligonucleotide
  source 1..11: mol_type = other RNA
aaaaaaaaaaa